(19) United States
(12) Patent Application Publication
(10) Pub. No.: US 2024/0198316 A1
(43) Pub. Date: Jun. 20, 2024

(54) SYNTHETIC POLYMERIC POROUS MEDIUM WITH HIERARCHICAL MULTIPLE LAYER STRUCTURE, ITS DESIGN, SYNTHESIS, MODIFICATION, AND LIQUID CHROMATOGRAPHIC APPLICATIONS

(71) Applicant: Suzhou Sepax Technologies, Inc., Suzhou, Jiangsu (CN)

(72) Inventors: Huiming MAO, Suzhou, Jiangsu (CN); Feng XU, Suzhou, Jiangsu (CN); Ke YANG, Suzhou, Jiangsu (CN); Xinmei HU, Suzhou, Jiangsu (CN); Xueying HUANG, Suzhou, Jiangsu (CN)

(21) Appl. No.: 18/556,895

(22) PCT Filed: May 30, 2022
(86) PCT No.: PCT/CN2022/095945

§ 371 Date: Oct. 24, 2023

(30) Foreign Application Priority Data
May 31, 2021 (WO) PCT/CN2021/097462
Jun. 24, 2021 (CN) 202110704351.0

(51) Int. Cl. B01J 20/288; B01D 15/20; B01D 15/32; B01D 15/34; B01D 15/36; B01D 15/38; B01J 20/26; B01J 20/28; B01J 20/289; B01J 20/30; B01J 20/32

(52) U.S. Cl. CPC B01J 20/288; B01D 15/206; B01D 15/327; B01D 15/34; B01D 15/362; B01D 15/363; B01D 15/3809; B01D 15/3847; B01J 20/267; B01J 20/28004; B01J 20/28016; B01J 20/28059; B01J 20/28064; B01J 20/28066; B01J 20/28071; B01J 20/28073; B01J 20/28076; B01J 20/28083; B01J 20/28085; B01J 20/289; B01J 20/3064; B01J 20/3085; B01J 20/321; B01J 20/3221; B01J 20/3244; B01J 20/3293; B01J 2220/54

(57) ABSTRACT

A synthetic polymeric porous medium with a core-shell(s) hierarchical layer structure and has an essentially homogeneous porous structure from inside to outside of the medium, whose core and shell(s) are covalently modified with distinct chemical functional groups or same functional group with different density. Here the methodologies for resin syntheses and core-shell(s) modifications and liquid chromatographic applications of the newly developed resins in the field of analysis and purification of Tween surfactants, virus-like particles (VLP)/vaccines/viral vectors/viruses, antibody, and mRNA are disclosed.

### CROSS-REFERENCE TO RELATED APPLICATIONS

This application is a Section 371 of International Application No. PCT/CN2022/095945 filed May 30, 2022, which was published in the English language Dec. 8, 2022, under International Publication No. WO 2022/253175 A1, which claims priority to International Application No. PCT/CN2021/097462 filed May 31, 2021, and Chinese Patent Application No. 202110704351.0 filed Jun. 24, 2021, the disclosures of which are incorporated herein by reference in their entireties.

### TECHNICAL FIELD

The present invention relates to the field of liquid chromatography, and in particular to a synthetic polymeric porous medium with hierarchical multiple layer structure and essentially homogeneous porous structure from inside to outside of the medium, its design, synthesis, modification, and liquid chromatographic applications.

### BACKGROUND OF THE INVENTION

Liquid Chromatography (LC) is an important tool for the separation of substance(s) from a mixture. As the key component in modern LC, LC media, solid porous supports, can be categorized into two large categories: inorganic LC media (silica and related inorganic oxides) and organic LC media. While organic LC media can be further divided into nature polymers such as agarose, cellulose, dextran, chitosan, and their derivatives, and synthetic polymers.

LC is playing more and more important roles in chemistry, biochemistry, pharmaceutical industries, and other cutting-edge fields of science, and drawing more and more attention from both academics and industries. In general, LC involves and plays very important role in all the pharmaceutical processes, including discovery, development, manufacturing process, and quality control. For example, it is widely used for identifying and analyzing samples for the presence of chemicals or trace elements, preparing huge quantities of extremely pure materials, separating chiral compounds, detecting the purity of mixture and the unknown compounds, and isolating and purifying drugs in large scale.

Biological products (biologics), emerging as new and important therapeutic agents, cover a wide range of products, such as various recombinant therapeutic proteins, vaccines, blood, and blood components, allergenics, cells, gene therapy, and tissues. Biologics usually composed of sugars, proteins, or nucleic acids or their complex combinations can be isolated from a variety of natural sources such as human, animal, or microorganism, or produced by biotechnology methods and other cutting-edge technologies. They often are at the forefront of biomedical research, and may be used to treat a variety of serious diseases for which no other treatments are available.

However, there are many challenges and unsettled problems in biologic drugs discovery, development, and manufacturing due to their heterogenic physicochemical characteristics and complexity of separation mixtures. For example, 1) Their molecular weight (MW), charges, and post-transitional modification are different, so advanced LC characterization and fast QC analysis technologies are essential. 2) Impurities, which may be product-related or process-related, must be removed and characterized, so multiple large-scale and high purity purification processes are usually needed. 3) Due to their poor stability and low concentration, downstream purification processes must be optimized, which aim at increasing production efficiency and decreasing biologics drug cost. For example, cutting down or simplifying LC process steps, increasing purification speed, reducing buffer consumption and waste creation all can help increase manufacturing throughput and reduce manufacturing cost.

As known, separation and purification of biologics mainly rely on chromatographic technology. These unmet and challenging needs mentioned above provide opportunities for new LC medium development. This is especially the case as the number of therapeutics biologics in R&D and commercialization pipeline continues to increase, the complexity of their structures increases, and the requirements for analysis and separation increase accordingly.

Although new LC media emerges from time to time, they in general do not keep up with the high demands from biopharmaceutical industries for several reasons: 1) Biopharmaceutical industries need LC platform product, which enable systematic and platform solution to new biologics in terms of DSP without requiring a new and usually lengthy process development for each new biologic product. 2) Lack of scalability in terms of LC media of choice in different commercialization phase of a biologic drug. This is problematic from analytical characterization to production, and from small scale to full scale production, while many LC media are only commercially available in a certain range of bead size and pore size, and have limited choices of resin chemistry. There is no guarantee a commercial LC medium is available for full scale production even if initial analytical results or small-scale purification results are good. 3) Lack of versatile resin chemistry or separation mode to meet individual separation/purification challenges. Industries not only need some LC media with conventional performance to keep their cost low, but also need customized LC media (or LC media that could be readily customized) to increase manufacturing efficiency.

### SUMMARY OF THE INVENTION

For overcoming said problems and satisfying liquid chromatography (LC) separation requirements in related fields, as just described above, the present invention provides design, synthesis, modification, and applications of a novel polymeric porous chromatography medium with a core-shell(s) hierarchical layer structure and essentially homogeneous porous structure from inside to outside of the medium. The innovative chromatographic medium of the present invention, with narrow size distribution and desired porous structure, combining a size exclusion separation and various binding chemistry, is a platform tool to solve many challenging tasks in analytical and industrial fields, such as reducing purification process steps, increasing sample loading in biologics downstream processes, and meeting ever increasing challenges and demands from the analytical needs of biologics.

In the first aspect of present invention, a synthetic polymeric porous chromatography medium is provided, wherein the chromatography medium has a hierarchical multiple layer structure and essentially homogeneous porous structure from inside to outside of the medium, wherein the hierarchical multiple layer structure is made of synthetic polymer and has pores for size exclusion separation; and at least one inner layer and at least one outer layer in the hierarchical multiple layer structure have different binding functional groups (or LC functional groups), or have same binding functional group with different density so that the chromatographic property of said at least one inner layer is different from that of said at least one outer layer.

In another preferred embodiment, the chromatography medium has core-shell(s) structure.

In another preferred embodiment, the hierarchical multiple layer structure has 2, 3, or 4 layers;
preferably, the hierarchical multiple layer structure has 2 layers, and the at least one inner layer is the core of chromatography medium, and the at least one outer layer is shell of the chromatography medium.

In another preferred embodiment, the binding function group is selected from the group consisting of hydrophobic groups, hydrophilic groups, ionic or ionizable groups, affinity groups, mixed-mode groups and combinations thereof;
preferably, the hydrophobic group is selected from the group consisting of linear or branched alkyl chain (C1-C18), oligo(ethylene oxide), phenyl, benzyl groups, and their derivatives, which are linked to the polymeric matrix through oxygen atom (O), nitrogen atom (N), sulfur atom (S), ether, ester, or amide groups;
preferably, the hydrophilic group is hydroxy, or a group that is converted through a chemical modification with 2-hydroxyethanethiol, 3-sulfanylpropane-1,2-diol, Dextran, any linear or branched multifunctional epoxide;
preferably, the ionic or ionizable group is selected from the cationic group consisting of primary amine, secondary amine, tertiary amine, or combinations thereof;
preferably, the primary amine is linear or branched alkylamine C1-C18; more preferably, the primary amine is selected from the group consisting of ethylamine, butylamine, hexylamine, octylamine, or combinations thereof;
preferably, the secondary amine is selected from the group consisting of dimethylamine, diethylamine, or combinations thereof;
preferably, the tertiary amine is selected from the group consisting of trimethylamine, N, N-dimethylbutylamine, or combinations thereof;
preferably, the ionic or ionizable group is selected from the anionic group consisting of sulfonate, phosphate, carboxylate, and their derivatives;
preferably, the affinity group is selected from the group consisting of Protein A, Protein L, Protein G, 3-aminophenylboronic acid, sense/antisense oligonucleotide, iminodiacetic acid (IDA), tri(carboxymethyl)ethylene diamine (TED), nitrilotriacetic acid (NTA), and other metal chelating ligands;
preferably, the mixed-mode group is selected from the group consisting of secondary amine and tertiary amines containing at least one linear alky group (C2-C10), N, N-Dimethylbutylamine, N-Benzyl-N-methylethanolamine, N, N-Dimethylbenzylamine, and 2-benzamido-4-mercaptobutanoic acid.

In another preferred embodiment, the chromatography medium has one or more of the following features:
(a) specific pore volume in a range of 0.05-3.0 mL/g, preferably from 0.2 to 2.5 mL/g, most preferably from 0.4 to 2.0 mL/g;
(b) specific surface area in a range of 40-1200 m²/g, preferably from 60 to 1000 m²/g, most preferably from 80 to 800 m²/g;
(c) average pore size in a range of 30-5000 Å, more preferably from 50 Å to 3000 Å, most preferably from 100 Å to 2000 Å; and preferably, the average pore size is essentially homogeneous from inside to outside of the porous mother medium;
(d) volume average particle diameter (D₅₀) in a range of 1-1000 μm, more preferably from 1 μm to 500 μm, most preferably from 2 μm to 200 μm;
(e) particle size distribution (D₉₀/D₁₀) in a range of 1.0-2.2, preferably 1.0-1.5, more preferably 1.0-1.2, most preferably 1.0-1.05.

In another preferred embodiment, the chromatography medium is made from a mother medium.

In another preferred embodiment, the mother medium is copolymerized from a monomer mixture which comprises:
(M1) at least a first monomer which is a crosslinking monomer;
(M2) at least a second monomer which comprises a monomer with a convertible functional group for hierarchical structure construction, and
(M3) an optional third monomer which has a special functional group for tuning chromatographic properties;
preferably, the first monomer and the second monomer are the same monomer;
preferably, the first monomer and the third monomer are the same monomer;
preferably, the second monomer and the third monomer are the same monomer;
preferably, the first monomer, the second monomer and the third monomer are same or different monomer.

In another preferred embodiment, the mother medium has one or more of the following features:
(a) specific pore volume in a range of 0.05-3.0 mL/g, preferably from 0.2 to 2.5 mL/g, most preferably from 0.4 to 2.0 mL/g;
(b) specific surface area in a range of 40-1200 m²/g, preferably from 60 to 1000 m²/g, most preferably from 80 to 800 m²/g;
(c) average pore size in a range of 30-5000 Å, more preferably from 50 Å to 3000 Å, most preferably from 100 Å to 2000 Å; and preferably, the average pore size is essentially homogeneous from inside to outside of the porous mother medium;
(d) volume average particle diameter (D₅₀) in a range of 1-1000 μm, more preferably from 1 μm to 500 μm, most preferably from 2 μm to 200 μm;
(e) particle size distribution (D₉₀/D₁₀) in a range of 1.0-2.2, preferably 1.0-1.5, more preferably 1.0-1.2, most preferably 1.0-1.05;
(f) alkene content of the mother medium in a range of 0.5-6.0 mmol/g, preferably 0.7-5.5 mmol/g, most preferably 0.9-5.2 mmol/g.

In another preferred embodiment, the shape and/or form of the chromatography medium is a substantially flat particulate or monolithic rod or disk, the most preferred shape of a particulate is spherical or pseudo-spherical.

In another preferred embodiment, the mother medium has one or more of the following features:
(F1) the first monomer or crosslinking monomer accounts for 1-99% wt of all monomers used in copolymerization process;
preferably, said crosslinking monomer is selected from the group consisting of (meth)acrylic, styrenic, and vinylic monomers;
more preferably, said crosslinking monomer is selected from the group consisting of divinylbenzene (DVB), ethylene glycol dimethacrylate, pentaerythritol dimethacrylate, pentaerythritol triacrylate, pentaerythritol tetraacrylate, sorbitol dimethacrylate, poly(ethylene glycol) diacrylate, poly(propylene glycol) diacrylate, trimethylolpropane triacrylate, bis2-(methacryloyloxy)ethyl phosphate, N,N′-methylenebisacrylamide, 3-(acryloyloxy)-2-hydroxypropyl methacrylate, glycerol 1,3-diglycerolate diacrylate, 1,5-hexadiene, allyl ether, diallyl diglycol carbonate, di(ethylene glycol) bis(allyl carbonate), ethylene glycol bis(allyl carbonate), triethylene glycol bis(allyl carbonate), tetraethylene glycol bis(allyl carbonate), glycerol tris (allyl carbonate), ethylene glycol bis(methallyl carbonate), diallyl phthalate, triallyl isocyanurate, diallyl isophthalate, diallyl terephthalate, diallyl itaconate, diallyl 2,6-naphthalene dicarboxylate, diallyl chlorendate, triallyl trimellitate, triallyl citrate, 2,4,6-triallyloxy-1,3,5-triazine, 1,3,5-triacryloylhexahydro-1,3,5-triazine, glyoxal bis(diallyl acetal), N,N-diallyldimethyl ammonium salts, ethylene glycol diallyl ether and combinations thereof;
(F2) the second monomer accounts for 1-99% wt of all monomers used in copolymerization process;
preferably, the second monomer is selected from the group consisting of (meth)acrylate, acrylamide, ethylene terephthalate, ethylene, propylene, styrene, vinyl acetate, vinyl acrylate, vinyl chloride, vinyl pyrrolidone, DVB, 1,3,5-trivinylbenzene, and combinations thereof;
preferably, the second monomer contains at least one inactive, less active, or protected functional group which can survive during polymerization process, and then further be used in hierarchical modification directly or indirectly;
preferably, said convertible functional group is selected from the group consisting of amino, sulfenyl, benzyl, phenyl, alkyl, alkynyl, hydroxyl, carboxyl, aldehyde, halo, thiol groups and combinations thereof;
preferably, said convertible functional group is alkenyl; preferably, the alkenyl has a carbon-carbon double bond; more preferably, the convertible functional group is selected from the group consisting of allyl and vinyl;
more preferably, said alkenyl is from (meth)acrylic, styrenic, and/or vinylic monomers; preferably, the second monomer is selected from the group consisting of allyl acrylate, allyl methacrylate, vinyl acrylate, diallyl maleate (DAM), DVB, 1,3,5-trivinylbenzene, and combinations thereof;
most preferably, the second monomer is allyl methacrylate and/or diallyl maleate;
(F3) the third monomer accounts for 1-99% wt of all monomers used in copolymerization process;
preferably, the third monomer is selected from the group consisting of (meth)acrylic, styrenic, and vinylic monomers;
more preferably, said third monomer is selected from the group consisting of glycidyl methacrylate, 2-hydroxyethyl methacrylate, 2-carboxyethyl acrylate, 2-hydroxyethyl acrylate, 2-hydroxyethyl methacrylate, methyl methacrylate, methacrylic acid, hydroxypropyl methacrylate, 2-(methacryloyloxy)ethyl acetoacetate, mono-2-(methacryloyloxy)ethyl maleate, benzyl acrylate, butyl acrylate, styrene, DVB, N-Vinylpyrrolidone and combinations thereof.

In another preferred embodiment, chromatography medium has one or more of the following features:
(T1) the chromatography medium with core-shell(s) structure has at least two layers, where the core refers to the most inner layer, while the shell(s) refer(s) to the outer layer(s) from the core; preferably, the core and each shell layer are spatially defined with distinct functional groups and relative spatial layout between each layer; more preferably, said hierarchical structures is core-shell (two-layer) structure with chemically distinct functional groups or same functional group with different density in each layer;
(T2) the chromatography medium with core-shell structure is composed of a hydrophilic shell and cationic ligand(s) activated core with or without a linker; preferably, said cationic ligand is selected from the group consisting of ammonium, sulfonium, phosphonium, primary amines, secondary amines, tertiary amines, and combinations thereof,
preferably, the primary amine is selected from the group consisting of ethylamine, butylamine, hexylamine, octylamine, and combinations thereof;
preferably, the secondary amine is selected from the group consisting of dimethylamine, diethylamine, and a combination thereof;
preferably, the tertiary amine is selected from the group consisting of trimethylamine, N,N-dimethylbutylamine, and a combination thereof,
(T3) the chromatography medium with core-shell structure is composed of a hydrophilic shell and anionic ligand(s) activated core with or without a linker; preferably, said anionic ligand can be any suitable sulfonate, phosphate, carboxylate, and their derivatives;
(T4) the chromatography medium with core-shell structure is composed of a hydrophilic shell and hydrophobic ligand(s) activated core with or without a linker; preferably, said hydrophobic ligand(s) can be any suitable hydrophobic group(s) linked to the backbone through oxygen atom (O), nitrogen atom (N), sulfur atom (S), ether, ester, or amide groups, such as linear or branched alkyl chain (C1-C18), oligo(ethylene oxide), phenyl, benzyl groups, and their derivatives;
(T5) the chromatography medium with core-shell structure is composed of a hydrophilic shell and affinity ligand(s) activated core with or without a linker; preferably, said affinity ligand(s) can be any ligand, or any suitable one with a strength of binding interaction to its binding partner; more preferably, the ligand is selected from the group consisting of Protein A, 3-aminophenylboronic acid, sense/antisense oligonucleotide, iminodiacetic acid (IDA), tri(carboxymethyl)ethylene diamine (TED), nitrilotriacetic acid (NTA), and combinations thereof;
(T6) the chromatography medium with core-shell structure is composed of a hydrophilic shell and mixed-mode ligand(s) activated core with or without a linker; preferably, said mixed-mode ligand, a stationary phase ligand composed of at least one hydrophobic moiety at peripheral position or branch position, and at least one ionic or ionizable groups at peripheral position or branch position, or embedded in the hydrophobic moiety, is selected from the group consisting of alkylamine, N, N-Dimethylbutylamine, N-Benzyl-N-methylethanolamine, N, N-Dimethylbenzylamine, and 2-benzamido-4-mercaptobutanoic acid;
preferably, the alkylamine is selected from the group consisting of ethylamine, butylamine, hexylamine, octylamine, and combinations thereof;
(T7) the chromatography medium has a cationic shell, which is modified with any suitable reagent leading to positively charged ligands, and a hydrophobic ligand(s) activated core, which can carry any hydrophobic ligands;
(T8) the chromatography medium has an anionic shell, which is modified with any suitable reagent leading to negatively charged ligands, and a hydrophobic ligand(s) activated core, which can carry any hydrophobic ligands;
(T9) the chromatography medium has an ionic or ionizable shell, which is modified with any suitable reagent leading to said ionic or ionizable ligands, and a hydrophilic core;
(T10) the chromatography medium is modified with the same ligand(s) in both the core layer and the shell layer, but with a different functional group(s) density; preferably, said ligand(s) can be any mentioned above;
(T11) the hydrophilicity in each shell of the chromatography medium can be tuned and enhanced through chemical modification with 2-hydroxyethanethiol, 3-sulfanylpropane-1,2-diol, Dextran, any linear or branched multifunctional epoxide, or any other agents with hydrophilic functional groups;
(T12) the chromatography medium can be physically converted/transformed into LC columns or other confined devices for molecular separations and purifications; preferably, the LC column or device is selected from the group consisting of analytical column, guard column, preparative column, semi-prep column, HPLC column, UPLC column, UHPLC column, FPLC column, flash column, gravity column, capillary column, spin column, disposable column, monolithic column, cartridge, plate and combinations thereof,
preferably, the LC column or device: a) is applied in batch mode or continuous mode such as counter current chromatography; b) has ID from 0.1 millimeter to 2 meters and a length from 1 millimeter to 2 meters; or 3) is used as single column or multi-column format in continuous or non-continuous (conventional) chromatography;
(T13) the chromatography medium with core-shell two-layer structure and designed pore size is used for analytical and preparative separations; preferably, the chromatography medium combines size exclusion separation and various binding chemistry, wherein larger molecules are excluded from the no-binding shell, and analyzed or collected as flow-through, while smaller molecules penetrate through the pore, and are temporarily trapped/bound into the functionalized core of the separation medium, which can be analyzed or collected in a bind-elute mode later; preferably, the separation sample comprising at least two substances with distinct molecular weight, wherein molecular weight ratio M1/M2≥2; preferably M1/M2≥5, most preferably M1/M2≥10, where M1 refers to the largest substance, and M2 refers to the smallest substance in the separation mixture; preferably, such a LC medium can be packed into a LC column for LC applications;
(T14) the chromatography medium combines anionic exchange adsorptive and size exclusion mechanisms, where a large substance, such as large protein, virus, large DNA, is analyzed or collected in flow-through mode, while a small substance is temporarily trapped/bound in the core, and then eluted for analysis or collection;
(T15) the chromatography medium combines cationic exchange adsorptive and size exclusion mechanisms, where a large substance, such as large protein, virus, large DNA, is analyzed or collected in flow-through mode, while a small substance is temporarily trapped/bound in the core, and then eluted for analysis or collection;
(T16) the chromatography medium combines hydrophobic adsorptive and size exclusion mechanisms, where a large substance, such as large protein, virus, large DNA, is analyzed or collected in flow-through mode, while a small substance is temporarily trapped/bound in the core, and then eluted for analysis or collection;
(T17) the chromatography medium combines affinity adsorptive and size exclusion mechanisms, where a large substance, such as large protein, virus, large DNA, is analyzed or collected in flow-through mode, while a small substance is temporarily trapped/bound in the core, and then eluted for analysis or collection;
(T18) the chromatography medium combines mixed-mode adsorptive and size exclusion mechanisms, where a large substance, such as large protein, virus, large DNA, is analyzed or collected in flow-through mode, while a small substance is temporarily trapped/bound in the core, and then eluted for analysis or collection;
(T19) the chromatography medium is applied to separate biomolecules from the surfactants used in stabilizing biotherapeutic formulation; preferably, the biomolecules can be therapeutic proteins with molecular weight range of 10 KD-3 MD; the surfactants can be polysorbates including Tween 20, 40, 60, and 80, polyethyleneoxide, poly(propylene oxide), sorbitan esters, ethoxylates, PEG, Poloxamer 188, Trion X-100, Miglyol, and maltosides including n-dodecyl-β-D-maltoside (DDM), n-octyl-β-D-maltoside (ODM);
(T20) the chromatography medium is applied to separate mixtures of large or super biomolecule assemblies natural or artificially-made such as eukaryotic and prokaryotic cells, VLPs, vaccines, viral vectors, viruses, viral vectors, or liposomes or LNPs (lipid nanoparticles) from small molecules or assemblies via the interactions at inner core different from that at outer layer;
preferably, said large or super biomolecule assemblies such as eukaryotic and prokaryotic cells, VLPs or vaccines, virus, viral vectors, or liposomes are in the in size >10 nm; said virus can be active or inactivated, enveloped or nonenveloped; such VLPs, vaccines, viral vectors, viruses, viral vectors, or liposomes or LNPs (lipid nanoparticles) can encapsulate genetic materials such as ssDNA, dsDNA, ssRNA, dsRNA; such liposome and lipid nanoparticle (LNP) can carry positive charge or negative charge or no charge, preferred entity carries positive charge;
preferably, said small molecule or assemblies includes but not limited to DNA fragment, RNA, plasmids, HCP, protein fragments, capsid proteins, endotoxins, detergents, benzonase, excessive components (unencapsulated components), with a size <10 nm.

In another preferred embodiment, chromatography medium has an affinity ligand.

In another preferred embodiment, chromatography medium is selected from the group consisting of:
(A1) a chromatography medium bearing an affinity ligand Protein A attached in its inner core, which is preferably applied to separate a mixture of a Fc containing protein;
(A2) a chromatography medium bearing an affinity ligand Protein L attached in the inner core, which is preferably applied to separate a mixture of Fab or kappa light chain containing proteins;
(A3) a chromatography medium bearing an affinity ligand Protein G attached in the inner core, which is preferably applied to separate a mixture of Fc and Fab containing proteins;
(A4) a chromatography medium bearing an affinity ligand oligonucleotide (e.g. dTs) which is preferably applied to mixture of oligonucleotide;
preferably, the oligonucleotide has a length ranging from 5 to 50 nt, more preferred 10-40 nt, most preferred 20-30 nt;
preferably, the oligonucleotide is dTs for separating a mixture of oligonucleotide with polyA tag, such as vitro transcribed mRNA bearing polyA; preferably, the length of said mRNA is of 30-4000 nt, preferable, 100-2000 nt.

In another preferred embodiment, the ratio of thickness of the shell layer to total thickness of the shell layer and the core layer is 0.5%-30%, preferably 1.0%-20%, more preferably 2.0%-15% and most preferably 3.0%-10%.

In another preferred embodiment, the thickness of the shell layer is 0.5-10 μm, preferably 1-8 μm, and more preferably 1.5-6 μm.

In another preferred embodiment, when the functional group of the core layer is the same to that of the shell layer, the functional group density of the core layer is D1, the functional group density of the shell layer is D2, and the chromatography medium has one of the following features:
1) D1/D2 is larger than 1.05, preferably 1.1, more preferably 1.5, and most preferably 2.0;
2) D2/D1 is larger than 1.05, preferably 1.1, more preferably 1.5, and most preferably 2.0.

In the second aspect of present invention, a synthetic polymeric porous mother medium is provided, wherein the mother medium is copolymerized from a monomer mixture which comprises:
(M1) at least a first monomer which is a crosslinking monomer;
(M2) at least a second monomer which comprises a monomer with a convertible functional group for hierarchical structure construction, and
(M3) an optional third monomer which has a special functional group for tuning chromatographic property.

In another preferred embodiment, the mother medium has one or more of the following features:
(a) specific pore volume in a range of 0.05-3.0 mL/g, preferably from 0.2 to 2.5 mL/g, most preferably from 0.4 to 2.0 mL/g;
(b) specific surface area in a range of 40-1200 m²/g, preferably from 60 to 1000 m²/g, most preferably from 80 to 800 m²/g;
(c) average pore size in a range of 30-5000 Å, more preferably from 50 Å to 3000 Å, most preferably from 100 Å to 2000 Å; and preferably, the average pore size is essentially homogeneous from inside to outside of the porous mother medium;
(d) volume average particle diameter (D₅₀) in a range of 1-1000 μm, more preferably from 1 μm to 500 μm, most preferably from 2 μm to 200 μm;
(e) particle size distribution (D₉₀/D₁₀) in a range of 1.0-2.2, preferably 1.0-1.5, more preferably 1.0-1.2, most preferably 1.0-1.05;
(f) alkene content of the mother medium in a range of 0.5-6.0 mmol/g, preferably 0.7-5.5 mmol/g, most preferably 0.9-5.2 mmol/g;
(g) the shape and/or form of the mother medium is a substantially flat particulate or monolithic rod or disk, the most preferred shape of a particulate is spherical or pseudo-spherical;
(h) the mother medium has the convertible functional group and/or the special functional group for tuning chromatographic property at the outside surface and inner portion thereof.

In the third aspect of present invention, a solid support is provided, wherein the solid support comprises:
1) the synthetic polymeric porous chromatography medium of the first aspect of the present invention or the synthetic polymeric porous mother medium of the second aspect of the present invention; and
2) a detectable label conjugated on the chromatography medium of the first aspect of the present invention or the other medium of the second aspect of the present invention.

In another preferred embodiment, the detectable label is selected from the group consisting of: protein, enzyme, catalyst, dye, fluorescent group, luminescent group, and combinations thereof;
preferably, the fluorescent group is selected from the group consisting of fluorescein (or FITC), Texas red, coumarin, rhodamine, rhodamine derivatives, phycoerythrin, Perci-P, EDANS, Congo red, and combinations thereof,
preferably, the luminescent group is selected from the group consisting of isoluminol, acridine, dioxetane, and combinations thereof.

In the fourth aspect of present invention, a method for preparing a synthetic polymeric porous chromatography medium of the first aspect of the present invention is provided, which comprises:
(a) providing a synthetic polymeric porous mother medium of the second aspect of the present invention;
(b) modifying the convertible functional group and/or the special functional group, thereby obtaining the synthetic polymeric porous chromatography medium of the first aspect of the present invention.

In another preferred embodiment, it comprises the following steps:
(Z1) providing a synthetic polymeric porous mother medium of the second aspect of the present invention;
(Z2) adding a modification reagent (such as a bromination reagent) to modify the convertible functional group of the mother medium, thereby obtaining an intermediate medium with chemically distinct two-layer structure, wherein the thickness of the shell layer is controlled by adjusting the adding amount of the modification reagent;
(Z3) modifying the resulting group(s) obtained in step (Z2), for example, by hydrolysis, to construct the shell layer of said medium with suitable binding functional group(s) depending on the separation needs;
(Z4) adding modification reagent(s) such as a bromination reagent to modify the convertible functional group(s) in the core layer of said intermediate medium;
(Z5) modifying the resulting group(s) obtained in step (Z4) with suitable ligand(s) to construct the core of corresponding intermediate medium with suitable binding functional group(s), thereby obtaining a chromatography medium with different binding functional groups inside and outside the chromatography medium or with same binding functional groups having a different density inside and outside the chromatography medium.

In another preferred embodiment, it comprises the following steps:
(Y1) providing a synthetic polymeric porous mother medium of the second aspect of the present invention;
(Y2) filling the inside of the mother medium with an inert filling;
(Y3) adding modification reagent(s) such as a bromination reagent to modify the convertible functional group outside the mother medium to obtain an intermediate medium with chemically distinct two-layer structure;
(Y4) modifying the resulting group obtained in step (Y3), for example, by hydrolysis, to construct the shell layer of said medium with suitable binding functional group(s) depending on the separation needs;
(Y5) removing the inert filling from inside of the mother medium;
(Y6) adding modification reagent(s) such as a bromination reagent to modify the convertible functional group inside the mother medium;
(Y7) modifying the resulting group obtained in step (Y6) to obtain a second binding functional group inside the mother medium, thereby obtaining a chromatography medium with different binding functional groups inside and outside the chromatography medium or with same binding functional groups having a different density inside and outside the chromatography medium.

In another preferred embodiment, the inert fillings are in liquid, gel/semi-solid or solid, regardless of their molecular weight and sizes; preferably, the inert fillings are in gel/semi-solid or solid form; most preferably, the inert filling is in solid form which will remain inside the pores throughout chemical transformations at the selective layers;
preferably, the inert fillings are 1-300 wt %, preferably 3-200 wt %, most preferably 5-150 wt % of mother medium;
and preferably, the inert fillings are not melt up to 200° C., preferably, the inert filling remain solid at 20° C.-150° C.

In the fifth aspect of present invention, a method for preparing the mother medium of the second aspect of the present invention is provided, which comprises the steps of:
(S1) providing a monomer mixture which comprises:
   (M1) at least a first monomer which is a crosslinking monomer;
   (M2) at least a second monomer which comprises a monomer with a convertible functional group for hierarchical structure construction, and
   (M3) an optional third monomer which has a special functional group for tuning chromatographic property; and
(S2) conducting a copolymerization process to obtain the mother medium of the second aspect of the present invention.

In another preferred embodiment, a porogen is used during the copolymerization process, and the method has one or more of the following features:
B1) the porogen is selected from the group consisting of hexanes, pentanes, octanes, pentanols, hexanols, heptanols, octanols, methyl isobutyl carbinol, cyclohexanol, toluene and xylenes, ethyl acetate, diethyl phthalate, and dibutyl phthalate, poly(propylene glycol), and poly(ethylene glycol);
B2) the weight ratio of total amount of porogens to total amount of monomers is 10%-400%, preferably 20-350%, more preferably 30-300%, most preferably 50-250%;
B3) the weight ratio of one single porogen to total weight of porogen is 0.1%-99.9%, preferably 1%-99%, more preferably 3%-97%, most preferably 5%-95%.

In another preferred embodiment, a swellable polymer/oligomer seed is used during the copolymerization process, and the method has one or more of the following features:
C1) the swellable polymer/oligomer seed is selected from the group consisting of (meth)acrylic, styrenic, oligostyrene, oligoacrylates, oligo-BMA, oligo-BA, vinyl acetate, and combinations thereof;
C2) the seed has a MW less than 70,000 g/mol for primary seed and 10,000 g/mol for later stage seed, more preferably less than 30,000 g/mol for primary seed and 5,000 g/mol for later stage seed.

In the sixth aspect of present invention, a chromatography method is provided, which comprises a step of using the chromatography medium of the first aspect of the present invention for selective separation of biomolecule(s).

In another preferred embodiment, the biomolecule is selected from the group consisting of lipids, proteins, antibodies, plasmids, RNAs, DNAs, VLPs, vaccines, viral vectors, viruses, bacteria.

In the seventh aspect of present invention, a liquid chromatography method for purifying and separating biologics is provided, wherein the method comprises the following steps:
1) providing a chromatography medium, biologics to be separated, first buffer, second buffer, and cleaning in place (CIP) solution;
wherein the chromatography medium is a synthetic polymer, has a porous structure, and has a 2-5 layered structures;
2) packing the liquid chromatography column with the chromatography medium, and the liquid chromatography column using the above method is obtained;
3) rinsing the liquid chromatography column with the first buffer;
4) loading the biologics to be separated into the liquid chromatography column obtained in step 3);
5) rinsing the liquid chromatography column obtained in step 4) with the second buffer, collecting the separated product to obtain the separated biologics;
6) rinsing the liquid chromatography column obtained in step 5) with the CIP solution, collecting the separated product, and removing the process-related impurities in the biologics.

In another preferred embodiment, the synthetic polymer is hydrophilic, preferably has a hydrophilic shell. It is not necessary homogeneously hydrophilic from outside to inside.

In another preferred embodiment, the porous structure is used for size exclusion separation; and
at least one inner layer and at least one outer layer of the chromatography medium have different types of binding functional groups, or at least one inner layer and at least one outer layer of the chromatography medium have the same type of binding functional groups with different binding densities, such that at least one inner layer and at least one outer layer of the chromatography medium have different chromatographic properties.

In another preferred embodiment, the chromatography medium has a core-shell structure.

In another preferred embodiment, the chromatography medium has one or more characteristics selected from the group consisting of:
1) the specific pore volume of the chromatography medium is 0.05 mL/g-3.0 mL/g;
2) the specific surface area of the chromatography medium is 40 m²/g-1200 m²/g;
3) the pore size of the chromatography medium is 30 Å-5000 Å; and preferably, the average pore size is essentially homogeneous from inside to outside of the porous mother medium;
4) the volume average particle size of the chromatography medium is 1 μm-1000 μm;
5) the particle size distribution (D₉₀/D₁₀) of the chromatography medium is 1.0-2.2.

In another preferred embodiment, the liquid chromatography column has one or more characteristics selected from the group consisting of:
1) the ion exchange equivalent of the liquid chromatography column chromatography medium in the core layer is 100-500 μmol/mL;
2) the linear flow rate of the liquid chromatography column is 10 cm/h-1000 cm/h;
3) the operating pressure of the liquid chromatography column is ≤100 bar.

In another preferred embodiment, the biologics to be separated is viral antigen selected from the group consisting of a virus, a viral vector, a vaccine, a virus-like particle, or a combination thereof.

In another preferred embodiment, in step 4), the loading amount of the biologics to be separated is in a range of 0.001-20 column volumes, preferably 0.1-15 column volumes, more preferably 1-10 column volumes.

In another preferred embodiment, in step 3-6), the flow rate of the liquid media (solution, buffer) is 10 cm/h-1000 cm/h.

In another preferred embodiment, in step 3-6), the operation pressure of the liquid media (solution, buffer) is ≤10 bar.

In another preferred embodiment, in step 6), the CIP solution is an aqueous based NaOH solution;
essentially free of organic solvent(s) such as ethanol, isopropanol, with the organic solvent concentration ≤5 wt %, preferably ≤1.0 wt %, more preferably ≤0.1 wt %;
preferably, NaOH concentration is in a range of 0.1-2.0M, preferably 0.2-1.5M, more preferably 0.5-1.0M;
preferably, purification temperature is in a range of 4-40° C., preferably 10-30° C., more preferably 15-25° C.

In another preferred embodiment, the biologics to be separated is antibodies selected from the group consisting of monoclonal antibodies, bispecific antibodies, multivalent antibodies, fragment antibodies, nanobodies, fusion proteins, antibody drug conjugates.

In another preferred embodiment, the biologics to be separated is mRNA.

In another preferred embodiment, the biologics to be separated is plasmids, RNAs, or DNAs.

In another preferred embodiment, the biologics to be separated is liposomes, extracellular vesicles, or exosomes.

In another preferred embodiment, the biologics to be separated is selected from the group consisting of lipids, proteins, antibodies, plasmids, RNAs, DNAs, VLPs, antigens, vaccines, viral vectors, viruses, bacteria.

In another preferred embodiment, the small molecule is a surfactant(s) and the large molecule is a protein; the surfactant is selected from polysorbates including Tween 20, 40, 60, and 80, polyethyleneoxide, poly(propylene oxide), sorbitan esters, ethoxylates, PEG, Poloxamer 188, Trion X-100, Trion X-114, Miglyol, and maltosides including n-dodecyl-β-D-maltoside (DDM), n-octyl-β-D-maltoside (ODM);

Prefer Tween 20, 40, 60, and 80, Poloxamer 188, Trion X-100, Trion X-114, Miglyol, n-dodecyl-β-D-maltoside (DDM), and n-octyl-β-D-maltoside (ODM).

In the first aspect of present invention, the invented synthetic polymeric porous medium with hierarchical multiple layer structure, defined as “core-shell(s)”, is composed of a central core and one or more concentric layers (shells) towards external geometry surface. It is more preferred to be two-layer core-shell structure.

In the second aspect of present invention, suitable polymerizable monomers carrying designed functional group(s) can be selected to either tune physicochemical properties of mother resins, or be used for further hierarchical structural modifications of mother medium synthesized.

In the third aspect of present invention, other than agarose, synthetic polymer-based matrix, with robust chemical/physical stability and promising physicochemical properties, were designed and developed through copolymerization of multiple monomers, such as (meth)acrylic, styrenic, and other vinylic monomers.

In the fourth aspect of present invention, an efficient process for producing synthetic polymeric mother resin with narrow size distribution and desired porous structure through sequential seeding processes was developed. Said mother resin can work as a platform for satisfying the requirements from systematic development of various LC technologies both in analytical and industrial fields, as well as offering solutions to such problems as limitation of scalability, lack of versatility of both resin chemistry and separation mode, and unsatisfied manufacturing efficiency. The core-shell construction combines two different functional groups to reach superior chromatography properties, which are difficult to be achieved by blending resins with single chemistry.

In the fifth aspect of present invention, resin chemistry, such as functional group(s) and group density in each layer, was developed and greatly expanded. In the simplest hierarchical structure, core-shell structure, the core and shell chemistry can be selected from abundant functional groups which can render such medium as SEC, SAX, WAX, SCX, WCX, HIC, affinity, or mixed-mode LC applications.

In the sixth aspect of present invention, the hierarchical structure modification was achieved through chemical kinetic/diffusion control of alkene modification such as bromination, wherein the controls of shell thickness and functional group density were also developed.

In the seventh aspect of present invention, the hierarchical structure modification was achieved through a masking-unmasking (protection-deprotection) process with inert fillings.

In the eighth aspect of present invention, said media can be applied as both batch mode and continuous mode, and can be physically packed into columns, discs, or other confined devices in any size to meet separation and purification requirements.

In the ninth aspect of present invention, said newly developed media, which combined size exclusion separation and various binding chemistry, were successfully applied for biomolecule purifications/separations, such as 1) separation and/or quantification of Tween 80 (small molecule), wherein new developed LC-MS columns with said media worked perfectly under non-denaturing conditions in a fast speed and high resolution. 2) VLP (large biomolecule) purification in an industrial scale, wherein high loading capacity, fast purification speed, streamlined downstream purification (DSP) process, high purification quantity such as purity and yield, and high throughput of DSP were achieved.

As for more detailed description of said achievements, the present invention disclosed three main achievements, aiming to be efficient, cost-effective, productive in both medium synthesis and separation applications, which include synthetic methodology of polymeric porous mother resin with narrow size distribution, modification methodology of medium with hierarchical structure and essentially homogeneous porous structure from inside to outside of the medium, and their applications in biomolecule separations.

In the present invention, the mother resin was synthesized through copolymerization of following multiple comonomers: at least one crosslinker monomer, at least one monomer with designed desired functional group(s), and optional monomer(s) that carry special functional groups to tune its properties, wherein said designed desired functional group(s) can be further used in hierarchical structure construction.

While synthesis of mother resin with narrow size distribution was realized through sequential seed polymerization processes via a shape template, using either low MW polymeric (or oligomeric) seeds or oil droplets, which are water insoluble, and swellable with monomer(s) used in later seed polymerization process.

One aspect of resin synthesis in the present invention, the resin size can be controlled through the following parameters, such as type and concentration of initiator, choice of water insoluble monomer(s), type and concentration of surfactant, stirring speed, size/shape/number/location of impeller, reactor geometry, seeds used in the immediate prior stage, as well as a swelling ratio (weight of monomers in the later seed polymerization vs. weight of said polymeric (or oligomeric) seeds). Through unremitting optimizations of reaction conditions, average particle diameter of said porous resins can be made with a desired size selected in a range of 1 μm to 1000 μm, it is more preferred from 1 μm to 500 μm, most preferred from 2 μm to 200 μm.

Meanwhile, particle size distribution (D₉₀/D₁₀), which was used to assess resin quality in terms of particle uniformity, for a medium made from conventional emulsion polymerization can be controlled to be ≤2.2, preferred ≤2.0, while for a medium made from seeding process can be controlled to be ≤1.6, preferred ≤1.5, more preferred ≤1.2.

Another aspect of resin synthesis in the present invention, the mother resin can be synthesized with desired pore structures, such as 1) pore size, wherein the pore size ranging from 30 Å to 5000 Å, more preferred from 50 Å to 3000 Å, most preferred from 100 Å to 2000 Å, can be turned and chosen based on the size of a target molecule in a specific application; 2) pore volume, which can be controlled in a range of 0.05 to 3.0 mL/g, preferred from 0.2 to 2.5 mL/g, most preferred 0.4 to 2.0 mL/g; 3) surface area, which ranges from 40-1200 m²/g, preferred from 60-1000 m²/g, most preferred 80-800 m²/g.

The present invention discloses different types of resins made from said mother resins above, varying in particle size, pore size, hierarchical structure, ligand, and ligand density. Said hierarchical structure medium refers to a separation resin with at least two layers, where the core refers to the most inner layer, while the shell(s) refer(s) to the outer layer(s) from the core. One of the preferred said hierarchical structures is core-shell two-layer structure with distinct chemical functional groups or same functional group with different density. Said suitable functional group(s) which could render such layer LC separation mechanism chosen from size exclusion chromatography (SEC), strong anion exchange (SAX), weak anion exchange (WAX), strong cation exchange (SCX), weak cation exchange (WCX), hydrophobic interaction chromatography (HIC), affinity, or mixed mode, as shown in FIG. 1.

Said different types of media in the present invention, such as (meth)acrylic, styrenic, and other vinylic based polymeric media, show robust physical and chemical stability, it can be used in various operational conditions, such as pH in a range of 1-14, temperature with a tolerance limit up to 200° C., various aqueous or organic solvents, pressure with a tolerance limit above 200 bars, autoclave, etc.

Concept, “segregated chemistries”, was reported in U.S. Pat. No. 5,522,994, and Science 1996, 273, 205-211. The authors disclosed a pore-size-specific modification: two kinds of chemistry were “strictly segregated in pores of different sizes”. Resins with segregated chemical functional groups can be prepared through pore size specifically, by using chemical reagents or enzymes with different molecular size relative to the pore size according to. Thus, steric effect will differentiate the pores of different sizes, resulting in preferential modification of some pores with certain size from others which gave mixed functional properties at different pores. Polymers of large size can block the small modifying reagent to penetrate into micropores by steric hindrance, therefore these modifying reagents can only modify the larger pores.

In said research, the concept, “hierarchical layer” or “core” or “shell”, was not discussed or clarified. Meanwhile, no experimental data can support said structure of “layer”, or “core” and “shell”. In contrast, in the present invention, said “core”, “shell” structure with distinct functional group(s) was well defined and visualized spatially.

The preparation of “segregated chemistries” at small pores and large pores of porous resin with steric control of chemical reagents and enzymes is only of limited usage due to the scarcity of suitable chemical reagents and enzymes for the preparation of the resins with different pores.

As known, chemical modifications with smaller chemical reagents as described in detail offer much wider diversity to introduce the chemical functional groups at either inner pores or outer layers of porous resins, which can thus provide multimode chromatographic media with any combination of distinct chemistries in principle. The present invention discloses two synthetic methodologies for construction of core-shell structure, which are chemical kinetic control and masking-unmasking (protection-deprotection) processes, as shown in FIGS. 5, 6, and 8.

Said core-shell structure modification in the present invention was first achieved through chemical kinetic/diffusion control of partial bromination of allyl group, such bromination via kinetic control has been applied to the agarose-based beads with swellable pores, according to U.S. Pat. No. 10,493,380. However, more rigid resins with well-defined pore structure and pore size distribution are important to good chromatographic media. Hence, such (meth)acrylic, styrenic, and other vinylic polymeric media will be a top choice for such resins. Establishment of core-shell structure construction of these resins is a great expansion to create a repertoire of rigid chromatographic media to meet the purification needs.

Additional benefit of this invention over U.S. Pat. No. 10,493,380 results from the precise control on average bead size and size distribution. Applying a similar approach (bromination via kinetic control) to those mother resins of Capto™ Core with polydispersity bead size in nature as shown in FIG. 2D and Table 2, shell thickness uniformity and volume fraction of core layer vs. shell layer will vary between large beads and small beads. In the present invention, beads with narrow size distribution tend to render more uniform shell thickness and similar level of volume fraction of core layer and shell layer in the whole bead population.

In process, fast chemical transformation of said allyl group was affected with bromine, at outer layer resin surface before bromine diffuses onto inner or deeper pore surface, which can then be modified further with bromine at the 2^{nd} time combining with different transformation. Thus, this two-step modification results in distinct chromatographic properties at different layers, as shown in FIG. 6.

We examined the possibility of whether we can apply said core-shell modification concept to beads reported in the above literature (U.S. Pat. No. 5,522,994, and Science 1996, 273, 205-211). However, core-shell structure construction of a similar resin composed of the same monomers in said reports, Generik MC60 from Sepax Technologies, Inc., via chemical kinetic control of epoxide group was not successful, even though various nucleophiles were tried, such as amines, and thiols. Possibly, the reactivity of epoxide was not sufficient enough to be transformed simultaneously by addition of such nucleophiles, which resulted in failure of core-shell construction.

As known, the functional group density, a very important performance factor of separation medium, is kind of challenging to be controlled in the later modification processes in case of additional process steps and their associated high manufacture cost. While in the present invention, the desired functional group of said mother resin used for ligand modification, is from polymerizable monomer(s), and shows relatively high group density, which offers a board space to control ligand density of corresponding separation medium.

As shown in Table 2, the allyl groups, from the copolymerized monomer, show high density in a range of 1.0-5.1 mmol/g, the density of separation ligand(s) derivatized from allyl group can be controlled in a broad range, from 1 to 800 μmmol/mL, upon a LC separation request, here units of ally content, μmmol/mL and mmol/g, are convertible according to their corresponding volume and dry weight relationship. While Capto™ Core resins give a range from 40-80 μmol/mL. Meanwhile, the hydrophilicity of said shell or core is tunable through chemical modification using hydrophilic agents, such as 2-hydroxyethanethiol, rac-3-sulfanylpropane-1,2-diol, Dextran, etc.

Meanwhile, in said chemical kinetic/diffusion process, the shell thickness of said medium was tunable upon the amount of bromine used in partial bromination step, as shown in FIG. 7 and Table 3.

An alternative core-shell modification of porous agarose resin can also be enhanced according to U.S. Pat. No. 7,208,093. According to the pattern, inner layer/pore can be blocked with inert solvent, thus shielding the inner pore surface or deeper layer surface from chemicals reactive to otherwise the entire resin surface or slow down the chemical modification on the inner pore surface. Once the outer layer was chemically modified by first reagent, the solvent was removed. Further modification on outer layers can be brought upon with the inner pore remaining intact. Desired chemical modification can proceed at inner pore surface with the same 1^{st} reagent or different 2^{nd} reagent. The resin so prepared will have two different chemical functional groups at inner layer and outer layer surface.

The present invention encompasses another process to prepare chromatographic medium which are of two or more phase-chemistries on single porous polymer or non-polymer bead. The process is termed as masking-unmasking (protection-deprotection). The entire pore or partial but deeper inner pore surface was protected or masked, with inert filling, followed by 1^{st} chemical transformation at shallow pore surface or resin surface other than inside protected pore surface. Then the inert filling was removed to expose the pore surface or deeper pore surface. A 2^{nd} chemical transformation can be affected at these unmasked surfaces. Thus, the process will furnish two or more layers of chemical functionalities on a single porous resin, as shown in FIGS. 8 and 9.

Said hierarchical construction methodologies can be appliable to other support matrix modified with designed active functional group(s), such as allyl, vinyl groups, etc. Said matrix can be organic or inorganic materials, such as agarose, cellulose, dextran, chitosan, and their derivatives, silica and its derivative silica, glass, zirconium oxide, graphite, tantalum oxide etc. Said chromatography medium can be physically converted/transformed into LC columns or devices for molecules separation and purification.

The present invention also discloses the separation applications of said medium, with core-shell two-layer structure and designed pore size, which is used for analytical and industrial separations. Said medium combines size exclusion separation and binding chemistry, where larger molecules/organism/particle, such as cell, cell particles, bacteria, virus, virus like particle, plasmids, antibodies, proteins, are excluded from the no-binding shell and analyzed or collected in a flow-through mode, while smaller molecules, such as DNA, DNA fragments, RNA, small virus, small proteins, cell lysis, amino acid, surfactants, etc., penetrate and temporarily trap/bind into the functionalized core of the separation medium, which can be eluted later for analysis or collection. Said separation can be done with single column or with multiple columns (with continuous chromatography). The practice can be accomplished with the batch mode beside the conventional binding and elution mode.

Here the separation sample comprising at least two substances with distinguished MW. MW ratio M1/M2≥2; preferably M1/M2≥5, most preferably M1/M2≥10, where M1 refers to the largest substance, and M2 refers to the smallest substance in the separation mixture.

For example, Resin 23 platform can be used to separate biomolecules from the surfactants, such as Tween 20, 40, 60, and 80, which is often used in stabilizing biotherapeutic formulation. Such preferred biomolecules can be therapeutic proteins with MW range of 10 KDa to 3 MDa, as shown in FIG. 15.

Resin 29 can be applied to separate mixtures of VLP, vaccine, viral vector or virus, from smaller molecules such as oligonucleotides, impurity of host cell proteins and endotoxins. Preferred VLP, vaccine, viral vector or virus are of particle sizes in the range of 10-1000 nm, most preferred particle size is 20-1000 nm, as shown in FIG. 16.

Protein A, protein G, or protein L can be conjugated to the core of said resin for purification of mAb from smaller proteins or fragments via binding to Fc and/or Fab domains. For example, Resin 49 with Protein A ligand was successfully applied to purify a sample of antibody fermentation broth in high yield, as shown in FIGS. 17 and 18.

Another preferred type of biomolecules is oligonucleotide with poly A tag, such as in vitro transcribed mRNA bearing poly A tail. Here A refers to adenine. The length of said mRNA is of 30-4000 nt, preferred 100-2000 nt. Said oligonucleotide carries 10-100 Å tag, most preferred length is 10-30 nucleotides. Resin 45 with 25 dT ligand was successfully applied to purify a sample of crude mRNA in high yield, as shown in FIG. 19.

Overall, this present invention provides a platform resin solution to many LC challenges as disclosed in the Background Section: 1) Bead size, porous structure and pore wall functional group density can be predetermined and such properties are tunable based on application needs. 2) Versatile medium chemistry. 3) Monomers are readily available, well characterized and monomer properties are well controlled. 4) A bead of a defined pore size, bead size, and bead chemistry can be made commercially available in large quantity in a short period of time. 5) The core-shell construction method combines two different chemistries to reach superior properties, which are difficult to be achieved by blending resins with single chemistry or using the “segregated chemistries” approach. 6) Using a same type of polymer resin can make a process transfer streamlined from analytical characterization to production, and from small scale to industrial scale production. 7) Bead platform is versatile and accommodating. A customized resin with special properties can be developed in a short period of time. 8) Abundance of possible new LC applications.

In this invention, it should be clarified that, 1) “Medium”, “media” used here is a general term for a solid porous support with any suitable shape and shape for LC applications. Includes but not limited to substantially flat particulate, a monolithic rod and disk and covers spherical or pseudo-spherical particulate. 2) “Resin”, “particle”, “bead” and “seed” refer to a subgroup of “medium”, “media”, that is spherical or pseudo-spherical particulate as the same. 3) “Mother”, “base”, and “raw” medium refer to porous medium as synthesized without any chemical modifications. 4) “Intermediate” medium refers to a medium partially chemically modified with surface functional groups which can be used directly or be further chemically modified to the final or finished beads. 5) “Final”, “finished” medium refers to a medium fully chemically modified, which can be used in LC separation applications.

### BRIEF DESCRIPTION OF THE DRAWINGS

FIG. 1. Scheme of chromatography medium structure, FIG. 1A. a hierarchical synthetic polymeric porous resin with Core-Shell (two-layer) structure, FIG. 1B. a hierarchical synthetic polymeric porous resin with a hydrophilic shell and an alkylamine core.

FIG. 2. SEM of mother beads. All are synthetic polymer resins except for FIG. 2D.

FIG. 2A. Polydispersed EGDMA-AMA-GMA, 35.1 μm (D₅₀) (Resin 1)

FIG. 2B. Polydispersed EGDMA-AMA-GMA, 53.8 μm (D₅₀) (Resin 2)

FIG. 2C. Monosized EGDMA-AMA-GMA, 59.1 μm (D₅₀) (Resin 13)

FIG. 2D. Capto Core 700, agarose-based core-shell resin, 88.3 μm (D₅₀) (Comp. resin 1).

FIG. 2E. Water Oasis HLB resin, conventional porous structure, 26.8 μm (D₅₀) (Comp. resin 2).

FIG. 3. SEM of mother beads with different choices of monomers (3A-3D) and of final finished beads cross-sectioned (3E-3F).

FIG. 3A. Monosized EGDMA-AMA-GMA, 23.8 μm (D₅₀) (Resin 6)

FIG. 3B. Monosized EGDMA-AMA, 29.2 μm (D₅₀) (Resin 9)

FIG. 3C. Monosized DVB-AMA, 13.0 μm (D₅₀) (Resin 18)

FIG. 3D. Monosized DVB-AMA-PVP, 48.8 μm (D₅₀) (Resin 20)

FIG. 3E. Monosized EGDMA-AMA-GMA, 56.6 μm (D₅₀) with a hydroxy shell and a butylamine core (Resin 29). Right image is the zoom-in region of the left image as indicated by a white frame. The imaging area is chosen to illustrate porous structure in both the core layer and the shell layer.

FIG. 3F. Monosized EGDMA-AMA-GMA, 56.6 μm (D₅₀) with a hydroxy shell and a butylamine core (Resin 29). Right image is the zoom-in region of the left image as indicated by a white frame. The imaging area is chosen to highlight interior porous structure and exterior porous structure in the shell layer.

FIG. 4. Particle size distribution of monosized Resin 5 (D₅₀: 28.7 μm)

FIG. 5. Multilayer (core and one shell shown in this scheme) modification methodologies: chemical kinetic control method and masking-unmasking (protection-deprotection) method.

FIG. 6. Multilayer (core and one shell shown in this scheme) modification process via chemical kinetic control.

FIG. 7. FTIR monitors of allyl group intensity against amount of Br₂ addition based on Resin 5.

FIG. 8. Multilayer (core and one shell shown in this scheme) modification via masking-unmasking (protection-deprotection) method.

FIG. 9. FTIR monitors of wax and allyl intensity through the transformation process based on Resin 9.

FIG. 10A. Visualization of core-shell hierarchical structure: CLSM studies of Resin 46 labeled with EDANS.

FIG. 10B. Visualization of core-shell hierarchical structure: CLSM studies of Resin 84 labeled with EDANS.

FIG. 11A. Visualization of core-shell hierarchical structure: CLSM studies of Resin 47 labeled with Congo red.

FIG. 11B. Visualization of core-shell hierarchical structure: CLSM studies of Resin 73 labeled with hydroxy in the core and with Congo Red dye in the intermediate layer and EDANS in the outer layer.

FIG. 12A. Comparison of IEC (gray) and NaNO₂ retention time (black) among Resin 28, 29, 31 and Capto Core 700.

FIG. 12B. Comparison of IEC (gray) and NaNO₂ retention time (black) between Resin 23 and Oasis MAX.

FIG. 13. Comparison of Erbitux NSB studies between Resin 23 and Oasis MAX.

FIG. 14. Comparison of trap capacity of Tween 80 between Resin 23 and Oasis MAX at breakthrough point.

FIG. 15. Chromatograms of Tween 80 and Erbitux mixture under non-denaturing conditions using a column packed with Resin 23.

FIG. 16. Process chromatogram of a crude VLP sample (80-90 nm diameter) using a column packed with Resin 29.

FIG. 17. Domains of rSPA (native recombinant Staphylococcal Protein A ligand) and rSPAc is rSPA with a terminal cysteine.

FIG. 18. Process chromatogram of a crude mAb purification in the capture step using a column packed with Resin 49.

FIG. 19A: Process chromatogram of a crude mRNA (˜1000 nt, made from IVT upstream process) by using Resin 45.

FIG. 19B: Fractionation analysis.

FIG. 20: Process chromatogram of a crude bacteriophage sample in the capture step using a column packed with Resin 31.

FIG. 21: Process chromatogram of an Adeno virus sample in the polishing step using a column packed with Resin 31.

FIG. 22: Process chromatogram of a crude inactivated flu vaccine sample in the capture step using a column packed with Resin 29.

FIG. 23: Process chromatogram of a crude a plasmid sample in the capture step using a column packed with Resin 29.

### DETAILED DESCRIPTION OF THE INVENTION

This invention provides a synthetic polymeric porous chromatography medium with hierarchical multiple layer structure and essentially homogeneous porous structure from inside to outside of the medium, whose multiple layers are covalently modified with distinct chemical functional groups or same functional groups with different density. The chromatography medium is made from a mother resin through polymerization of multiple monomers: at least one crosslinking monomer, at least one monomer with designed functional group further used in hierarchical structure construction, and optional monomer(s) carrying special functional groups to tune its properties. The mother resin is successfully obtained with narrow size distribution and desired porous structure in the presence of at least one porogen, at least one initiator, and at least one surfactant.

In the present invention, said monomers can be selected from any one or more of the following agents, including acrylate, acrylamide, ethylene terephthalate, ethylene, propylene, styrene, vinyl acetate, vinyl chloride, vinyl pyrrolidone, and their derivatives, etc. One preferred monomer is “(meth)acrylic” based compounds. Another preferred monomer is “styrenic” based compounds including unsubstituted (styrene) and substituted (α-methylstyrene, ethylstyrene). Monomers sufficiently insoluble in water (≤10 g/L) are preferred to construct a polymeric porous medium with sufficient mechanical strength.

As used herein, term “crosslinking monomer” or “crosslinker” herein refers to a polymerizable monomer that carries multiple polymerizable functional groups. Crosslinker monomers are allyl, or vinyl derivatized with carbon-carbon double bonds, including di-, tri-, and multiple vinyl-aromatic compounds, di-, tri-, and multiple (meth)acrylate compounds, and di-, tri- and multiple vinyl ether compounds, such as divinylbenzene (DVB), ethylene glycol dimethacrylate, pentaerythritol dimethacrylate, pentaerythritol triacrylate, pentaerythritol tetraacrylate, sorbitol dimethacrylate, poly(ethylene glycol) diacrylate, poly(propylene glycol) diacrylate, trimethylolpropane triacrylate, bis2-(methacryloyloxy)ethyl phosphate, N, N′-methylenebisacrylamide, glycerol 1,3-diglycerolate diacrylate, 3-(acryloyloxy)-2-hydroxypropyl methacrylate, 1,5-hexadiene, allyl ether, diallyl diglycol carbonate, di(ethylene glycol) bis(allyl carbonate), ethylene glycol bis(allyl carbonate), triethylene glycol bis(allyl carbonate), tetraethylene glycol bis(allyl carbonate), glycerol tris (allyl carbonate), ethylene glycol bis(methallyl carbonate), diallyl phthalate, triallyl isocyanurate, diallyl isophthalate, diallyl terephthalate, diallyl itaconate, diallyl 2,6-naphthalene dicarboxylate, diallyl chlorendate, triallyl trimellitate, triallyl citrate, 2,4,6-Triallyloxy-1,3,5-triazine, 1,3,5-Triacryloylhexahydro-1,3,5-triazine, glyoxal bis(diallyl acetal), N,N-diallyldimethyl ammonium salts, ethylene glycol diallyl ether, etc.

While said optional monomer(s), which can tune any property of mother resin, can be any one or more of the following, such as glycidyl methacrylate, 2-hydroxyethyl methacrylate, 2-carboxyethyl acrylate, 2-hydroxyethyl acrylate, 2-hydroxyethyl methacrylate, methyl methacrylate, methacrylic acid, hydroxypropyl methacrylate, 2-(methacryloyloxy)ethyl acetoacetate, mono-2-(methacryloyloxy)ethyl maleate, benzyl acrylate, butyl acrylate, styrene, DVB, N-vinylpyrrolidone, etc.

One important aspect of present invention, the chemical or physical property of said mother medium is tunable through choosing variety of monomers carrying desired functional group(s), and adjusting the ratio of monomers, such as hydrophilicity, hydrophobicity, hydrogen bonding, affinity, π-π interaction, electrostatic force, and Van der Waals force, etc.

In the present invention, said mother medium should contain at least one inactive, less active, or protected functional group(s) which can survive during polymerization process, and then further be used in hierarchical modification directly or indirectly. Said functional group(s) can be amino, sulfenyl, benzyl, phenyl, alkyl, alkynyl, hydroxyl, carboxyl, aldehyde, halo, sulfonyl groups, etc. In the preferred embodiment, the reactive groups are allyl groups, vinyl groups, or other alkenyl groups with carbon-carbon double bonds, such as alkenyl group from allyl (meth)acrylate, vinyl (meth)acrylate, diallyl maleate, DVB, 1,3,5-trivinylbenzene, etc. Said alkenyl group can also be selected from any suitable crosslinking monomer as described above. The most preferred monomers are allyl methacrylate and diallyl maleate.

This invention describes an “emulsion polymerization” process to create synthetic polymeric mother medium, which is formed in the presence of porogens, initiator, and surfactant through polymerization of multiple monomers. Herein “emulsion polymerization”, “microemulsion polymerization”, “suspension polymerization”, and “dispersion polymerization” can be used interchangeably. Monomers and porogens were dispersed or emulsified as small oil droplets into a continuous medium, usually water. At least one oil-in-water surfactant was used to stabilize oil droplets and particles during their formation. As least one initiator was used to initiate polymerization. Emulsion polymerization can be carried out using continuous, batch, and semi-batch process. Emulsified monomer, porogen, initiator can be charged into a polymerization using one feed stream or two. Porogens usually stay with monomers, and oil-soluble initiator can stay by itself or with part of monomers. Polymerization starts when reaction temperature reaches to 20-40° C. which is below the one-hour half-life temperature of the initiator. Monomers are gradually microphase separated from porogens to form a crosslinked polymeric matrix.

In the present invention, “porogen” or “pore forming reagent” is generally chosen from thermodynamically poor solvents (precipitants), thermodynamically good solvents, or oligomers which are at least partial soluble to one monomer. Soluble oligomers in principle can be treated as a high MW solvent. So swellable low MW oligomer seeds or a low Tg polymeric seeds can be consider as a high MW polymeric solvent. So herein “solvent”, or “porogen solvent” covers convention solvents and polymeric solvents. It is believed without binding to any theory, that the porous structure of a resin such as pore size, surface area, pore volume, pore connectivity, surface pore morphology (smooth vs. rough) is controlled by the choice(s) of porogens, and ratio of total amount of porogens to total amount of monomers, and weight ratio of a specific porogen. For example, polymeric porous resins formed from a poor solvent (or large total amount of porogens) tend to have large average pore size, low surface area, low pore volume, and rough surface porous morphology. On the other hand, polymeric porous resins formed from a good solvent (or small total amount of porogens) tend to have small average pore size, high surface area, high pore volume, and smooth surface porous morphology. In practice, usually a pair of good porogen and poor porogen can be chosen to balance porous structure properties and mechanical strength needed as a LC medium.

In the present invention, polymerization process and pore formation process are carefully modulated which results in essentially homogeneous porous structure from inside to outside of the porous mother medium.

One or more suitable porogens are selected from one or more linear and branched (C4-C10) alkanols, one or more linear and branched (C4-C16) alkane, aromatic hydrocarbon, alkyl esters, aliphatic ketones, aromatic ketones, oligomer of alkyl oxide like PPG and PEG. Conventional solvent(s) can be selected from hexanes, pentanes, octanes, pentanols, hexanols, heptanols, octanols, methyl isobutyl carbinol, cyclohexanol, toluene and xylenes, ethyl acetate, diethyl phthalate, dibutyl phthalate, PPG, PEG or any of their mixtures. Polymeric solvent(s) from swellable seeds can be selected from (meth)acrylic, styrenic, and other vinylic monomers such as oligostyrene, oligoacrylates, oligo-BMA, oligo-BA, vinyl acetate or any of their mixtures. The ratio of total amount of porogen to total amount of monomers is 10%-400%, preferred 20-350%, more preferred 30-300%, most preferred 50-250%. If a pair of porogens is chosen, the weight ratio of any porogen to total weight of porogens is 0.1%-99.9%, preferred 1%-99%, more preferred 3%-97%, most preferred 5%-95%.

One-hour half-life temperature of “Initiator”, free-radical initiator, is preferred to be 55-110° C., more preferred 60-105° C., most preferred 65-100° C. It can be chosen from inorganic peroxides, such as persulfate salts, organic peroxides, such as tert-butyl peroctoate, benzoyl peroxide, lauroyl peroxide and azo type initiators, such as azobisisobutyronitrile (AIBN). It is believed that choice, amount, and reactivity of an initiator could play a significant role in porous structures.

“Chain-transfer reagent” can effectively terminate a growing polymer chain, so it can be used to control average MW of seeds in addition to the use of initiator. Chain-transfer reagent can also increase consistence of seed preparation. Suitable chain-transfer reagents include halomethanes, disulfides, thiol (as known as mercaptans). Linear or alicyclic alkyl thiols, aromatic thiols, or thioglycolic esters with C2-C12 are preferred, C3-C10 are most preferred.

“Surfactant”, “dispersant”, “suspending agent”, or “stabilizer” is a processing aid in emulsion polymerization which can be neutral or carry charges, such as small soap molecules, or oligomer and polymers carrying polar moiety. They generally have a hydrophilic moiety that favors contacting water and a hydrophobic moiety that favors contacting water insoluble monomer/porogens. They tend to stay at the interface of hydrophilic phase and a hydrophobic phase and be compatible both phases and thus stabilize emulsion polymerization. They can be selected from common soap molecules, celluloses, hydroxyalkyl cellulose, polyvinylpyrrolidones, polyvinyl alcohols, PEG, PPG, PPG/PPG copolymers and their mixtures. Polymeric stabilizers help control bead size and bead size distribution and increase lot-to-lot consistency through controlling viscosity of a polymerization system.

This invention also describes a process to make a monodispersed porous mother bead, wherein bead size distribution is controlled and realized through sequential seed polymerization process using a low MW polymeric (oligomeric) seeds or water insoluble oil droplets which are swellable with monomer(s) used in later seeding process. Said low MW polymeric (oligomeric) water insoluble seeds made in a suspended solution can be used in-situ for the next stage seeding process. Alternatively, a primary seed if made into a high Tg polymer (well above rt, like ≥40° C.) can be separated, collected, stored, and redispersed to make a seed solution, and then used thereafter.

The size of seeds is controlled by the polymerization parameters (initiator type and concentration, choice of water insoluble monomer(s), surfactant type and concentration, stirring speed, size/shape/number/location of impeller, and reactor geometry) for primary seeds and weight ratio of the monomers in the later phase seed polymerization to early phase polymeric (oligomeric) seeds.

Alternatively, a suspended solution of oil droplets (seeds) containing solvent and/or oleophilic monomer(s), which are both water insoluble, can be produced through a mechanical way by forcing the mixture above, water and suitable surfactant through a well-defined porous solid support like membrane (including vibrational “jetting” and natural “jetting”), or porous glass “filtration”, or small orifice (single discrete or in array form). The size of oil droplets (seeds) can be controlled by amplitude of mechanical disruption and choice of oil molecules (intrinsic viscosity, MW, surface tension). This approach was not pursued in the present invention due to its inefficiency for making seeds with small particle size.

Water insoluble (less than 1 wt % solubility in water) monomer(s) can be one or more monomer that are soluble in each other and can form a later stage seed without inducing a macro-phase separation. Choice of monomers can be water insoluble acrylate or vinyl monomer with up to 2 wt % crosslinker like diacrylate or divinyl monomer. Preferable, no crosslinker is used in making seeds in all stages. The preferred monomers are benzyl methacrylate, butyl acrylate, styrene, or their binary/tertiary mixture. Seeds can be pre-swelled with a desired solvent, which is soluble with seeds but less soluble or insoluble with water (less than 1 wt % solubility in water), to enhance seeds swellability.

The seeds are liquid or gel in nature at rt (a temperature between 0° C. and 40° C.), and should have a low MW if constituent monomers can form a high Tg polymer (well above rt, like ≥40° C.). MW is less than 70,000 g/mol for primary seeds and 10,000 g/mol for later stage seeds; more preferred MW is less than 30,000 g/mol for primary seeds and 5,000 g/mol for later stage seeds. However, MW requirement is not restricted as the above for a low Tg polymer (around or below rt, like ≤40° C.). Under both conditions, seeds can be swelled by monomers, porogens or an optional solvent used in later stage seed polymerization process.

Excessive initiator (inorganic peroxide, organic peroxide, azo type initiators), excessive chain transfer reagent (thiol, thiol ether, thiol ester containing molecules), or their combination is used to keep seeds MW low and enable high swellability of such seeds. Keeping seeds MW low is essential if constituent monomers can form a high Tg polymer (well above rt, like ≥40° C.). >0.5 wt % initiator and/or >1 wt % chain transfer reagent with respect to weight of polymerizable monomer(s) is preferably used. More preferred to have >2 wt % initiator and/or >3 wt % chain transfer reagent.

Seed size and porous bead can be predefined by swelling ratio, weight of monomer(s), porogen(s) and pre-swelling solvent if selected to weight of seeds, assume macrophase separation does not occur during seed polymerization. The swelling ratio in each seed polymerization step is preferred to be 2-300, more preferred 5-200, even more preferred 10-100, most preferred 20-80. The size of seed and porous bead can be built bottom up through a successive sequential seed polymerization process.

The definition of resin size distribution and its measurement method can vary significantly among resin manufactures. It is a challenging task to compare resin size and size distribution based on a reported value on resin COA or specifications sheet. In the present invention, we want to define particle (resin or seed) size and particle size distribution as described below, volume average particle diameter (D₅₀), and particle size distribution (D₉₀/D₁₀) were used to assess resin quality in terms of particle uniformity. The narrower particle size distribution, the smaller value of D₉₀/D₁₀. For a prefect monodispersed system, D₉₀/D₁₀ is 1.0. While for polymeric particles made through conventional emulsion polymerization, D₉₀/D₁₀ is generally larger than 2.0 before any sizing/sieving. Here we loosely define “monodispersed”, “monodispersity”, as D₉₀/D₁₀ in a range of 1.0-1.1, “monosized”, “narrowly dispersed” and “substantially uniform” as D₉₀/D₁₀ in a range of 1.0-1.5, while “polydispersed” and “polydispersity” as D₉₀/D₁₀≥1.5.

Bead size can be controlled and tuned through stirring speed, type and concentration of surfactant, size/shape/number/location of impeller, reactor geometry in a conventional polymerization, as well as seed size and concentration, and swelling ratio in seed polymerization.

Through the synthetic strategies described above, porous mother resin with desired size diameter, porous structure, functional group(s), and group density was successfully achieved as design upon separation requirements. As shown in Tables 1 and 2, the beads properties are summarized as, 1) Pore volume, ranging from 0.05 to 3.0 mL/g, preferred from 0.2 to 2.5 mL/g, most preferred from 0.4 to 2.0 mL/g. 2) Surface area, ranging from 40 to 1200 m²/g, preferred from 60 to 1000 m²/g, most preferred from 80 to 800 m²/g. 3) Pore size, ranging from 30 Å to 5000 Å, more preferred from 50 Å to 3000 Å, most preferred from 100 Å to 2000 Å. 4) Volume average particle diameter (D₅₀), ranging from 1 μm to 1000 μm, more preferred from 1 μm to 500 μm, most preferred from 2 μm to 200 μm. 5) Particle size distribution (D₉₀/D₁₀), wherein, D₉₀/D₁₀≤2.2, preferred ≤2.0, for a medium made from conventional emulsion polymerization, and D₉₀/D₁₀≤1.6, preferred ≤1.5, for a medium made from seed polymerization process. 6) alkene content, ranging from 0.5 to 6.0 mmol/g, preferred from 0.7 to 5.5 mmol/g, most preferred from 0.9 to 5.2 mmol/g.

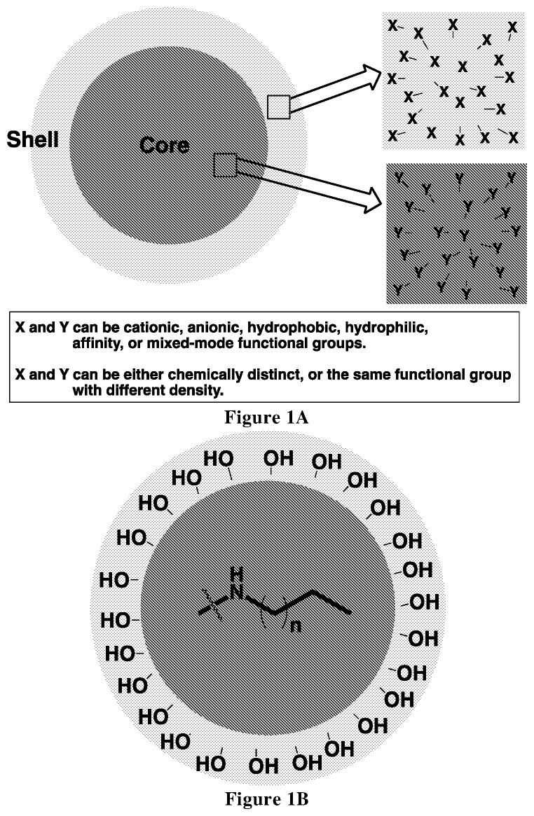

The present invention demonstrates various microporous polymeric medium with hierarchical structure and essentially homogeneous porous structure from inside to outside of the medium for LC separation. Here said hierarchical structure indicates the medium has at least two layers, where the core refers to the most inner layer, while the shell(s) refer(s) to the outer layer(s) from the core, where each layer of the medium either have the same functional groups with different density, or distinct functional group(s), as shown in FIG. 1 and Tables 3-4.

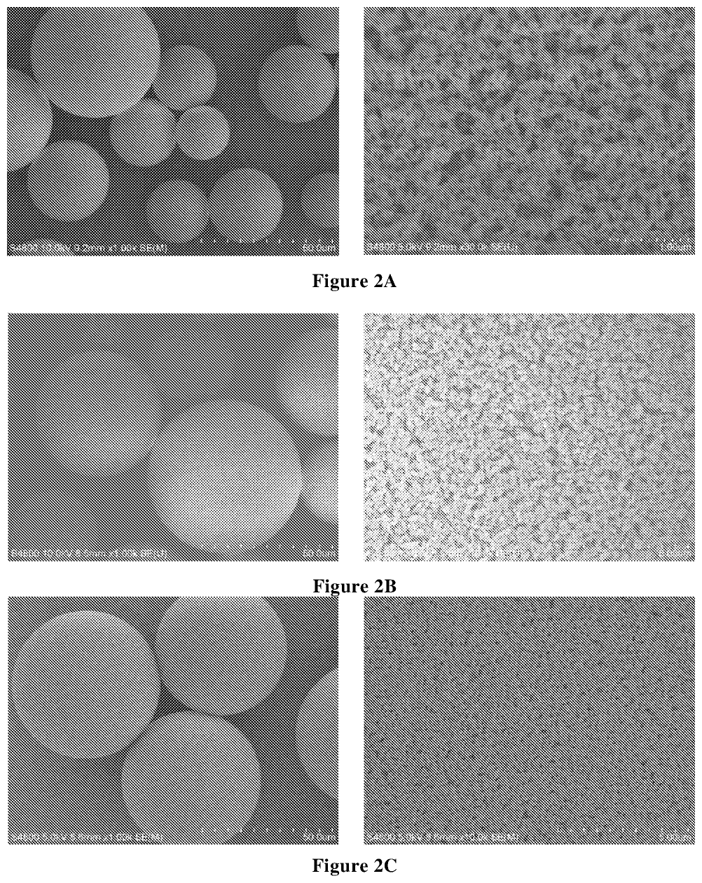

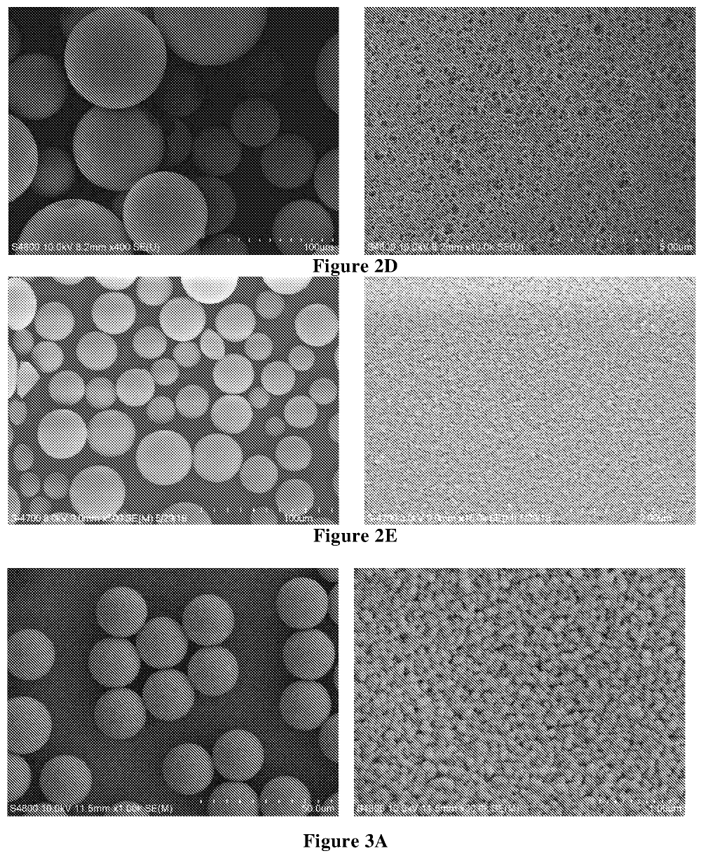

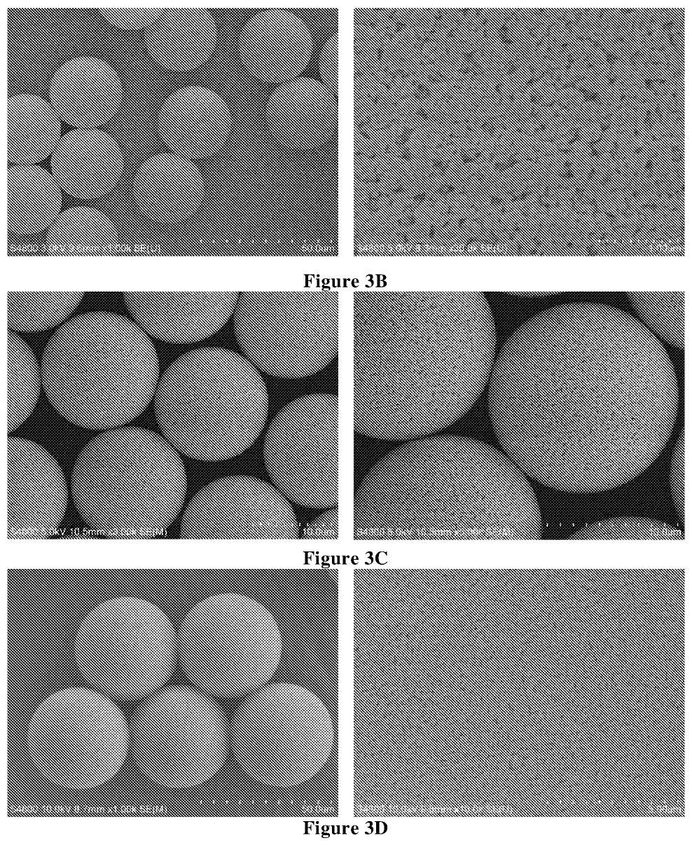

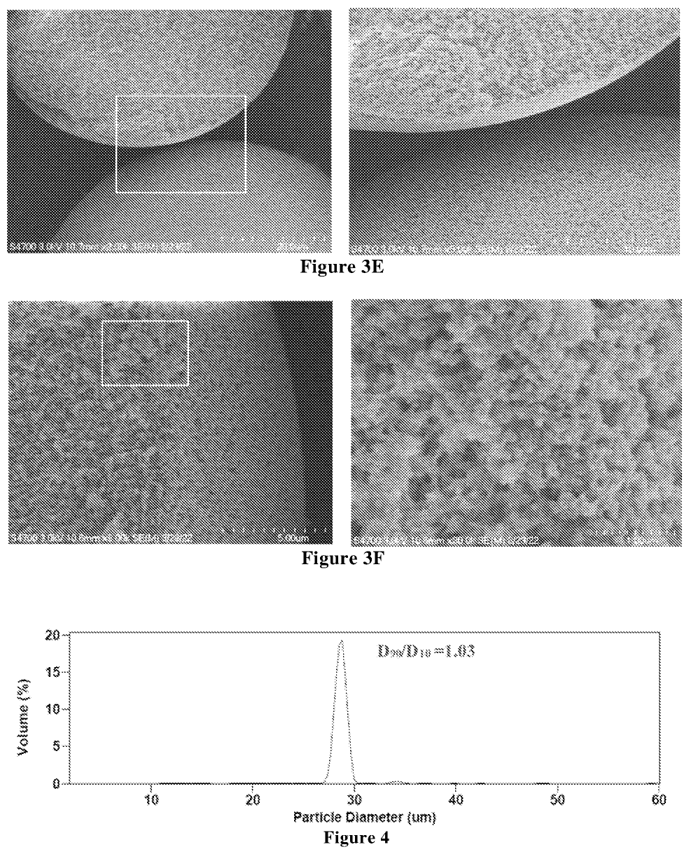

In the present invention, mother medium has an essentially homogeneous porous structure from inside to outside. Due to the much smaller size of ligand in the core and shell compared to average pore size of said medium, and the very mild surface modification conditions, the porous structure of the final beads with newly modified core-shell structure can be retained homogeneously as mother beads show. The homogeneous porous structure of the core-shell structural final beads in terms of pore size and pore density were visualized through by cross-sectioned SEM, as shown in FIGS. 3E and 3F. There is: 1) essentially no difference between the core and shell layers in FIG. 3E; and 2) essentially no difference between interior porous structure and exterior porous structure in the shell layer in FIG. 3F. However, as shown in FIG. 2D, Capto™ Core 700 beads show relative open in the core layer and relative tight in the shell layer in term of said porous structure.

The choice of different ligands on either shell or core depends on the chromatographic separation requirements, which can be classified as following:
(I) Cationic ligands used for anion exchange chromatographic separation mode: the pendant cationic or ionizable groups can be any suitable cationic groups, such as ammonium, sulfonium, phosphonium, or other groups, preferable amino groups including preliminary amines, secondary amines (such as ethylamine, butylamine, hexylamine, octylamine, etc.), tertiary amines (dimethylamine, diethylamine, etc.), and quaternary amines (trimethylamine (TMA), N, N-dimethylbutylamine (MBA), etc.).
(II) Anionic ligands used for cation exchange chromatographic separation mode: the pendant anionic or ionizable groups can be any suitable anionic groups, such as sulfonate (—SO₃H), phosphonate (—PO₃H), carboxylate (—CO₂H), and their derivatives.
(III) Hydrophobic ligands used for hydrophobic interaction chromatographic separation mode: the pendant hydrophobic groups be any suitable hydrophobic group linked to the backbone through oxygen atom (O), nitrogen atom (N), sulfur atom (S), ether, ester, or amide groups, such as linear or branched alkyl chain (C1-C18), oligo(ethylene oxide), phenyl, benzyl groups, and their derivatives.
(IV) Mixed-mode ligands used for multiple interaction chromatographic separation mode: the pendant mixed-mode activated ligands groups, which have a stationary phase ligand composed of at least one hydrophobic moiety at peripheral position or branch position, and at least one ionic or ionizable groups at peripheral position or branch position, or embedded in the hydrophobic moiety, can be any suitable mixed-mode activated ligands. Here the ligands can enhance the purification ability of biomolecules or others that are difficult to separate by other chromatography methods. Suitable ligands can be selected from alkylamine such as ethylamine, butylamine, hexylamine, and octylamine, etc., N-Benzyl-N-methylethanolamine (BMEA), N, N-Dimethylbenzylamine (DMBA), N, N-Dimethyl(2-phenoxyethyl)amine, 2-(pyridin-4-yl)ethanethiol, 3-Phenylpropan-1-amine, 2-benzamido-4-mercaptobutanoic acid (BMBA), etc.
(V) Affinity ligands used for affinity chromatographic separation mode: the pendant affinity activated ligands can be any one of suitable affinity active pairs, which can bind or interact to its ligand/binding partner, such as Protein A, Protein G, Protein L, phenyl boronic acid, dT (T refers to thymine), and sense/antisense oligonucleotide, etc. Said ligand can also be iminodiacetic acid (IDA), tri(carboxymethyl)ethylene diamine (TED), nitrilotriacetic acid (NTA), and other metal chelating ligands.
(VI) A mixture of ligands having different functionalities for mixed-mode like chromatographic separation: due to the high reactivity of bromohydrin/epoxide to various nucleophiles, two or more ligands can be linked to the beads via one-shot or stepwise manner, which gives the separation matrix with diversified stationary phases. Here the combination can be RP/IEX, HILIC/IEX, or RP/HILIC. The choice of ligands can be any suitable one or more mentioned above.
(VII) polyT ((poly(dT)n), and peptide (such as protein A).

In another preferred embodiment, in the present invention, the molecular weight of the ligand is less than 1000, preferably less than 500, preferably less than 300, preferably less than 150, preferably 40-150.

In another preferred embodiment, in the present invention, the ligand does not include polysaccharide, especially modified or unmodified dextran.

In the present invention, due to the small molecular weight of the ligand, the pore size in the core layer and the pore size in the shell layer in the synthetic polymeric porous chromatography medium are basically the same.

The present invention demonstrates various types of chromatography medium with hierarchical multiple layer structures and essentially homogeneous porous structure from inside to outside of the medium, as illustrated in FIG. 1. One of the preferred said hierarchical structures is core-shell two-layer structure with chemically distinct functional groups, where said medium is composed of hydrophilic inert shell, and ligand-activated core, as shown in Tables 3-4. On the other hand, the invention also discloses a different medium composed of an ionic or ionizable shell, and suitable hydrophilic/ligand-activated core, such as Resins 50-53, and 55. Here in the most advantageous embodiment, the charged pendant groups on the shell are of the same charge as that of compounds that are undesired in a chromatographic process. For example, Resin 53 with negatively charged sulfonate group (—SO₃H) in the shell was designed to repel cells and cell debris in the process for separation of protein from a cell lysate.

The active ligands mentioned above can be linked to the backbone directly through covalent bond, and preferably, carbon-nitrogen bond, carbon-oxygen, and carbon-sulfur bonds. Spacer arms can also be included between the backbone and the active ligands, which can help the active ligands to separate their spatial charge and/or increase opportunity to interact with isolation materials such as proteins, amino acids, nucleic acids, and DNA molecules. The spacers groups which can enhance biomolecule binding capacity and/or selectivity should be used. In certain embodiments, the spacer group includes one or more moieties selected from the groups consisting of alkylamido, alkylsulfide, hydroxyalkyl, alkylamino, hydroxyalkylaminoalkyl, hydroxyalkylaminoalkyl hydroxyalkyl, alkylaminoalkyl, etc. The spacer arms can of any suitable length with linear, branched, or combinations thereof.

The layer thickness in the present invention is controlled and can be tuned. The ratio of thickness of the shell layer to total thickness of the shell layer and the core layer is 0.5%-30%, preferably 1.0%-20%, more preferably 2.0%-15% and most preferably 3.0%-10%.

The functional group density of each layer is controlled and can be tuned independently. In one case the functional group of the core layer is the same to that of the shell layer, the functional group density of the core layer is D₁, the functional group density of the shell layer is D₂, and the chromatography medium has one of the following features:
1) D₁/D₂ is larger than 1.05, preferably 1.1, more preferably 1.5, and most preferably 2.0;
2) D₂/D₁ is larger than 1.05, preferably 1.1, more preferably 1.5, and most preferably 2.0.

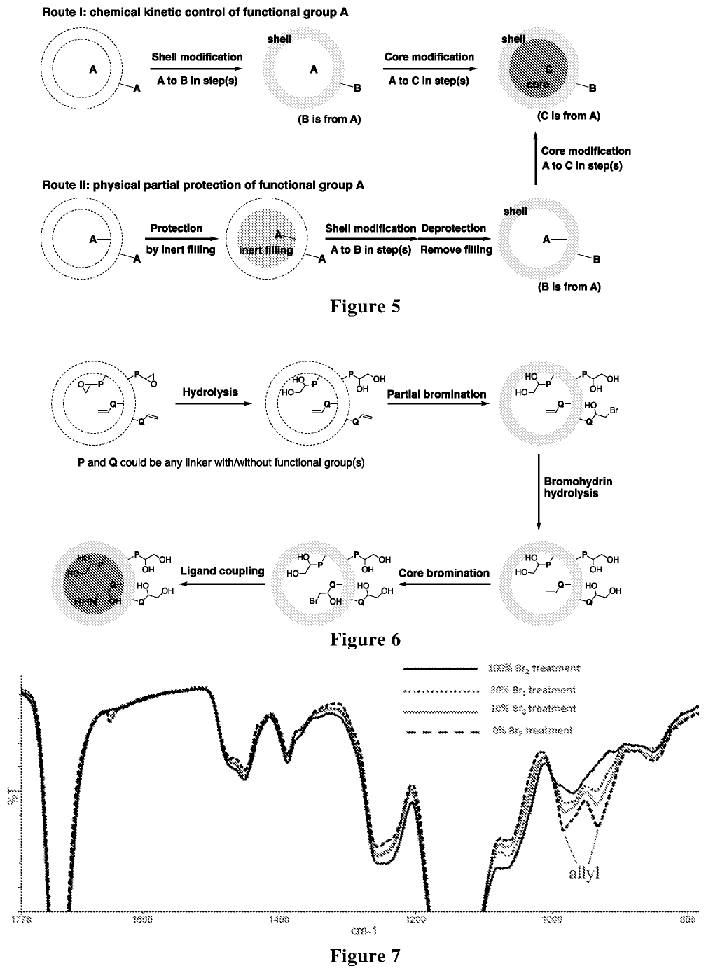

The present invention provides two synthetic routes/methodologies to construct core-shell hierarchical structure by using allyl contained mother resins, as shown in FIGS. 5, 6, and 8.

One of the successful core-shell modification examples was achieved through chemical kinetic control of allyl bromination by using Resin 13. As illustrated in FIG. 6: step 1 was designed for improving hydrophilic property of mother beads through hydrolysis of epoxide groups; step 2, which was partial bromination of allyl groups in the outer layer of the resin, and step 3, which was hydrolysis of the resulting bromohydrin groups, were key steps for creating hydrophilic shell, here the pendant hydrophilic groups can also be derivatized from 2-hydroxyethanethiol, 3-sulfanylpropane-1,2-diol, Dextran, etc., then the core was modified with various ligands through either amination or thioetherification in step 5, after allyl bromination in the core in step 4. Here allyl group, in the core of said intermediate resin in step 3, can also be transferred into epoxide group for the following ligand coupling use.

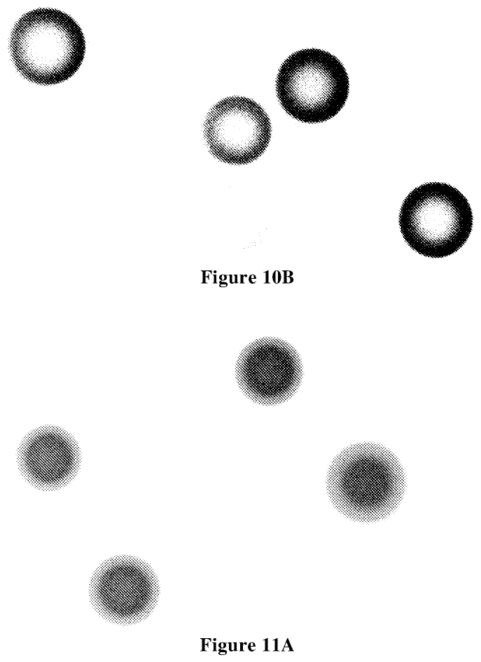

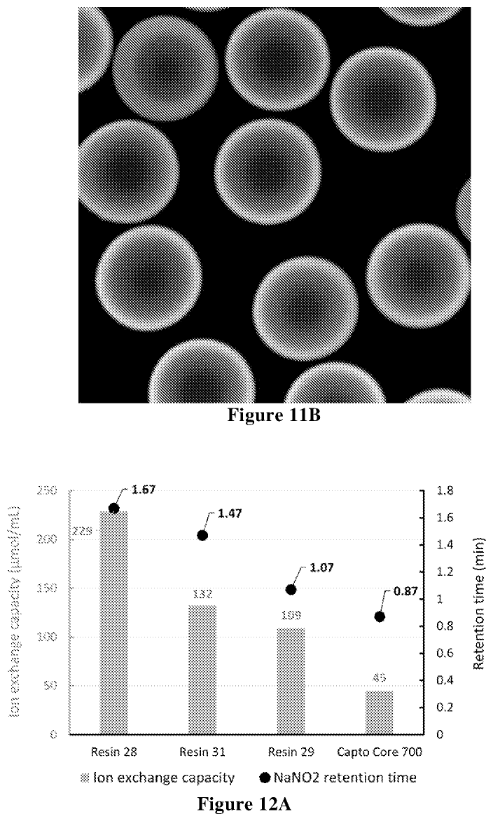

It is very challenging to directly visualize the resulting core-shell structure. Through a few campaigns on exploring and optimizing experimental conditions, direct evidence of core-shell structure was obtained from confocal microscopy. For an illustrative purpose, Resin 46 and Resin 84 labeled with EDANS, and Resin 47 labeled with Congo Red dye in their cores were design and synthesized. Here an intermediate resin GM1C with hydroxylated shell and brominated core was selected for the core labeling, because the amino group of either EDANS or Congo Red can selectively react with bromohydrin in the core other than hydroxyl group in the shell of said intermediate resin. The core-shell two-layer structure was clearly visualized through confocal laser scanning microscopy (CLSM, LSM 880) studies, as shown in FIG. 10A and FIG. 11A. Meanwhile, said fluorescently labeled resin also illustrates prospects of fluorescent materials with hierarchical structure, which can be applied in material science.

Resin 73 labeled with hydroxy in the core and with Congo Red dye in the intermediate layer and EDANS in the outer layer was design and synthesized. The core-shell three-layer structure was clearly visualized through confocal laser scanning microscopy (CLSM, LSM 880) studies, as shown in FIG. 11B. The outer layer was modified with EDANS through sequential partial bromination/amination process, and Congo red dye modification in the middle layer was achieved through repeating this process following through procedure GM1t. Meanwhile, said resin is labeled with two chemically distinct fluorescent dyes, and each dye could be placed in a layer of choice (for example dye 1 in layer 1 and dye 2 in layer 2 vs. dye 2 in layer 1 and dye 1 in layer 2). This illustrates prospects of fluorescent materials with hierarchical structure and with multiple fluorescent responses, which can be applied in material science.

The present invention showed that the shell thickness can be controlled as designed through controlling amount of bromine used into the partial bromination step. For example, the shell thickness of Resin 5 can be controlled to be 1.7 μm with 0.3 equivalents of bromine addition, or 0.5 μm with 0.1 equivalents of bromine addition, as shown in Table 3. These results were evidenced through FT-IR studies, which showed that the intensity of allyl groups decreased upon stepwise addition of bromine, as shown in FIG. 7.

For supporting said shell thickness control, a reliable alkene titration method was also developed by using AgNO₃, which was used for tracing molal weigh of bromine reacted with alkene of said resin, the results of alkene content were recorded for the corresponding resins in a dry form. As shown in Table 2, the resins new developed show relatively high alkenyl group density in a range of 1.0-5.1 mmol/g, which offers a board space to control ligand density of corresponding separation medium, as discussed below.

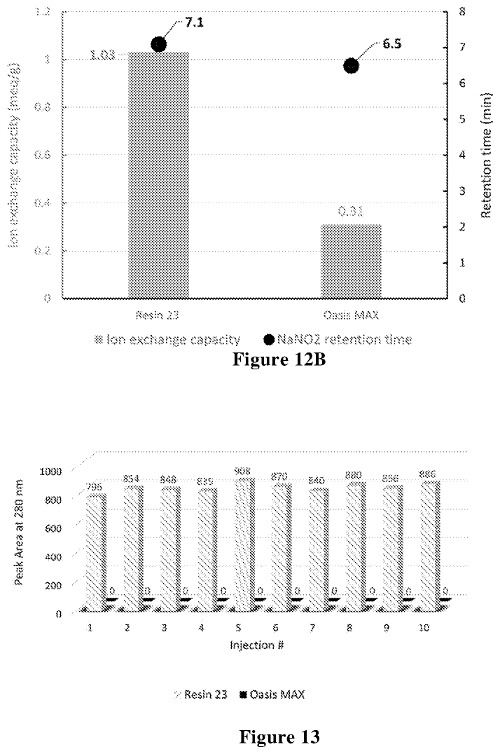

The present invention here provides a methodology to control the functional group density in the shell or core. Since bromohydrin is reactive to various nucleophiles, competitive nucleophiles can be used to compete with desired ligands in reacting with bromohydrin which can control the ligand loading capacity, while competitive nucleophiles chosen here should not play negative role in LC separation. For example, ion exchange capacity (IEC) of Resin 28 produced by reacting with excess amount of butylamine in DMF/H₂O mixture was determined to be 229 μmol/mL, while IEC of Resin 29 and 31 were determined to be 109 and 132 μmol/mL, respectively, if 1 equivalent and 5 equivalents of butylamine were used in alkaline DMF/H₂O mixture, respectively, as shown in Table 3 and FIG. 12A. Here the IEC values of separation medium were determined via amine titrations by using AgNO₃, which was used for titrating chlorine ion (Cl⁻) from the pretreated ammonium chloride salt produced by said resin and aqueous HCl.

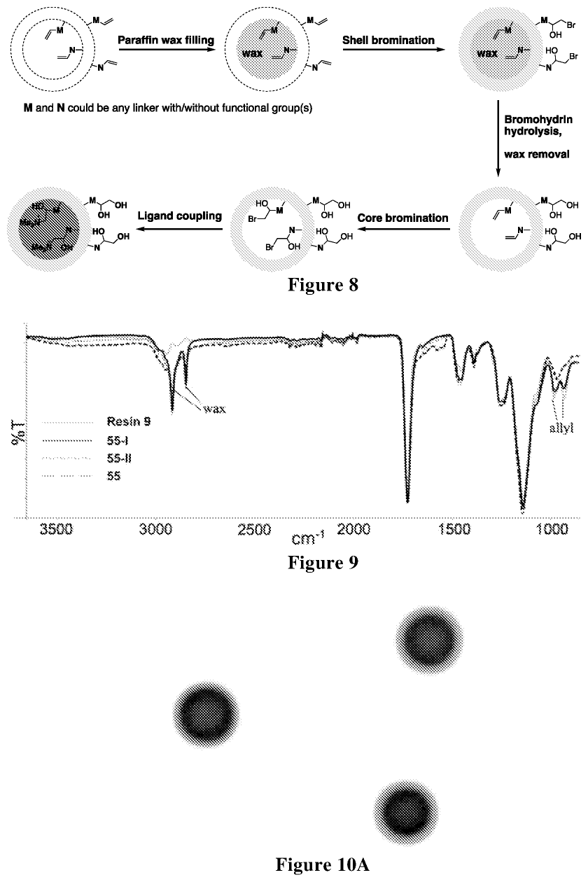

Because the surface chemistry at the inner/deeper pore surface is different from the surface outside/at shallower of the pores, an alternative way of core-shell modification was also achieved through masking-unmasking (protection-deprotection) process, as shown in FIG. 8.

In this process, the entire pore or partial but deeper inner pore surface can be protected or masked, with inert fillings, followed by chemical transformation at resin surface other than inside protected pore surface, or shallow pore surface. After the chemical modification was completed at shallower pore surface or none pore surface, the inert filling can be removed to expose the pore surface or deeper pore surface, further chemical transformation can be carried out at these unmasked surfaces. Thus, the process will furnish two or more layers of chemical functionalities on a single porous resin. Here FT-IR monitors were done during this modification process, as shown in FIG. 9.

Resin 54-59 and 84 with said core-shell structure were successfully achieved through allyl bromination, as shown in Examples 62-67 and 102, and Table 4. It is worth making an additional statement of Generic MC resin with epoxide group in core-shell construction through said masking-unmasking process, even though various reaction conditions were tried, such as inert fillings, temperature, control of pH, reaction agents, etc., the results were not satisfying due to the poor stability and pH sensitivity of epoxide group. Meanwhile, epoxide has limited reactivity, which restricted its broad application in said core-shell modification processes.

Here said inert fillings can be hydrophobic compounds, hydrophobic polymers, hydrophilic compounds, hydrophilic polymers in solid, gel/paste or liquid forms. Said inert fillings can be natural or synthetic. Choice of the inert fillings will depend on the hydrophilic/hydrophobic characters of the porous resins. The hydrophobic compounds, hydrophobic polymers will be used toward hydrophobic porous resins, while hydrophilic or hydrophilic polymers can be used for the porous resins with hydrophobic surface. The inert filling will remain inside the pore or on the deeper inner pore surface through the intended chemical transformations. Here said inert fillings will not participate the chemical transformations brought upon the exposed resin surface.

Said solid inert fillings can be completely dissolved/dispersed in a solvent at rt or desired elevated temperature. Resin is uniformly dispersed in this solution of inert fillings. The solvent is removed by rotavapor or lyophilized to produce dry resins with inert fillings attached.

The reaction solvents used in the processes ensure the inert fillings remain inside the pores or on the inner pore surface throughout the desired chemical transformations. The solvents can be aqueous or commonly used organic solvents or mixture of aqueous and organic solvents. Inert fillings are not soluble or barely soluble in the reaction solvents under the reaction conditions, which will promote the desired chemical transformation on the said resins. The removal of inert fillings or deprotection can be affected after the subsequent desired chemical transformation completed or simultaneous with the subsequent desired chemical transformation as long as the once protected surface area remains intact.

The inert fillings can be 1-300% weight of porous resin, preferably 3-200%, most preferably 5-150%, depending on the pore wall surface area which needs to be protected.

The present invention discloses that the shape and form of said chromatography medium can be selected from a range of options depending on the application requirements. The surface of said medium can be substantially flat or planar, rough, or patterned, and alternatively can be rounded or contoured. Exemplary contours that can be included on a surface are wells, depressions, pillars, ridges, channels or the like. Said medium can be selected from bead, box, column, cylinder, disc, dish (e.g., glass dish), fiber, film, filter, membrane, net, pellet, plate, ring, rod, roll, sheet. It is preferred the substrate is a substantially flat particulate, a monolithic rod and disk. Most preferred shape of particulate is spherical or pseudo-spherical.

Said chromatography medium can be physically converted/transformed into LC columns or other confined devices for molecular separations and purifications. Specifically, LC columns or devices can be analytical columns, guard columns, preparative columns, semi-prep columns, HPLC columns, UPLC columns, UHPLC columns, FPLC columns, flash columns, gravity columns, capillary columns, spin columns, disposable columns, monolithic columns, extraction cartridges and plates, etc.

While said column or devices can combine with batch-mode and continuous mode such as counter current chromatography. Said column can be used as single column or multi-column format in continuous or non-continuous (conventional) chromatography. Said column can be applied to flow-through mode or bind-elute mode in the analytical or industrial purification processes.

In said column, ID can vary from 0.1 millimeter to 2 meters and any length from 1 millimeter to 2 meters. Said column, or disc housing materials can be stainless, PEEK, glass or borosilicate glass, or other synthetic polymeric materials such HDPE (high density polyethylene).

The present invention also provides successful LC applications in biomolecule separation. Said core-shell two-layer medium with hydrophilic character in outer layer and IEX in the inner layer, such as Resin 23, can be applied to separate the biomolecules from surfactants which is often used in stabilizing biotherapeutic formulation, as shown in FIG. 15. Such separation is critical in quantitively analyzing the composition of formulated biotherapeutics. Said surfactants can be nonionic detergent, such as Tween 20, 40, 60, and 80, polyethyleneoxide, poly(propylene oxide), sorbitan esters, ethoxylates, PEG, Poloxamer 188, Trion X-100, Miglyol, maltosides including n-dodecyl-β-D-maltoside (DDM), n-octyl-β-D-maltoside (ODM).

Nonionic surfactants are commonly used in the formulation of therapeutic monoclonal antibodies (mAb) to prevent protein denaturation and aggregation. Tween 20 is a complex mixture of esters of different polymeric polar head groups and various fatty acid tails with multiple degrees of esterification. The polysorbate structural heterogeneity, complicated by the presence of proteins at high concentration, makes the characterization of polysorbate highly challenging in protein formulations. It is also critical to understand the molecular heterogeneity and stability of Tween 20 in mAb formulations as polysorbate can gradually degrade in aqueous solution over time by multiple pathways, which cause losing surfactant functions and leading to protein aggregation. Polysorbate degradation is dependent on pH and temperature of the solution. Therefore, there are increasing interests to identify, quantify Tweens and associated molecules from different commercial sources and/or from different degradation pathway.

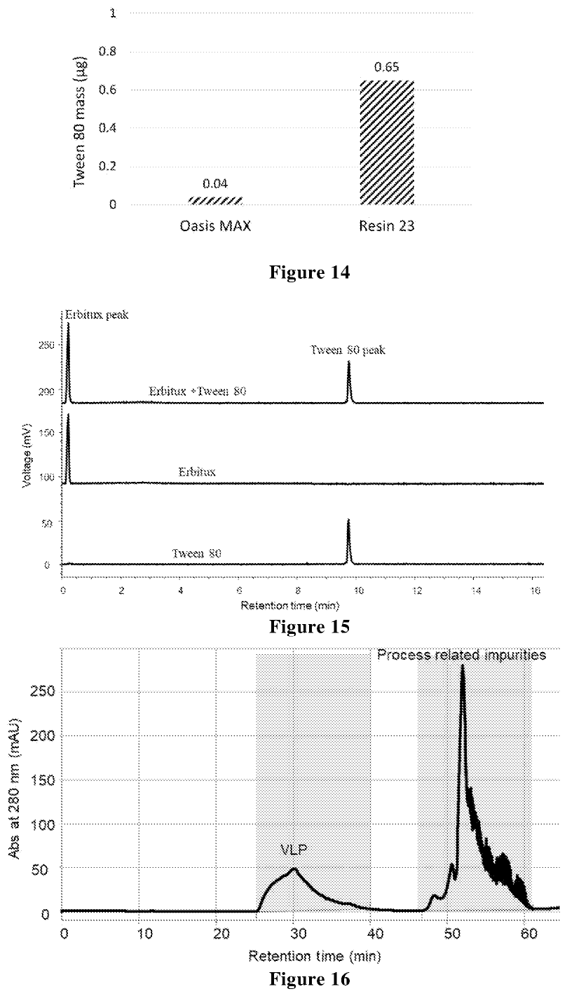

Said Resin 23 has high functional group density in the core, which was determined quantitatively by amine titration (1.03 meq/g vs 0.31 meq/g of Oasis-MAX resin), and qualitatively by comparison of column retention time of NaNO₂, as shown in FIG. 12B. This result was satisfactorily evidenced by Tween trapping capacity study, here the breakthrough of Tween 80 was observed to be 0.65 μg, which showed the capacity was 16 times higher than that of Oasis-MAX media (0.04 μg) under the same conditions, as shown in FIG. 14.

Nonspecific binding (NSB) study on the column packed with said resin indicated all Erbitux can be recovered through a flow-through mode, while all 10 Erbitux injections sticked in the column packed with Oasis-MAX resin probably due to hydrophobic interaction, as shown in FIG. 13.

In this present invention, LC columns packed with Resin 23 and alike show superior and unique performance for separating nonionic detergent, such as Tween 20, 40, 60, and 80, polyethyleneoxide, poly(propylene oxide), sorbitan esters, ethoxylates, PEG, Poloxamer 188, Trion X-100, Miglyol, and maltosides including n-dodecyl-β-D-maltoside (DDM), n-octyl-β-D-maltoside (ODM). The new LC columns have also shown benefits over incumbent columns like Waters' Oasis-MAX in many aspects. Waters' Oasis-MAX resin is chemically homogeneous and does not have a core-shell hierarchical structure. The column developed in this invention 1) can cover several application needs while Oasis-MAX column just covers single application; 2) performs a fast analysis of Tween titer determination and identification (Example 69 and 70), since the packing resin has minimal non-specific binding and a unique hierarchical structure of a hydrophilic shell and a hydrophobic core; 3) allows using LC-MS compatible buffers so that it eliminates a buffer exchange step which cannot be avoided by using many other analytical columns 4) can operate in protein native buffer condition (non-denaturing conditions) and allow analysis on both protein and Tween (Example 72); 5) can effectively trap (remove) Tween from a biologics formulation and has a higher binding/trapping capacity compared to Waters' Oasis-MAX column (Example 68 and 71); 6) could be readily expanded to analyze other nonionic surfactants or even ionic surfactants since the packing resins developed in this present invention could have tunable properties through adjusting chemical core-shell modifications.

Another advantageous application of said medium can be applied to separation of biomolecules mixture in such that the outer layer of the porous resin will exclude or prevent large cells, VLPs, vaccines, viral vectors or viruses, or liposomes or LNP (lipid nanoparticles) to interact with functional groups in the inner pore space, via ionic, affinity, HIC or mixed ionic/HIC mode, while smaller impurities such as DNA, RNA, oligonucleotides, endotoxin, other small proteins and peptides can be absorbed on the inner resin pore surface, which later can be washed off with high salt eluent or CIP reagent such as 0.5-1.0 M NaOH aqueous based solution with or essentially without using an organic solvent.

As an example, Resin 29 was successfully applied in purifying VLP, as shown in FIG. 16. In the purification process, a VLP, about 80-90 nm across, was excluded from the shell and collected as flow-through, while most process related impurities were temporarily absorbed by multimodal ligand (butylamine), and then removed from the beads by CIP procedures.

Monoclonal antibodies (mAbs) and antibody fragments (Fabs and ScFv) today represent the majority of biotherapeutic market. Their purification is greatly advanced with the development of different affinity ligands which will selectively bind to different domains of mAbs such as Fc and/or Fab domains, Fabs and ScFv. Produced recombinantly, purification of proteins via affinity chromatography is becoming a well-established platform technology. Nevertheless, the separation of mAb assembly such as bispecific mAb from smaller fragments or constituents remains challenging.

Resin 49, constructed with Protein A conjugated onto the inner layer while the outer layer being hydrophilic, can be applied to separate the mAb or Fc containing protein from smaller fragments. The fully assembled mAb or Fc containing protein can be collected as flow through, while smaller fragments were absorbed to the inner core.

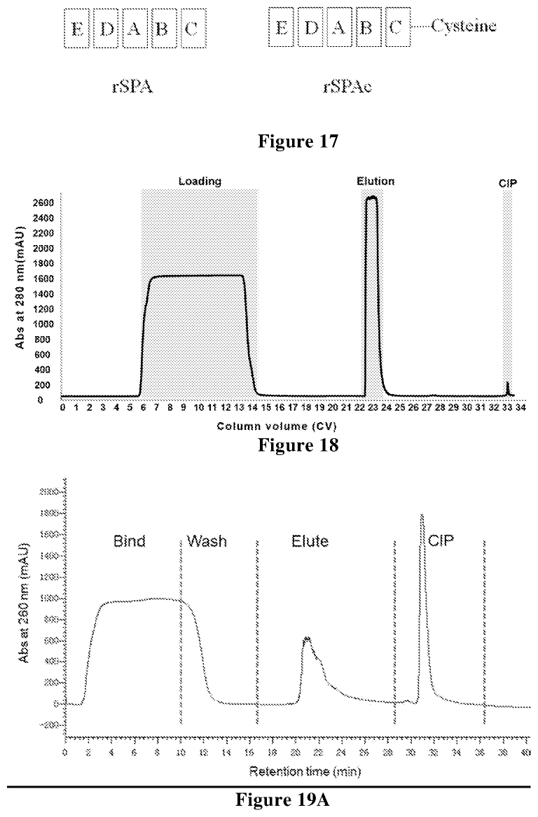

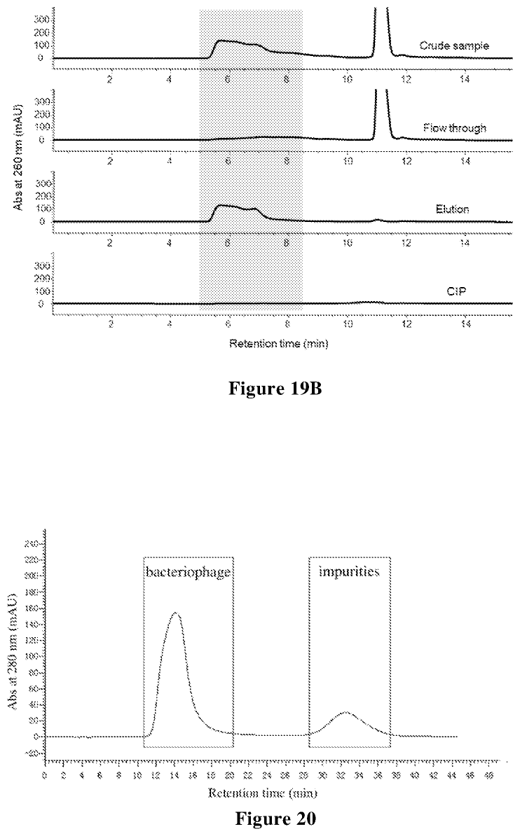

Such protein A can have domains and sequences selected from rSPA (native recombinant Staphylococcal Protein Aligand, U.S. Pat. No. 5,151,350) or rSPAc as shown in FIG. 17. Such rSPA or rSPAc can be produced recombinantly from E. coli. and rSPA is commercially available from Repligen (PN: 10-2001-XM). rSPA or rSPAc was conjugated to said resin via lysine as multipoint attachment via cysteine as single point attachment. The conjugation can be affected with or without extra spacer 2-20 atoms containing carbon, nitrogen, oxygen and sulfur or a combination of these atoms. Successful purification application of a sample with antibody through Resin 49, which was designed with an affinity ligand, protein A, was shown in FIG. 18. In this process, desired antibody was recovered in 95% yield with a purity of 97%.

Such Protein A can be mutants of rSPA or rSPAc with increased/prolonged alkaline stability at 0.1M NaOH or 0.5M NaOH or LOM NaOH. Such Protein A and its mutants can be produced recombinantly from E. coli.). Such Protein A and its mutants can be conjugated to said resin via lysine as multipoint attachment via cysteine as single point attachment. The conjugation can be affected with or without extra spacer 2-20 atoms containing carbon, nitrogen, oxygen and sulfur or a combination of these atoms.

Said core-shell structured resin with rSPA in the inner core and hydrophilic outer layer can capture small Fc containing proteins such as half antibody (one heavy chain and one light chain), other small Fc fragments from antibody clipping from full antibody, or bispecific antibody or full Fc fusion protein. Those smaller fragments can enter the resin pore to interact with rSPA while full antibody or bispecific antibody or full Fc fusion protein is excluded from pores due the steric hinderance due to the restricted pore size. Said beads can be constructed by covalently conjugating rSPA or rSPAc via multiple or single point attachment.

By the similar approach, other affinity ligands or proteins such as native recombinant Protein G (J. Biol. Chem. 1991, 266, 399-405), Protein L (J. Immunol. 1988, 140, 1194-1197), Lectins, or others, as well as their mutants can be covalently conjugated to the inner core surface of porous resins while outer layer maintaining hydrophilic character as the size restrictor, such built resins will be useful in separation of larger proteins from smaller peptides or proteins containing Fc, Fab, sugar or other affinity ligand receptors.

Said chromatography medium can be advantageously applied to the separation of biomolecule mixtures with affinity and SEC mode. Outer layer SEC mode will exclude the biomolecules interreacting with inner layer functional groups of affinity tag, or IEX exchange groups. As an example, Resin 45 with hydrophilic character at outer layer and affinity dT25 ligand in the inner layer can be applied to separate the Poly A nucleotide tagged mRNA and LNP encapsulated mRNA.

Vaccine development has been a long-term endeavor against various diseases. Prophylactic vaccines can be used to prevent or ameliorate the effects of a future infection, while therapeutic vaccines are used to fight a disease that has already occurred, such as cancer. With the outbreak of Covid-19 in early 2020, vaccine development against Covid-19 becomes an urgent task in front of pharmaceutical industry as well as healthcare R&D organizations.

As known, LNP encapsulated mRNA, such as used in Covid-19 vaccine was prepared by assembly mRNA into positively charged LNP. Separation of free mRNA and encapsulated mRNA is a prerequisite to the delivery of final vaccine.

Resin 45 can be advantageously applied to the purification for this mRNA vaccine from its smaller production components such as free mRNA. Said Resin 45 is of affinity ligand of the inner core with will capture smaller impurity molecules, such as free mRNA, while large vaccine assembly will be collected in flow through due to outer layer SEC restriction. Such bead can be constructed with an affinity ligand at inner core, such as dT with a length ranging from 5 to 50, more preferred 10-40, most preferred 20-30. The length of said mRNA is of 30-4000 nt, preferable, 100-2000 nt.

Said hierarchical structured resin can also be appliable to solid supported materials, where chemical or biologic modifications can be done in different layers of said resin independently, which offers a new platform for developing new materials. For example, said resin can be applied to solid supported catalysts (SSC), including organic SSC, inorganic SSC, and enzymatic SSC. Here different catalytic systems can be modified into different layers of said resin with hierarchical structure, said catalytic systems can work independently for different substances through different kinds of transformation in a complexed mixture, or co-operate on a multiple-step synthesis in one shot based on division of chemical mechanism, such design can avoid deactivation of each catalyst during the transformations due to their cross-impaction.

Said resin design concept for SSC can be applied to solid support with hierarchical structure used for solid phase synthesis, such as solid phase peptide synthesis (SPPS), solid phase DNA synthesis (SPDS), solid phase organic synthesis (SPOS).

Said hierarchical construction methodologies, chemical kinetic control and masking-unmasking (protection-deprotection) processes as described in this invention, can be appliable to other support matrix modified with designed active functional group(s), such as allyl, vinyl groups, etc. Said matrix can be organic or inorganic materials, such as agarose, cellulose, dextran, chitosan, and their derivatives, silica and its derivative silica, glass, zirconium oxide, graphite, tantalum oxide etc.

It should be understood that the similar pore size between the core layer and the shell layer of the present medium is completely different from the pore structure of the existing medium which either has a small pore size in the core layer and a large pore size in the shell layer or has a large pore size in the core layer and a small pore size in the shell layer. The specific pore structure of the present medium is significantly important for the performance of the medium.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacture instructions. Unless indicated otherwise, parts and percentage are calculated by weight.

Unless otherwise defined, all professional and scientific terminology used in the text have the same meanings as known to the skilled in the art. In addition, any methods and materials similar or equal with the record content can apply to the methods of the invention. The method of the preferred embodiment described herein, and the material are only for demonstration purposes.

### EXAMPLES

### 1. Synthesis Examples of Polydispersed Porous Mother Beads

The properties of mother resins below, such as monomer and the ratio, particle size, porous structure, and alkene content, are listed in Table 2.

### Example 1. Synthesis of Polydispersed Porous EGDMA:AMA:GMA Mother Beads

Polyvinylpyrrolidone (PVP, 12.15 g, 40,000 g/mol) was dissolved into DI water (303.75 g) at rt to prepare aqueous phase mixture 1. Sodium dodecyl sulfate (SDS, 0.304 g), sodium sulfate (2.43 g), sodium nitrite (0.12 g) was dissolved into DI water (303.75 g) at rt to prepare aqueous phase mixture 2. Ethylene glycol dimethacrylate (EGDMA, 6.08 g), allyl methacrylate (AMA, 7.09 g), glycidyl methacrylate (GMA, 7.09 g), xylenes (6.08 g), n-hexane (6.08 g) and AIBN (0.41 g) were dissolved at rt to from oil phase mixture.

Charged aqueous phase mixture 1, aqueous phase mixture 2, and oil phase mixture into a round bottom flask (1 L) with N₂ purge. Stirred at 200 rpm using an overhead mechanical stirrer at rt, increased reaction temperature to 75° C. within 1 hour, and held the above temperature overnight for round 20 hours. Quenched the reaction temperature to below 30° C., washed the resulting resin with water 3 times, ethanol 3 times and water 3 times. The resulting Resin 1 was post-treated using conventional methods before collection: sonication, sieving, and sedimentation in DI water.

### Example 2. Synthesis of Polydispersed Porous EGDMA:AMA:GMA Mother Beads

Preparation procedure of Resin 1 was repeated, except a stirring speed of 150 rpm was used to make Resin 2.

### 2. Synthesis Examples of Monosized Porous Mother Beads (Examples 3-27)

The properties of mother resins below, such as monomer and the ratio, particle size, porous structure, and alkene content, are listed in Table 2.

### 2.1 Synthesis Examples of Monosized Low MW Polymer Seeds (Examples 3-9)

### Example 3. Synthesis of Primary Seed of PBMA (Seed 1)

PVP (4.0 g, 40,000 g/mol) was dissolved into DI water (100 g) to form Phase A mixture. Charged Phase A mixture, benzyl methacrylate (BMA, 30.0 g) and butyl 3-mercaptopropionate (0.93 g) into a 300 mL flask. Started stirring with N₂ purge for 10 minutes. Set the oil bath temperature to 75° C. and held the temperature for 1 hour. A pre-made solution of potassium persulfate (0.60 g) in DI water (20.0 g) was charged into the flask. Allowed polymerization continued overnight for around 20 hours. Quenched the reaction to below 30° C. After polymerization, aggregates were sieved and the resultant poly(benzyl methacrylate) (PBMA) seed solution (Seed 1) was collected.

### Example 4. Synthesis of Secondary Seed of PBMA (Seed 2)

Charged Seed 1 solution (3.33 g, dry weight basis), PVP solution (20.0 g, 40,000 g/mol, 4.0 wt %) into a 500 mL flask at rt. Started stirring with N₂ purge for 10 minutes to form Phase A mixture. Butyl 3-mercaptopropionate (3.56 g), Sodium Dodecylbenzene Sulfonate Surfactant (SDBS, 0.20 g), DI water (20.0 g) were mixed well in a beaker and then sonicated using a sonication horn for 10 minutes to form a Phase B mixture. Charged Phase B mixture into Phase A mixture slowly, started stirring at rt with N₂ protecting, and set up for 20 hours.

BMA (90.0 g), AIBN (1.20 g), SDBS (0.60 g), DI water (100 g) were mixed well in a beaker and then sonicated using a sonication horn for 10 minutes to form a Phase C mixture. Charged Phase C mixture into the flask slowly, started stirring at rt with N₂ protecting, and set up for 20 hours. Increased oil bath temperature to 70° C. and set up for 1 hour. Increased oil bath temperature to 80° C. and set up overnight for around 16 hours. Quenched the reaction to below 30° C. and seed solution (Seed 2) was collected. A particle size distribution analyzer (Better, Bettersize 2600E) was used to measure seed size and distribution. Summary of seed polymerization and seed properties are listed in Table 1.

### Example 5. Synthesis of Secondary Seed of PBMA/BA (Seed 3)

Preparation procedure of Seed 2 was repeated except Seed 1 solution (2.70 g, dry weight basis) was used to make Phase A mixture. BMA (45.0 g), BA (45.0 g), AIBN (1.20 g), SDBS (0.60 g), DI water (100 g) were used in Phase C mixture to make Seed 3 solution.

### Example 6. Synthesis of Tertiary Seed of PBMA/BA (Seed 4 and 5)

Preparation procedure of Seed 2 was repeated, and Seed 2 solution is used except BMA (45.0 g), BA (45.0 g), AIBN (1.20 g), SDBS (0.60 g), DI water (100 g) were used in Phase C mixture to make Seed 4 solution. Seed 4 is a monosized with a volume average D₅₀ value of 10.5 μm and D₉₀/D₁₀=1.22. The number average MW is 2,200 g/mol.

Preparation procedure of Seed 3 was repeated except Seed 3 solution dry weight basis (1.41 g) was used to make Phase A mixture. BMA (45.0 g), BA (45.0 g), AIBN (1.20 g), SDBS (0.60 g), DI water (100 g) were used in Phase C mixture to make Seed 5 solution.

### Example 7. Seed Synthesis from Primary Seed of PBA

### Synthesis of Primary Seed of PBA (Seed 6)

Sodium carbonate (0.4 g) was dissolved into DI water (85 g) to form Phase A mixture. Charged Phase A mixture into a 500 mL flask. Started stirring with N₂ purge for 10 minutes. Set the oil bath temperature to 80° C. Butyl acrylate (BA, 100.0 g), SDBS (0.50 g), sodium persulfate (0.06 g), DI water (82 g) were mixed well in a beaker and then sonicated using a sonication horn for 10 minutes to form a Phase B mixture. Charged Phase B mixture into the flask slowly within 4 hours at 80° C. Allowed polymerization continues for 60 minutes. Quenched the reaction to below 30° C. and seed solution (Seed 6) was collected.

### Synthesis of Secondary Seed of PBA (Seed 7)

Preparation procedure of Seed 2 was repeated, except Seed 6 solution (3.33 g, dry weight basis) was used to make Phase A mixture. BA (90.0 g), AIBN (1.20 g), SDBS (0.60 g), DI water (100 g) were used in Phase C mixture to make Seed 7 solution.

### Synthesis of Tertiary Seed of PBMA/BA (Seed 8)

Preparation procedure of Seed 2 was repeated, except Seed 7 solution (3.33 g, dry weight basis) was used to make Phase A mixture. BMA (45.0 g), BA (45.0 g), AIBN (1.20 g), SDBS (0.60 g), DI water (100 g) were used in Phase C mixture to make Seed 8 solution.

### Example 8. Seed Synthesis from Primary Seed of PBMA/BA

### Synthesis of Primary Seed of PBMA/BA (Seed 9)

Preparation procedure of Seed 1 was repeated, except BMA (27.0 g) and BA (3.0 g) were used make Seed 9 solution.

### Synthesis of Secondary Seed of PBMA/BA (Seed 10)

Preparation procedure of Seed 2 was repeated except Seed 9 solution (3.33 g, dry weight basis) was used to make Phase A mixture. BMA (45.0 g), BA (45.0 g), AIBN (1.20 g), SDBS (0.60 g), DI water (100 g) were used in Phase C mixture to make Seed 10 solution.

### Synthesis of Tertiary Seed of PBMA/BA (Seed 11)

Preparation procedure of Seed 2 was repeated, except Seed 10 solution (1.41 g, dry weight basis) was used to make Phase A mixture. BMA (45.0 g), BA (45.0 g), AIBN (1.20 g), SDBS (0.60 g), DI water (100 g) were used in Phase C mixture to make Seed 11 solution.

### Example 9. Seed Synthesis from Primary Seed of PST

### Synthesis of Primary Seed of PST (Seed 12)

Preparation procedure of Seed 1 was repeated, except styrene (30.0 g) were used to make Seed 12 solution.

### Synthesis of Secondary Seed of ST (Seed 13)

Preparation procedure of Seed 2 was repeated, except Seed 12 solution (8.5 g, dry weight basis) was used to make Phase A mixture. ST (90.0 g), AIBN (1.20 g), SDBS (0.60 g), DI water (100 g) were used in Phase C mixture to make Seed 13 solution.

### Synthesis of Secondary Seed of ST/BMA (Seed 14)

Preparation procedure of Seed 2 was repeated, except Seed 12 solution (5.9 g, dry weight basis) was used to make Phase A mixture. ST (45.0 g), BMA (45.0 g), AIBN (1.20 g), SDBS (0.60 g), DI water (100 g) were used in Phase C mixture to make Seed 14 solution.

### Synthesis of Tertiary Seed of ST/BMA (Seed 15)

Preparation procedure of Seed 2 was repeated, except Seed 14 solution (3.91 g, dry weight basis) was used to make Phase A mixture. ST (45.0 g), BMA (45.0 g), AIBN (1.20 g), SDBS (0.60 g), DI water (100 g) were used in Phase C mixture to make Seed 15 solution.

### Synthesis of Tertiary Seed of ST/BMA (Seed 16)

Preparation procedure of Seed 2 was repeated, except Seed 15 solution (5.14 g, dry weight basis) was used to make Phase A mixture. ST (45.0 g), BMA (45.0 g), AIBN (1.20 g), SDBS (0.60 g), DI water (100 g) were used in Phase C mixture to make Seed 16 solution.

### 2.2 Synthesis Examples of Monosized Porous Beads (Examples 10-27 and 78-90)

### Example 10. Synthesis of Monosized Porous EGDMA:AMA:GMA Mother Bead

Charged Seed 11 solution (2.268 g, dry weight basis), SDS solution (15.4 g, 1.0 wt %), carboxymethyl cellulose solution (50.0 g, 1.0 wt %), and DI water (146 g) into a 2 L round bottom flask at rt. Started stirring with N₂ purge for 10 minutes to form Phase A mixture. AIBN (2.0 g), SDS solution (249.0 g, 1.0 wt %), EGDMA (33.6 g), AMA (22.4 g), GMA (56.0 g), and dibutyl phthalate (208 g) were mixed in a beaker and then sonicated using a sonication horn for 10 minutes to from a Phase B mixture.

Charged Phase B mixture into Phase A mixture slowly, set temperature to 40° C. and set up for 4 hours. Added carboxymethyl cellulose solution (450.0 g, 1.0 wt %), mixed well, and increased temperature to 75° C. Held the reaction at the above temperature overnight for around 20 hours. Quenched the reaction temperature to below 30° C., washed the resulting resin with water 3 times, ethanol 3 times and water 3 times. The product, Resin 3, was post-treated using conventional methods if needed before collection: sonication, sieving, and sedimentation in DI water.

### Example 11. Synthesis of Monosized Porous EGDMA:AMA:GMA Mother Bead

Preparation procedure of Resin 3 was repeated, except EGDMA (33.6 g), AMA (39.2 g), and GMA (39.2 g) were used to make Resin 4.

### Example 12. Synthesis of Monosized Porous EGDMA:AMA:GMA Mother Bead

Preparation procedure of Resin 3 was repeated, except EGDMA (33.6 g), AMA (56.0 g), GMA (22.4 g) were used to make Resin 5.

### Example 13. Synthesis of Monosized Porous EGDMA:AMA:GMA Mother Bead

Charged Seed 11 solution (3.68 g, dry weight basis), SDS solution (15.4 g, 1.0 wt %), carboxymethyl cellulose solution (100.0 g, 1.0 wt %), and DI water (146 g) into a 2 L round bottom flask at rt. Started stirring with N₂ purge for 10 minutes to form Phase A mixture. AIBN (2.0 g), SDS solution (249.0 g, 1.0 wt %), EGDMA (33.6 g), AMA (39.2 g), GMA (39.2 g), dibutyl phthalate (22.4 g) and cyclohexanol (201.6 g) were mixed in a beaker and then sonicated using a sonication horn for 10 minutes to from a Phase B mixture.

Charged Phase B mixture into Phase A mixture slowly, set temperature to 40° C. and set up for 4 hours. Added carboxymethyl cellulose solution (450.0 g, 1.0 wt %), mixed well, and increased temperature to 75° C. Held the reaction at the above temperature overnight for around 20 hours. Quenched the reaction temperature to below 30° C., washed the resulting resin with water 3 times, ethanol 3 times and water 3 times. The product, Resin 6 was post-treated using conventional methods if needed before collection: sonication, sieving, and sedimentation in DI water.

### Example 14. Synthesis of Monosized Porous EGDMA:AMA:GMA Mother Bead

Preparation procedure of Resin 6 was repeated, except n-hexane (22.4 g) and xylenes (201.6 g) were used to make Resin 7.

### Example 15. Synthesis of Monosized Porous EGDMA:AMA:GMA Mother Bead

Preparation procedure of Resin 3 was repeated, except EGDMA (33.6 g), AMA (72.8 g), and GMA (5.6 g) were used to make Resin 8.

### Example 16. Synthesis of Monosized Porous EGDMA:AMA Mother Bead

Preparation procedure of Resin 3 was repeated, except EGDMA (33.6 g), and AMA (78.4 g) were used to make Resin 9.

### Example 17. Synthesis of Monosized Porous EGDMA:AMA Mother Bead

Preparation procedure of Resin 9 was repeated, except EGDMA (44.8 g), and AMA (67.2 g) were used to make Resin 10.

### Example 18. Synthesis of Monosized Porous EGDMA:AMA Mother Bead

Preparation procedure of Resin 9 was repeated, except EGDMA (22.4 g), and AMA (89.6 g) were used to make Resin 11.

### Example 19. Synthesis of Monosized Porous EGDMA:AMA:GMA Mother Bead

Charged Seed 5 solution (3.68 g, dry weight basis), SDS solution (15.4 g, 1.0 wt %), carboxymethyl cellulose solution (100.0 g, 1.0 wt %), and DI water (146 g) into a 2 L round bottom flask at rt. Started stirring with N₂ purge for 10 minutes to form Phase A mixture. AIBN (2.0 g), SDS solution (249.0 g, 1.0 wt %), EGDMA (33.6 g), AMA (56 g), GMA (22.4 g), dibutyl phthalate (89.6 g) and ethyl acetate (134.4 g) were mixed in a beaker and then sonicated using a sonication horn for 10 minutes to from a Phase B mixture.

Charged Phase B mixture into Phase A mixture slowly, set temperature to 40° C. and set up for 4 hours. Added carboxymethyl cellulose solution (450.0 g, 1.0 wt %), mixed well, and increased temperature to 75° C. Held the reaction at the above temperature overnight for around 20 hours. Quenched the reaction temperature to below 30° C., washed the resulting resin with water 3 times, ethanol 3 times and water 3 times. The product, Resin 12, was post-treated using conventional methods if needed before collection: sonication, sieving, and sedimentation in DI water.

### Example 20. Synthesis of Monosized Porous EGDMA:AMA:GMA Mother Bead

Preparation procedure of Resin 12 was repeated, except EGDMA (33.6 g), AMA (39.2 g), and GMA (39.2 g) were used to make Resin 13.

### Example 21. Synthesis of Monosized Porous EGDMA:AMA:GMA Mother Bead

Preparation procedure of Resin 13 was repeated, except dibutyl phthalate (128.8 g), and ethyl acetate (128.8 g) were used to make Resin 14.

### Example 22. Synthesis of Monosized Porous EGDMA:AMA:GMA Mother Bead

Preparation procedure of Resin 13 was repeated, except EGDMA (78.4 g), AMA (16.8 g), GMA (16.8 g), dibutyl phthalate (89.6 g) and ethyl acetate (134.4 g) were used to make Resin 15.

### Example 23. Synthesis of Monosized Porous DVB:AMA Mother Bead

Charged Seed 8 solution (2.18 g, dry weight basis), SDS (2.0 g), carboxymethyl cellulose solution (60.0 g, 1.6 wt %), and DI water (60 g) into a 2 L round bottom flask at rt. Started stirring with N₂ purge for 10 minutes to form Phase A mixture. AIBN (1.0 g), SDS solution (180 g, 0.5 wt %), DVB (56.0 g), AMA (14.0 g), xylenes (52.5 g), n-hexanol (52.5 g) and DI water (119 g) were mixed in a beaker and then sonicated using a sonication horn for 10 minutes to from a Phase B mixture.

Charged Phase B mixture into Phase A mixture slowly, set temperature to 40° C. and set up for 4 hours. Added carboxymethyl cellulose solution (202.64 g, 0.6 wt %), mixed well, and increased temperature to 75° C. Held the reaction at the above temperature overnight for around 20 hours. Quenched the reaction temperature to below 30° C., washed the resulting resin with water 3 times, ethanol 3 times and water 3 times. The product, Resin 16 was post-treated using conventional methods if needed before collection: sonication, sieving, and sedimentation in DI water.

### Example 24. Synthesis of Monosized Porous DVB:AMA Mother Bead

Preparation procedure of Resin 16 procedure was repeated, except DVB (45.5 g), and AMA (24.5 g) were used to make Resin 17.

### Example 25. Synthesis of Monosized Porous DVB:AMA Mother Bead

Preparation procedure of Resin 16 was repeated, except DVB (35.0 g), AMA (35.0 g) were used to make Resin 18.

### Example 26. Synthesis of Monosized Porous DVB:AMA:PVP Mother Bead

Charged Seed 15 solution (2.36 g, dry weight basis), SDS (2.71 g), carboxymethyl cellulose solution (81.36 g, 1.6 wt %), and DI water (81.36 g) into a 2 L round bottom flask at rt. Started stirring with N₂ purge for 10 minutes to form Phase A mixture. AIBN (1.36 g), SDS (1.22 g), DVB (52.21 g), AMA (9.49 g), PVP (33.22 g), toluene (90.17 g), n-hexanol (4.75 g), and DI water (404.2 g) were mixed in a beaker and then sonicated using a sonication horn for 10 minutes to from a Phase B mixture.

Charged Phase B mixture into Phase A mixture slowly, set temperature to 40° C. and set up for 4 hours. Added carboxymethyl cellulose solution (274.77 g, 0.6 wt %), mixed well, and increased temperature to 75° C. Held the reaction at the above temperature overnight for around 20 hours. Quenched the reaction temperature to below 30° C., washed the resulting resin with water 3 times, ethanol 3 times and water 3 times. The resulting Resin 19 was post-treated using conventional methods if needed before collection, sonication, sieving, and sedimentation in DI water.

### Example 27. Synthesis of Monosized Porous DVB:AMA:PVP Mother Bead

Preparation procedure of Resin 19 was repeated, DVB (33.22 g), AMA (28.48 g), and PVP (33.22 g) were used to make Resin 20.

### Example 78. Synthesis of Monosized Porous DVB:AMA Mother Bead

Charged Seed 5 solution (1.39 g, dry weight basis), SDS (2.0 g), carboxymethyl cellulose solution (60.0 g, 1.6 wt %), and DI water (60 g) into a 2 L round bottom flask at rt. Started stirring with N₂ purge for 10 minutes to form Phase A mixture. AIBN (1.0 g), SDS solution (180 g, 0.5 wt %), DVB (56.0 g), AMA (14.0 g), xylenes (52.5 g), n-hexanol (52.5 g) and DI water (119 g) were mixed in a beaker and then sonicated using a sonication horn for 10 minutes to from a Phase B mixture.

Charged Phase B mixture into Phase A mixture slowly, set temperature to 40° C. and set up for 4 hours. Added carboxymethyl cellulose solution (202.64 g, 0.6 wt %), mixed well, and increased temperature to 75° C. Held the reaction at the above temperature overnight for around 20 hours. Quenched the reaction temperature to below 30° C., washed the resulting resin with water 3 times, ethanol 3 times and water 3 times. The product, Resin 60 was post-treated using conventional methods if needed before collection: sonication, sieving, and sedimentation in DI water.

### Example 79. Synthesis of Monosized Porous DVB:AMA Mother Bead

Preparation procedure of Resin 60 procedure was repeated, except DVB (45.5 g), and AMA (24.5 g) were used to make Resin 61.

### Example 80. Synthesis of Monosized Porous DVB:AMA Mother Bead

Preparation procedure of Resin 60 was repeated, except DVB (35.0 g), AMA (35.0 g) were used to make Resin 62.

### Example 81. Synthesis of Monosized Porous EGDMA:AMA:GMA:DVB Mother Bead

Preparation procedure of Resin 3 was repeated, except 1.81 g (dry weight basis) of Seed 5 solution, EGDMA (33.6 g), AMA (33.6 g), GMA (33.6 g), DVB (11.2 g) and dibutyl phthalate (138.7 g) were used to make Resin 63.

### Example 82. Synthesis of Monosized Porous EGDMA:AMA Mother Bead

Preparation procedure of Resin 3 was repeated, except 1.48 g (dry weight basis) of Seed 5 solution, EGDMA (44.8 g), AMA (67.2 g) and dibutyl phthalate (138.7 g) were used to make Resin 64.

### Example 83. Synthesis of Monosized Porous DVB:AMA:GMA Mother Bead

Preparation procedure of Resin 60 was repeated, except 0.926 g (dry weight basis) of Seed 5 solution, DVB (3.5 g), AMA (3.5 g), GMA (63.0 g) were used to make Resin 65.

### Example 84. Synthesis of Monosized Porous DVB:AMA:GMA Mother Bead

Preparation procedure of Resin 65 was repeated, except DVB (3.5 g), AMA (63.0 g), GMA (3.5 g) were used to make Resin 66.

### Example 85. Synthesis of Monosized Porous DVB:AMA:GMA Mother Bead

Preparation procedure of Resin 65 was repeated, except DVB (63.0 g), AMA (3.5 g), GMA (3.5 g) were used to make Resin 67.

### Example 86. Synthesis of Monosized Porous EGDMA:DAM:GMA Mother Bead

Preparation procedure of Resin 3 was repeated, except 2.22 g (dry weight basis) of Seed 5 solution EGDMA (33.6 g), DAM (39.2 g), GMA (39.2 g) and dibutyl phthalate (138.7 g) were used to make Resin 68.

### Example 87. Synthesis of Monosized Porous EGDMA:AMA:DVB Mother Bead

Preparation procedure of Resin 3 was repeated, except 1.48 g (dry weight basis) of Seed 5 solution, EGDMA (11.2 g), AMA (67.2 g), DVB (33.6 g) were used to make Resin 69.

### Example 88. Synthesis of Monosized Porous EGDMA:AMA:DVB Mother Bead

Preparation procedure of Resin 69 was repeated, except EGDMA (33.6 g), AMA (67.2 g), DVB (11.2 g) were used to make Resin 70.

### Example 89. Synthesis of Monosized Porous EGDMA:AMA:DVB Mother Bead

Preparation procedure of Resin 69 was repeated, except EGDMA (22.4 g), AMA (67.2 g), DVB (22.4 g) were used to make Resin 71.

### Example 90. Synthesis of Monosized Porous EGDMA:AMA:GMA Mother Bead

Preparation procedure of Resin 3 was repeated, except 4.35 g (dry weight basis) of Seed 16 solution, EGDMA (56.0 g), AMA (28.0 g), GMA (28.0 g) and dibutyl phthalate (138.7 g) were used to make Resin 72.

### 3. Synthesis Examples of Monosized Core-Shell Porous Beads (Examples 28-61 and 91-101 in Table 3) General Method (GM): Chemical Kinetic Control Method

The synthesis examples showing herein below are for illustrative purpose only (FIG. 5 Route I, FIG. 6, and Table 3), and should not be constructed to limit the invention as defined by the appended claims. The methods are general and should be applied to the invented mother beads whether they are polydispersed or monosized.

GM1: Preparation of Medium Structurally Modified with OH-Lid Based Shell and Ligand Based Core.

### A. Hydrolysis of Epoxide Groups

Drained Resin 13 (50 g) was dissolved into dilute H₂SO₄ solution (150 mL). The mixture was magnetically stirred for overnight. Then resin GM1A was filtrated and washed with distilled water until pH neutral.

### B. Chemical Modification Method for OH-Lid Based Shell

Drained resin GM1A (44 g, the allyl content was determined by titration to be 1.2 mmol/g) and NaOAc (3.8 g) were dissolved into distilled water. An oversaturated Br₂ (2.32 g) water solution was added into a violently stirred solution. After that, the resin was washed with water, the drained gel was stirred with 2 M NaOH solution at 40° C. for overnight, followed by washing with water to give resin GM1B.

It should be noted that there are two exceptions (Resin 64 and Resin 72) at partial bromination step as following:

Drained resin GM1A from Resin 64 (20 g, the allyl content was determined by titration to be 2.5 mmol/g) and NaOAc (2.4 g) were dissolved into distilled water, then an oversaturated Br₂ (1.2 g) water solution was added into a violently stirred solution.

Drained resin GM1A from Resin 72 (20 g, the allyl content was determined by titration to be 1.1 mmol/g) and NaOAc (3.5 g) were dissolved into distilled water, then an oversaturated Br₂ (1.76 g) water solution was added into a violently stirred solution.

### C. Chemical Modification Method for Ligand Based Core

Water wet resin GM1B (90 g) and NaOAc (9 g) were dissolved into distilled water. Br₂ (6.7 g) was added into the flask with stirring for 1 h, and then sodium formate was added, and resin GM1C was washed with water and drained to dry. The drained resin GM1C was used for Core-Ligand coupling with various ligands showing below.

GM1a. Coupling of Ethylamine in the Core of the Beads

Drained resin GM1C (5 g) was stirred with EtNH₂ water solution for overnight, followed by washing with water and acetone.

GM1b. Coupling of Butylamine in the Core of the Beads

Drained resin GM1C (12 g) was stirred with BuNH₂ (15 mL) in water/DMF mixture for 10 h, followed by washing with water and acetone.

GM1c. Coupling of butylamine in the core of the beads

Drained resin GM1C (8 g) was stirred with 2 M NaOH/DMF mixture and BuNH₂ (4.5 mL) for overnight, followed by washing with water and acetone.

GM1d. Coupling of Butylamine in the Core of the Beads

Drained resin GM1C (8 g) was stirred with 2 M NaOH/DMF mixture and BuNH₂ (1 mL) for overnight, followed by washing with water and acetone.

GM1e. Coupling of Octylamine in the Core of the Beads

Drained resin GM1C (10 g) was stirred with water/DMF (30 mL) mixture and octylamine (10 mL) for overnight, followed by washing with water and acetone.

GM1f. Coupling of MBA in the Core of the Beads

Drained resin GM1C (10 g) was stirred with Me₂NBu (3 mL) in EtOH (30 mL) for overnight, followed by washing with water and acetone.

GM1 g. Coupling of TMA in the Core of the Beads

Drained resin GM1C (3 g) was stirred with Me₃N (45 wt %, 12 mL) in water at rt for overnight, followed by washing with water and acetone.

GM1h. Coupling of Iminodiacetic Acid (IDA) in the Core of the Beads

Drained resin GM1C (2.5 g) and iminodiacetic acid (2.5 g) were stirred in water/DMF mixture, the solution was adjusted to around pH 12.5 by 5 M NaOH, the mixture was stirred for overnight, followed by washing with water and acetone.

GM1i. Coupling of DMBA in the Core of the Beads

Drained resin GM1C (3 g) was stirred with DMBA (3 mL) in EtOH for overnight, followed by washing with water and acetone.

GM1j. Coupling of BMEA in the Core of the Beads

Drained resin GM1C (3 g) was stirred and BMEA (4 mL) in EtOH for overnight, followed by washing with water and acetone.

GM1k. Modification of the Core of the Beads with BMBA

Drained resin GM1C (3 g) and D, L-homocysteine (0.4 g) were stirred in 2 M KOH solution for 1 day. After washing with water and acetone, the drained gel was stirred with K₂CO₃ (2 g) in DMF (50 mL), then benzoyl chloride (2 mL) was added. The mixture was stirred at 25° C. for 12 h. After removing remaining K₂CO₃, the beads were washed with EtOH, water, and acetone, then stored in dryness.

GM1l. Coupling of Na₂SO₃ in the Core of the Beads

Drained resin GM1C (3 g) was stirred with saturated Na₂SO₃ solution at refluxing temperature, followed by washing with water and acetone.

GM1m. Coupling of 1-Hexanethiol in the Core of the Beads

Drained resin GM1C (2.5 g) and 1-hexanethiol (0.5 g) were stirred in water/DMF mixture, the solution was adjusted to around pH 12.5 by 5 M NaOH, the mixture was stirred for overnight, followed by washing with water and acetone.

GM1n. Coupling of 2-Phenylethanethiol in the Core of the Beads

Drained resin GM1C (3 g) and 2-Phenylethanethiol (0.5 g) were stirred with water/DMF mixture, the solution was adjusted to around pH 12.5 by 5 M NaOH, the mixture was stirred for overnight, followed by washing with water and acetone.

GM1o. Coupling of HS-C8-25 dT in the Core of the Beads

Drained resin GM1C (3 g) and HS-C8-25 dT (50 mg, customer synthesized) were stirred with water/DMF mixture, the solution was adjusted to around pH 8.5 by 2 M NaOH, the mixture was stirred for overnight. Then the beads were washed with water and stored with 20% EtOH in H₂O.

GM1p. Coupling of EDANS Dye in the Core of the Beads

Drained resin GM1C (3 g) and EDANS dye (200 mg) were stirred with water, the solution was adjusted to around pH 12.5 by 2 M NaOH, the mixture was stirred for overnight, followed by washing with water and acetone.

GM1q. Coupling of Congo Red Dye in the Core of the Beads

Drained resin GM1C (2 g) and Congo Red dye (300 mg) were stirred with a mixture of water and DMF, the solution was adjusted to around pH 12.5 by 5 M NaOH, the mixture was stirred for overnight, followed by washing with water and acetone.

GM1r. Coupling of 3-Aminophenylboronic Acid in the Core of the Beads

Drained resin GM1C (3 g) and 3-aminophenylboronic acid (0.5 g) were stirred with water/DMF mixture, the solution was adjusted to around pH 12.5 by 5 M NaOH, the mixture was stirred for overnight, followed by washing with water and acetone.

GM1s. Coupling of Protein a in the Core of the Beads

Drained resin GM1B (3 g) and mCPBA (0.5 g) were stirred with CH2C12 at rt for 6 h, then the intermediate resin was filtrated, washed with acetone, and collected in dryness. Said resin was stirred with a native recombinant Staphylococcal Protein A Ligand (300 mg, Repligen PN: 10-2001-XM) in a mixture of 60 mM NaHCO₃ for overnight. Then the beads were washed with water, then stored with 20% EtOH in H₂O.

GM1t. Coupling of EDANS in the Core of the Beads and Congo Red in the Intermediate Layer.

Drained resin GM1B (3 g) and EDANS dye (50 mg) were stirred with water, the solution was adjusted to around pH 12.5 by 2 M NaOH, the mixture was stirred for overnight, followed by washing with water and acetone to give GM1B1. Then, a partial bromination step was done as following, GM1B1 (3 g) was mixed with a NaOAc solution (0.26 g NaOAc in 15 mL H₂O), an oversaturated Br₂ (0.158 g) water solution was added into a violently stirred solution. After that, the resin was washed with water to give intermediate GM1B2. Drained resin GM1B2 (3 g) and Congo Red dye (300 mg) were stirred with a mixture of water and DMF, the solution was adjusted to around pH 12.5 by 5 M NaOH, the mixture was stirred for overnight, followed by washing with water and acetone to give Resin 73 using mother Resin 14.

GM2: Preparation of Medium Structurally Modified with NMe₃/SO₃H-Lid Based Shell and OH/Ligand Based Core.

### A. Hydrolysis of Epoxide Groups

Drained Resin 12 (25 g) was dissolved into dilute H₂SO₄ solution. The mixture was magnetically stirred for overnight. Then the resin GM2A was filtrated, and then washed with distilled water until pH neutral.

B. Chemical Modification Method for NMe₃/SO₃H-Lid Based Shell

Drained resin GM2A (10 g, the allyl content was determined by titration to be 2.2 mmol/g) and NaOAc (1.7 g) were dissolved into distilled water (80 mL). An oversaturated Br₂ (1.05 g) water solution was added into a violently stirred solution. After that, resin GM2B1 was washed with water, the drained resin GM2B1 was stirred with a mixture of water and Me₃N (45 wt %) at 60° C. for overnight. Then the beads were washed with 1 M HCl, 1 M NaOH, water, and acetone to give resin GM2B2. While said drained resin GM2B1 was stirred with saturated Na₂SO₃ at refluxing temperature for 1 day to produce resin GM2B3 after washing and drying processes.

### C. Chemical Modification Method for OH/Ligand Based Core

Resin GM2B2 (11 g) and NaOAc (3.8 g) were dissolved into distilled water (50 mL). Br₂ (3 g) was added into then flask with stirring for 1 h, and then sodium formate was added, and the resin GM2C1 was washed with water and drained to dry. Resin GM2C2 was produced through the same procedure as for GM2C1. Said resin GM2C1 and GM2C2 was used for core modifications showing below.

GM2a. Hydrolysis of Alkyl Bromide Group in the Core of the Beads

Resin GM2C1 (5 g) was stirred with 2 M NaOH solution at 40° C. for overnight, followed by washing with water and acetone.

GM2b. Coupling of 1-Hexanethiol in the Core of the Beads

Resin GM2C1 (3 g) and 1-hexanethiol (0.5 g) were stirred with water/DMF mixture, the solution was adjusted to around pH 12.5 by 5 M NaOH, the mixture was stirred for overnight, followed by washing with water and acetone.

GM2c. Hydrolysis of Alkyl Bromide Group in the Core of the Beads

Drained resin GM2C2 (4 g) was stirred with 2 M NaOH solution at 40° C. for overnight, followed by washing with water and acetone, then stored in dryness.

GM2d. Coupling of 1-Hexanethiol in the Core of the Beads

Drained resin GM2C2 (3 g) and 1-hexanethiol (0.5 g) were stirred with water/DMF mixture, the solution was adjusted to around pH 12.5 by 5 M NaOH, the mixture was stirred for overnight, followed by washing with water and acetone.

### 4. Synthesis Examples of Monosized Core-Shell Porous Beads (Examples 62-67 and 102 in Table 4). General Method: A Masking-Unmasking (Protection-Deprotection) Method

The synthesis examples showing herein below are for illustrative purpose only (FIG. 5 Route II, FIG. 8, and Table 4), and should not be constructed to limit the invention as defined by the appended claims. The methods are general and should be applied to the invented mother beads whether they are polydispersed or monosized.

### Example 62

Paraffin wax (2.5 g, CAS: 8002-74-2) was dissolved in ethyl acetate (100 mL) at 70° C. Resin 9 (10 g) was added to this solution. The mixture was heated at 70° C. for 5 min. The solvent was removed via rotary evaporator at 50° C. The solid (12.5 g) was dispersed in IPA/water (200 mL) containing NaOAc (8 g) and stirred with bromine (4 g) at rt for 5 min to produce bromide intermediate (54-I). The bromide intermediate (5 g) was stirred in a mixture of DMF and 2 M NaOH at 80° C. for 24 hr. The resin was collected by filtration and stirred in ethyl acetate (100 mL) at 70° C. twice, and then stirred with bromine (2 g) at rt for 10 min. The solid was collected by filtration to produce bromide intermediate (54-II, 4.8 g). This bromide was stirred with diethylamine (50% in water, 30 mL) at 40° C. for 16 hr. The solid was collected by filtration to yield Resin 54 (4.9 g).

### Example 63

The PMMA bromide intermediate (54-I, 5 g) was stirred with sodium sulfonate (5 g) in DMF/water at 50° C. for 16 hr. The resin was stirred in ethyl acetate (100 mL) at 70° C. twice to produce the sulfonate intermediate resin. The intermediate was stirred with bromine (2 g) at rt for 10 min. The solid was collected by filtration to produce bromide intermediate (55-I). This bromide was stirred with TMA (25% in water, 30 mL) at rt for 16 hr. The solid was collected by filtration to yield the Resin 55 (4.9 g).

### Example 64

Paraffin wax (1.5 g, CAS: 8002-74-2) was dissolved in ethyl acetate (200 mL) at 70° C. To this solution, Resin 8 (15 g) was added. The mixture was heated at 70° C. for 5 min. The solvent was removed via rotary evaporator at 50° C. to give resin (16.5 g), which was dispersed in a mixture solvent IPA and water (150 mL) containing NaOAc (4 g). The bromine (3.5 g) was added. The mixture was stirred at rt for 5 min to produce bromide intermediate (56-I, 19 g) after clean process. The intermediate was treated with 10% DMF in 2 M NaOH at 55° C. for 48 hr. The solid was collected by filtration and further treated with ethyl acetate (80 mL) at 70° C. twice to give the resin (56-II), which was stirred with bromine (2 g) at rt for 10 min. The solid was collected by filtration to produce bromide intermediate (56-III), which was stirred with trimethyl ammonia (25% in water) for 16 hr to produce Resin 56 (15.7 g).

### Example 65

Paraffin wax (1.5 g: CAS: 8002-74-2) was dissolved in ethyl acetate (200 mL) at 70° C., then Resin 2 (12 g) was added. The mixture was heated at 70° C. for 5 min. The solvent was removed via rotary evaporator at 50° C. to give resin (13.5 g), which was dispersed in a mixture solvent IPA and water (150 mL) containing NaOAc (4 wt %). Then bromine (3.5 g) was added. The mixture was stirred at rt for 5 min to produce the bromide intermediate (57-I, 14.8 g), which was treated with 10% DMF in 2 M NaOH at 55° C. for 48 hr. The solid was collected by filtration and further treated with ethyl acetate (80 mL) at 70° C. twice. Further treatment of said resin with bromine (3 g) at rt for 10 min produced bromide intermediate (57-II), which was further stirred with 10% sodium sulfite solution at 50° C. for 16 hr to produce Resin 57 (13.2 g).

### Example 66

Following the procedure of Example 64, Resin 16 (10 g) was used instead of Resin 8 to produce Resin 58 (10.3 g).

### Example 67

Following the procedure of Example 65, Resin 19 (10 g) was used instead of Resin 2 to produce Resin 59 (10.4 g).

### Example 102

Following the procedure of Example 62, Resin 61 (10 g, 53.8 μm bead size) was used and bromide intermediate (84-I) and bromide intermediate (84-II) were generated. Bromide intermediate 84-II (2 g) and EDANS dye (30 mg) were stirred with water, the solution was adjusted to around pH 12.5 by 2 M NaOH, the mixture was stirred for overnight, followed by washing with water and acetone to give Resin 84. The core-shell two-layer structure was clearly visualized through confocal laser scanning microscopy (CLSM, LSM 880) studies, as shown in FIG. 10B.

### 5. Comparative Resin Examples

Comparative Resin 1 (Capto Core 700 resin from GE Healthcare) Polydispersed core-shell commercial agarose resin (˜85 μm) with a Dextran modified shell and a core modified with octylamine. Capto Core 700 is a polydispersed porous resin with D₅₀ value of 88.3 μm and D₉₀/D₁₀=2.22, as shown in FIG. 2D. Its property is listed in Table 2 and 3.

Comparative Resin 2 (Oasis HLB Resin from Waters)

Polydispersed DVB-PVP commercial resin (˜30 μm) with conventional porous structure. Oasis HLB resin is a polydispersed porous resin with D₅₀ value of 26.8 μm and D₉₀/D₁₀=1.86, as shown in FIG. 2E. Its property is listed in Table 2.

Comparative Resin 3 (Generik MC Resin with Epoxide Functional Groups from Sepax Technologies Inc.)

Polydispersed EGDMA-GMA commercial resin (˜60 μm) with conventional porous structure. Generik MC is a polydispersed porous resin with D₅₀ value of 59.4 μm and D₉₀/D₁₀-1.99. Its property is listed in Table 2.

### Comparative Resin 4 (Waters Oasis MAX Resin).

Polydispersed DVB-PVP commercial resin (˜30 μm) with conventional porous structure and MBA modified ligand. According to the Waters' brochure, Oasis MAX resin was made from Oasis HLB resin. Its property is listed in Table 3.

### 6. General Characterization and Evaluation Methods

Resin (and seed) characterization. Particle size and particle size distribution was measured by a Beckman Coulter Particle Size Analyzer (Beckman) or Light Scattering particle size distribution analyzer (Better, Bettersize 2600E). The volume average particle diameter (D₅₀) and particle size distribution (D₉₀/D₁₀) were reported. Light microscopy, scanning electron microscope (SEM) and fluorescent confocal microscopy (CLSM, LSM 880) were also used in a comprehensive way to assess bead size (and distribution).

Seed MW analysis. MW of seeds was determined through a PS-DVB SEC column (Mono GPC, 5 μm, 300 Å, 7.8×300 mm stainless steel column, PN: 230300-7830, Sepax Technologies, Inc.) at 1.0 mL/min in THE at rt. The SEC column was calibrated using a set of polystyrene (PS) GPC MW standards (Agilent, PN: PL2010-0104). The number average MW was reported with respect to PS MW.

Functional groups. The content of allyl group of mother resins (in Table 2) was determined through allyl titration, as well as bromine atom elemental analysis in some cases. The changes of allyl group intensity (e.g., Resin 5) against amounts of bromine used in partial bromination step were monitored qualitatively by FT-IR studies, as shown in FIG. 7, and further confirmed quantitatively through bromine elemental analysis. Here the allyl content of the Resin 5 from bromine elemental analysis (0.912 mmol/mL) was consistent with the result from allyl titration (0.96 mmol/mL). Meanwhile, completion of bromohydrin hydrolysis was confirmed by bromine elemental analysis as well, which was under, 4000 ppm, the detection limit.

Bead morphology. Light microscopy, SEM, and fluorescent confocal microscopy (CLSM, LSM 880) were used in a comprehensive way to characterize the synthesized beads. Most beads for SEM experiments are gently placed on a conductive carbon tape while Resin 29 is partially broken by pressing with a spatula in a control manner in order to expose interior porous structure (cross-sectioned area). One SEM (HITACHI cold field SEM S-4800) is in Suzhou Institute of Nano-Tech and Nano-Bionics (SINANO), Chinese Academy of Sciences, and SEM samples were sputter coated with Au before SEM observation. Another SEM (HITACHI cold field SEM S-4700) is in Bio-Imaging Center at the University of Delaware, and SEM samples are sputter coated with Pt before SEM observation.

Porous structure characterization. SEM, fluorescent confocal microscopy, a specific surface area and porosity analyzer (Micromeritics TriStar II Plus), a mercury intrusion analyzer (Micromeritics MicroActive AutoPore) were used in a combinational way to characterize the synthesized beads.

Hierarchical layer structure (core-shell case). Visualization of core-shell two-layer hierarchical structure was achieved through CLSM (LSM 880) studies, which was conducted with Resin 46 and Resin 84 labeled with EDANS, and Resin 47 labeled with Congo red dye in their cores. Here intermediate resin, GM1C, with hydroxylated shell and brominated core, was chosen for core-dye modification, as shown in FIG. 10A and FIG. 11A. In order to illustrate the efficiency of masking-unmasking way core-shell construction, Resin 84 with EDANS in its core and a hydroxylated shell were design, synthesized, and visualized, as shown in FIG. 10B. Meanwhile, a resin with three layers, Resin 73, was also designed, prepared and visualized. The core-shell three-layer structure was clearly visualized through confocal laser scanning microscopy (CLSM, LSM 880) studies, as shown in FIG. 11B. Following through procedure GM1t the outer layer was modified with EDANS through sequential partial bromination/amination process, and Congo red dye modification in the middle layer was achieved through repeating this process, and the core was modified with hydroxy.

### 7. Application Examples of Core-Shell Porous Resin (Examples 68-77)

### Example 68. NaNO₂ Retention Time Test for Resin 23 and Oasis-MAX Resin

For comparison, Resin 23 and Oasis-MAX resin were packed in 2.1×50 mm stainless steel columns. 2 μL of 3.5 mg/mL NaNO₂ sample was injected, testing at a flow rate of 0.3 mL/min under the conditions, as shown in Table A. The retention times of NaNO₂ from Resin 23 and Oasis-MAX columns were evaluated to be 7.1 mins and 6.5 mins, respectively, this result was further confirmed quantitatively through IEX capacity studies, which was determined to be 1.03 meq/g and 0.319 meq/g, respectively, as shown in FIG. 12B. The longer retention times of NaNO₂ from Resin 23 indicated its higher functional group density compared to that of Oasis-MAX resin.

TABLE A
NaNO₂ test conditions

| | |
|---|---|
| Instrument | Agilent HPLC 1260 |
| Column | Resin 23 (2.1 × 50 mm) |
| | Oasis-MAX (2.1 × 50 mm) |
| Mobile phase | A: 20 mM TRIS, pH 7 |
| | B: 20 mM TRIS + |
| | 0.5M NaCl |
| Flow rate | 0.30 mL/min |
| Gradient | 0-10% B in 10 min |
| Temperature | Ambient |
| Detection | UV (214 nm) |
| Sample | NaNO₂ (3.5 mg/mL) |
| Injection volume | 2 μL |

### Example 69. Erbitux NSB Studies of Resin 23 and Oasis-MAX Resin

The NSB studies (no-specific binding) were done with 2.1×50 mm columns packed with Resin 23 and Oasis-MAX resin. Multiple Erbitux runs (each injection: 2 μL of 1.0 mg/mL Erbitux) were done through columns in 5 mins at a flow rate of 0.30 mL/min, as shown in Table B. The sample peak areas were recorded at a UV detector signal at 280 nm. For Resin 23 column, 10 runs were done by using buffer A, all Erbitux samples were almost fully recovered, while all Erbitux over 10 runs stuck in the column packed with Oasis-MAX resin even by using buffer B (FIG. 13).

TABLE B
Erbitux NSB test conditions

| | |
|---|---|
| Instrument | Agilent HPLC 1260 |
| Column | Resin 23 (2.1 × 50 mm) |
| Mobile phase | A: 150 mM sodium phosphate, pH 7 |
| | B: 150 mM sodium phosphate + |
| | 150 mM NaCl, pH 7 |
| Flow rate | 0.30 mL/min |
| Gradient | 100% A or 100% B in 5 min |
| Temperature | Ambient |
| Detection | UV (280 nm) |
| Sample | Erbitux (1.0 mg/mL) |
| Injection volume | 2 μL |

### Example 70. Tween 80 Quantification Studies of Resin 23 Column

Column (2.1×50 mm) packed with Resin 23 was used for Tween quantification method development. 5 μL of each water solution of Tween 80 with different concentrations (0.003%, 0.006%, 0.013%, 0.025%, 0.050%, 0.100%, w/w) were injected, and tested under the conditions shown in Table C. The sample peak areas eluting at 9.75 mins were recorded through Evaporative Light Scattering Detector (ELSD). The peak areas of each standard solution against Tween 80 concentrations (0.003%, 0.006%, 0.013%, 0.025%, 0.050%, 0.100%, w/w) were plotted by linear regression after logarithmic processing by using the following formula:

log I=b*log m+log k
where I is the intensity of light, m is the mass of the scattering particles, m, k and b are constants, log I vs log m provides a linear response. The linear equation for said Resin 23 column was y=0.8043x+5.7371, with correlation coefficient R²>0.95 in our first try. The results for linearity showed that Resin 23 column with the present method had excellent linearity in the range of 0.003% to 0.1% (w/w) of Tween 80.

TABLE C
Tween quantification test conditions

| | |
|---|---|
| Instrument | Agilent HPLC 1260 |
| Column | Resin 23 (2.1 × 50 mm) |
| Mobile phase | A: 2% formic acid in H₂O |
| | B: 2% formic acid in IPA |
| Flow rate | 1.00 mL/min |
| Gradient | 10-20% B (0.00-2.50 min) |
| | 20% B (2.50-8.50 min) |
| | 20-100% B (8.50-8.75 min) |
| | 100% B (8.75-11.25 min) |
| Temperature | Ambient |
| Detection | ELSD |
| Sample | Tween 80/H₂O (w/w): |
| | 0.003%, 0.006%, 0.013%, |
| | 0.025%, 0.050%, 0.100% |
| Injection volume | 5 μL |

### Example 71. Tween Trap Capacity Studies of Resin 23 Column

The columns (2.1×50 mm) packed with Resin 23 and Oasis-MAX medium were used for Tween trap capacity tests. Multiple runs of Tween 80 sample (each injection: 50 μL of 0.1% Tween 80, w/w) were done under the conditions in Table D through Resin 23 column. The flow through peak areas of each run were recorded through ELSD. The breakthrough of Tween 80 was observed to be 0.65 μg, which showed the capacity at the breakthrough point was 16 times higher than that of Oasis-MAX column (0.04 μg) under the same conditions, as shown in FIG. 14.

TABLE D
Tween trap test conditions

| | |
|---|---|
| Instrument | Agilent HPLC 1260 |
| Column | Resin 23 (2.1 × 50 mm) |
| | Oasis-MAX (2.1 × 50 mm) |
| Mobile phase | 2% formic acid in H₂O |
| Flow rate | 1.00 mL/min |
| Gradient | 100% A in 2 mins |
| Temperature | Ambient |
| Detection | Agilent ELSD G4218A |
| Sample | 0.100% Tween 80 |
| Injection volume | 50 μL (Resin 23) |
| | 10 μL (Waters Oasis-MAX) |

### Example 72. Separation of Tween/Protein Under Non-Denaturing Conditions

Resin 23 was packed into a 2.1×50 mm column. 1 μL of a sample mixed with Erbitux (0.4 mg/mL) and Tween 80 (0.08%, wt %) was analyzed on said column with ELSD as detector. Erbitux was excluded from pores, eluted, and collected naturally at 0.25 min with 50 mM ammonium acetate, while Tween 80 was trapped in the resin pore with more hydrophobic butylamine, and only eluted at 9.8 min with 100% isopropanol (IPA), as shown in FIG. 15. When the same conditions were applied to Oasis-MAX resin, most of Erbitux was stuck on the column (data is not shown).

TABLE E
Tween/protein separation test conditions

| | |
|---|---|
| Instrument | Agilent HPLC 1260 |
| Column | Resin 23 (2.1 × 50 mm) |
| Mobile phase | A: 50 mM ammonium acetate, pH 7.0 |
| | B: IPA |
| Flow rate | 1.0 mL/min |
| Gradient | 0% B (0.00-8.74 min) |
| | 0-100% B (8.74-8.75 min) |
| | 100% B (8.75-11.25 min) |
| Temperature | Ambient |
| Detection | ELSD |
| Sample | water, |
| | 0.08% Tween 80 in H₂O (w/w), |
| | 0.4 mg/mL Erbitux, |
| | 0.4 mg/mL Erbitux + 0.08% Tween 80 |
| Injection volume | 1 μL |

### Example 73. VLP Purification Application

Several polypropylene columns (7.3×100 mm) were packed with Resin 28-32. Only the column packed with Resin 29 for preparative chromatogram was shown here for illustrative purpose. A mixture of VLP and impurities was injected and pumped through said column, testing flow rate of 83 cm/h by using a buffer shown in Table F. The sample peak eluting at 30 mins was recorded through UV at 280 nm. Desired VLP was collected in the flow through mode, while smaller molecules such as DNA fragments and endotoxin were bound to the inner pore surface with weak anion exchange functional group, butylamine, which can be eluted at the elevated NaCl concentration. A CIP with 1 M NaOH sanitized the column and restored the column for next round purifications, as shown in FIG. 16.

TABLE F
VLP separation test conditions

| | |
|---|---|
| Instrument | Generik SCG 30 |
| Column | Resin 29 (7.3 × 100 mm) |
| Mobile phase | 50 mM Tris + 0.5M NaCl, pH 7.5 |
| Flow rate | 83 cm/h |
| CIP | 1M NaOH in 30% IPA aqueous solution |
| Temperature | Ambient |
| Detection | UV at 280 nm |
| Sample | VLP, 80-90 nm in size |
| Injection volume | 1 CV |

### Example 74. Preparative Purification of a Crude mAb Sample

Resin 49 was packed into a 11×270 mm column. A mAb fermentation broth (200 ml, 7.8 CV, 3.2 mg/mL) was injected and pumped through said column, testing flow rate of 4.27 mL/min by using a buffer for equilibrium and loading shown in Table G. The sample peak area eluting at 23 mins was recorded through UV at 280 nm by using elution buffer after two additional washing steps to remove impurities, as shown in FIG. 18. Desired antibody was recovered in 95% yield with 97% purity. The column can be sanitized with a CIP with 0.1 M NaOH for 20 min, and then can be reused through re-equilibrium conditions (20 mM Na-phosphate, 150 mM NaCl, pH 7.4).

TABLE G
mAb purification conditions

| | |
|---|---|
| Instrument | ÄKTA pure 25 |
| Column | Resin 49 (11 × 270 mm) |
| Equilibrium and | 20 mM Na-phosphate + 50 mM |
| loading | NaCl, pH 7.4 |
| Elution | 100 mM Gly-HCl, pH 3.5 |
| Flow rate | 4.27 mL/min |
| CIP | 0.1M NaOH, 20 min |
| Detection | UV at 280 nm |
| Sample | 200 ml antibody fermentation |
| | broth (7.8 CV, 3.2 mg/mL) |
| Loading capacity | 25.0 mg/mL |

### Example 75. Protein A Separation Application (Prophetic Example)

A mixture of bispecific mAb XY and corresponding half-body X and half body Y is injected to a 2.1×50 mm column packed from Resin 49. The bispecific mAb XY is eluted early with the mobile phase (50 mM phosphate buffer containing 500 mM NaCl). While the half bodies are eluted with 100 mM Glycine, pH 2.5, suggesting these half-bodies are bound to rSPA inside the pores until the low pH mobile phase disrupts the rSPA and Fc interaction.

### Example 76. Preparative Purification of a Crude mRNA Sample

Resin 45 with dT25 ligand was packed into a 7.8×300 mm column. A crude mRNA sample (˜1000 nt, made from IVT upstream process) was injected and pumped through said column, testing flow rate of 0.5 mL/min by using a buffer for equilibrium and loading shown in Table H. The sample peak area eluting at 21 mins was recorded through UV at 260 nm. The overall recovery yield of mRNA was 63%, which can be improved in scale-up process under optimized conditions. Here mRNA was purified through affinity binding mechanism between polyA tail of said mRNA, and dT25 ligand in Resin 45.

TABLE H
mRNA separation conditions

| | |
|---|---|
| Instrument | ÄKTA pure 25 |
| Column | Resin 45 (11 × 270 mm) |
| Equilibrium, loading, | 10 mM Tris pH 7.4 |
| washing buffer | |
| Elution | water |
| Flow rate | 0.5 mL/min |
| CIP | 0.1M NaOH |
| Detection | UV at 260 nm |
| Sample | 300 uL mRNA IVT broth, 5.5 |
| | mg mRNA/mL diluted with |
| | equilibrium buffer |
| Loading capacity | 1.5 mg/mL |
| Sample loading | 10 CV |

### Example 77. mRNA Encapsulation Yield Study (Prophetic Example)

A column (2.1×50 mm) is packed with Resin 45. A mixture of mRNA and LNP encapsulated mRNA is injected and pumped through said column. LNP encapsulated mRNA is excluded from said column even though the LNP carries the positive charge, which would interact with the negatively charged dT tag otherwise, while free mRNA with PolyA tag will be captured with dT25 inside pore surface. mRNA encapsulation yield is calculated as a ratio of mRNA encapsulated in LNP to the total of mRNA encapsulated and free mRNA.

Example 103. Bacteriophage purification application. Application of purification and separation of a crude bacteriophage (about 80 nm in size, isoelectric point <7)

A stainless steel chromatography column packed with Monomix Core 60 (part number: 290160990, further developed based on Resin 31 in the present invention) from SEPAX TECHNOLOGIES, INC. was used in this application example.

The purification process was shown in FIG. 20:
Equilibration: rinse and equilibrate the chromatography column with a equilibration buffer solution (20 mM phosphate salt buffer solution, pH 6.0) at a flow rate of 83 cm/h;
Sample loading: load 1 column volume of bacteriophage sample at a flow rate of 83 cm/h;
Post-equilibration: rinse the chromatography column with a buffer solution (20 mM phosphate salt buffer solution, pH 6.0) and collect the flow-through sample using 280 nm UV light;
CIP: rinse the column with 3 CV of 1.0 M NaOH aqueous solution;
Storage: rinse the column with 3 CV of purified water, then 3 CV of 20% ethanol aqueous solution, and store said column at room temperature in 20% ethanol.

The experimental results showed that the chromatography column packed with Monomix Core 60 chromatography medium has the following characteristics:
1) The required bacteriophage can be collected in the flow-through mode, and the smaller molecular weight impurities are bound on the surface of the core pore by a weak anion exchange functional group (amino group), and then eluted under a CIP condition to achieve the goal of separation and purification of bacteriophage.
2) Using the chromatography medium, the recovery rate of bacteriophage is >90%, and its purity is >84%.
3) Using an aqueous solution of 1.0 M NaOH as the CIP solution, the chromatography column can be disinfected and regenerated for the next round of purification. The CIP cleaning conditions is not requested to use organic solvents such as isopropanol, which is convenient for customers to use, and is superior to the 1.0 M NaOH 30% isopropanol aqueous solution used in Capto™ Core 700 CIP cleaning condition as reported in the literature.

TABLE I
bacteriophase separation conditions

| | |
|---|---|
| Instrument | Generik SCG 30 |
| Column | Monomix Core 60 (7.3 × 100 mm) |
| Mobile phase | 20 mM phosphate salt buffer, pH 6.0 |
| Flow rate | 83 cm/h |
| CIP | 1M NaOH aqueous solution |
| Temperature | Ambient |
| Detection | UV at 280 nm |
| Sample | bacteriophase, 80-90 nm in size, isoelectric |
| | point < 7 |
| Injection volume | 1 CV |

### Example 104. Adeno Virus Purification Application

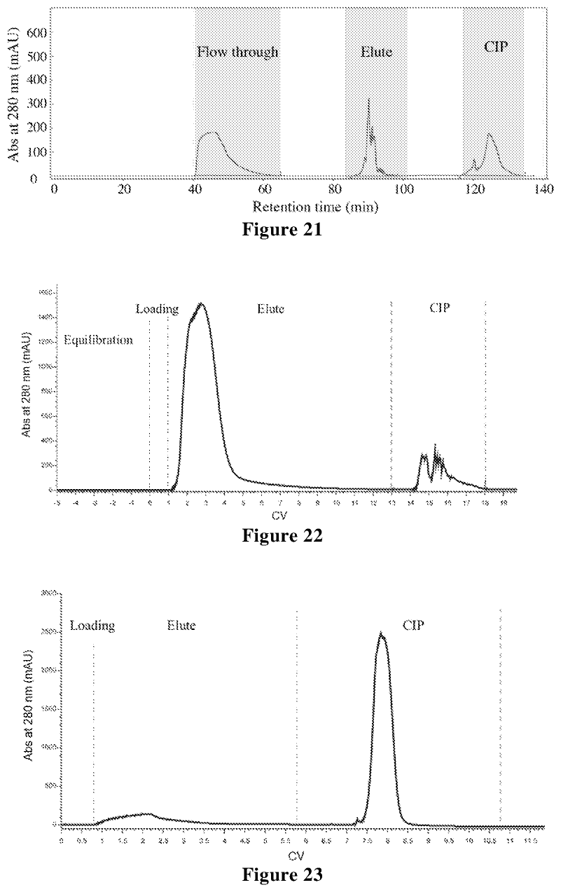

Process chromatogram of an Adeno virus sample in the polishing step using a polypropylene column packed with Resin 31. A mixture of Adeno virus sample was injected and pumped through said column, testing flow rate of 90 cm/h by using a buffer shown in Table J. The sample peak eluting at 47 mins was recorded through UV at 280 nm. Adeno virus was collected in the flow through mode, while smaller molecules such as nuclease, DNA fragments and host cell proteins (HCP) were bound to the inner pore surface with weak anion exchange functional group, butylamine, which can be eluted with a NaOH aqueous solution. A CIP with 1.0 M NaOH aqueous sanitized the column and restored the column for next round purifications, as shown in FIG. 21. Purification results show viral particles-based recovery of 92%, nuclease <0.1 ng/mL, DNA level of 0.12 ng/mL, and HCP of 2.1 ng/mL.

The same Adeno virus was purified using a column packed with Cytiva's Capto™ Core 700 resin. Similar purification results were achieved (viral particles-based recovery of 93%, nuclease <0.1 ng/mL, DNA level of 0.07 ng/mL, and HCP of 2.5 ng/mL). But this column request 1.0 M NaOH in 30% IPA aqueous to do CIP (chromatogram not shown).

TABLE J
Adeno virus purification conditions

| | |
|---|---|
| Instrument | Generik SCG 30 |
| Column | Resin 31 (7.3 × 100 mm) |
| Equilibration | 550 mM NaCl, 50 mM HEPES, pH 7.4 |
| buffer | |
| Flow rate | 90 cm/h |
| CIP | 1.0M NaOH aqueous solution |
| Temperature | ambient |
| Detection | UV at 280 nm |
| Sample | Adeno virus (~5.0 × 10\hat{ }11 VP/mL, |
| | 80-100 nm in size |
| Loading volume | 1.5 CV |

### Example 105. Inactivated Flu Vaccine Purification Application

Process chromatogram of a crude inactivated flu vaccine sample in the polishing step using a 1.0 mL prepacked column with Resin 29. A crude inactivated flu vaccine sample was injected and pumped through said column, testing flow rate of 63 cm/h by using a buffer shown in Table K. The sample peak eluting at 2.8 CV was recorded through UV at 280 nm. Inactivated flu vaccine was collected in the flow through mode, while smaller molecule impurities were bound to the inner pore surface with weak anion exchange functional group, butylamine. A CIP with 0.5 M NaOH in 30% IPA aqueous sanitized the column and restored the column for next round purifications, as shown in FIG. 22.

TABLE K
Inactivated flu vaccine purification conditions

| | |
|---|---|
| Instrument | ÄKTA pure 25 |
| Column | 1.0 mL prepacked column with Resin 29 |
| Equilibration | 10 mM PB + 0.15M NaCl, pH 7.1 |
| buffer | |
| Flow rate | 63 cm/h |
| CIP | 0.5M NaOH in 30% IPA aqueous |
| Temperature | ambient |
| Detection | UV at 280 nm |
| Sample | inactivated flu vaccine |
| Loading volume | 1.0 CV |

### Example 106. Plasmid Purification Application

Process chromatogram of a crude a plasmid sample in the capture step using a glass column packed with Resin 29. A crude plasmid sample was pretreated by dialysis and then injected and pumped through said column, testing flow rate of 150 cm/h by using a buffer shown in Table L. The sample peak eluting at 2.0 CV was recorded through UV at 260 nm. Supercoil plasmid was collected in the elution, while smaller molecule impurities and open plasmid were tightly bound to the inner pore surface with weak anion exchange functional group, butylamine. A CIP with 1.0 M NaOH aqueous sanitized the column and restored the column for next round purifications, as shown in FIG. 23.

TABLE L
Plasmid purification conditions

| | |
|---|---|
| Instrument | Generik SCG 30 |
| Column | 6.6 × 100 mm glass column packed with |
| | Resin 29 |
| Loading buffer | 50 mM Tris, 10 mM EDTA, pH 7.1, 1.5 CV |
| Elution buffer | 50 mM Tris, 10 mM EDTA, pH 7.1, 5 CV |
| CIP | 1.0M NaOH aqueous, 5 CV |
| Flow rate | 150 cm/h |
| Temperature | ambient |
| Detection | UV at 260 nm |
| Sample | Plasmid (8 kbp, sc and open isoform) |
| Loading volume | 0.8 CV |

TABLE 1
Summary of seed polymerization and seed properties.

| | | 1st Seed | 2nd Seed | 3rd Seed | 4th Seed | D₅₀ | D₉₀/D₁₀ | Mn |
|---|---|---|---|---|---|---|---|---|
| Example | Seed | monomer(s) | monomer(s) | monomer(s) | monomer(s) | (μm) | (a) | (g/mol)(b) |
| 3 | Seed 1 | BMA | | | | 1.17 | 1.10 | 12,000 |
| 4 | Seed 2 | BMA | BMA | | | 3.5 | 1.16 | 4,500 |
| 5 | Seed 3 | BMA | BMA/BA | | | 3.8 | 1.18 | 5,100 |
| 6 | Seed 4 | BMA | BMA | BMA/BA | | 10.5 | 1.22 | 3,200 |
| 6 | Seed 5 | BMA | BMA/BA | BMA/BA | | 14.9 | 1.25 | 2,900 |
| 7 | Seed 6 | BA | | | | 0.39 | 1.03 | 62,000 |
| 7 | Seed 7 | BA | BA | | | 1.16 | 1.08 | 4,100 |
| 7 | Seed 8 | BA | BA | BMA/BA | | 3.5 | 1.12 | 3,300 |
| 8 | Seed 9 | BMA/BA | | | | 0.60 | 1.03 | 10,500 |
| 8 | Seed 10 | BMA/BA | BMA/BA | | | 1.76 | 1.05 | 4,500 |
| 8 | Seed 11 | BMA/BA | BMA/BA | BMA/BA | | 7.0 | 1.07 | 2,700 |
| 9 | Seed 12 | ST | | | | 1.42 | 1.05 | 15,000 |
| 9 | Seed 13 | ST | ST | | | 3.05 | 1.35 | 4,300 |
| 9 | Seed 14 | ST | ST/BMA | | | 3.5 | 1.07 | 4,600 |
| 9 | Seed 15 | ST | ST/BMA | ST/BMA | | 9.9 | 1.21 | 2,600 |
| 9 | Seed 16 | ST | ST/BMA | ST/BMA | ST/BMA | 25.2 | 1.25 | 2,100 |

(a) Particle size analysis was performed using a light scattering method.
(b) The number average MW was reported with respect to PS MW standards

TABLE 2
Summary of polymerizations and mother bead properties.

| | | | | | | Porous structure (a) | | | |
|---|---|---|---|---|---|---|---|---|---|
| | | | Seed | | | | | Average | Alkene |
| | | | in | Particle size | | Surface | Pore | pore | content |
| | | Monomer and its | Table | D₅₀ | D₉₀/ | area | volume | size | (mmol/g) |
| Example | Resin | (wt %) ratio | 1 | (μm) | D₁₀ | (m²/g) | (mL/g) | (Å) | (b) |
| 1 | 1 | E(30):A(35):G(35) | NA | 35.1 | 1.91 | 85 | 0.42 | 198 | 2.5 |
| 2 | 2 | E(30):A(35):G(35) | NA | 53.8 | 2.03 | 95 | 0.44 | 186 | 2.0 |
| 10 | 3 | E(30):A(20):G(50) | 11 | 30.0 | 1.20 | 84 | 0.46 | 218 | 1.0 |
| 11 | 4 | E(30):A(35):G(35) | 11 | 30.5 | 1.27 | 128 | 0.50 | 157 | 2.2 |
| 12 | 5 | E(30):A(50):G(20) | 11 | 28.7 | 1.03 | 269 | 0.73 | 109 | 2.3 |
| 13 | 6 | E(30):A(35):G(35) | 11 | 23.8 | 1.05 | 230 | 0.65 | 110 | 2.3 |
| 14 | 7 | E(30):A(35):G(35) | 11 | 25.2 | 1.32 | 105 | 0.55 | 209 | 1.6 |
| 15 | 8 | E(30):A(65):G(5) | 11 | 31.4 | 1.28 | 165 | 0.50 | 122 | 2.8 |
| 16 | 9 | E(30):A(70) | 11 | 29.2 | 1.03 | 214 | 0.54 | 102 | 4.4 |
| 17 | 10 | E(40):A(60) | 11 | 31.4 | 1.24 | 178 | 0.81 | 183 | 4.5 |
| 18 | 11 | E(20):A(80) | 11 | 32.1 | 1.53 | 45 | 0.09 | 77 | 5.1 |
| 19 | 12 | E(30):A(50):G(20) | 5 | 61.5 | 1.48 | 157 | 0.56 | 144 | 2.2 |
| 20 | 13 | E(30):A(35):G(35) | 5 | 59.1 | 1.49 | 129 | 0.48 | 149 | 1.2 |
| 21 | 14 | E(30):A(35):G(35) | 5 | 56.6 | 1.51 | (156) | (1.01) | (661) | 1.6 |
| 22 | 15 | E(70):A(15):G(15) | 5 | 58.3 | 1.34 | 416 | 1.17 | 113 | 1.2 |
| 23 | 16 | D(80):A(20) | 8 | 13.5 | 1.08 | 613 | 1.32 | 86 | 2.8 |
| 24 | 17 | D(65):A(35) | 8 | 13.1 | 1.19 | 578 | 1.31 | 91 | 2.1 |
| 25 | 18 | D(50):A(50) | 8 | 13.0 | 1.08 | 524 | 1.43 | 109 | 2.7 |
| 26 | 19 | D(55):A(10):P(35) | 15 | 51.3 | 1.52 | 654 | 1.32 | 81 | 2.8 |
| 27 | 20 | D(35):A(30):P(35) | 15 | 48.8 | 1.54 | 572 | 1.29 | 90 | 2.9 |
| 78 | 60 | D(80):A(20) | 5 | 54.4 | 1.23 | 646 | 1.36 | 69 | 0.63 |
| 79 | 61 | D(65):A(35) | 5 | 53.8 | 1.15 | 601 | 1.48 | 81 | 0.86 |
| 80 | 62 | D(50):A(50) | 5 | 54.2 | 1.25 | 176 | 1.10 | 175 | 1.3 |
| 81 | 63 | E(30):A(30):G(30):D(10) | 5 | 58.4 | 1.10 | 255 | 0.72 | 90 | 0.78 |
| 82 | 64 | E(40):A(60) | 5 | 62.8 | 1.12 | 528 | 1.25 | 80 | 2.5 |
| 83 | 65 | D(5):A(5):G(90) | 5 | 54.1 | 1.45 | 7.7 | 0.04 | 228 | 2.7 |
| 84 | 66 | D(5):A(90):G(5) | 5 | 60.4 | 1.23 | 827 | 1.79 | 70 | 3.7 |
| 85 | 67 | D(90):A(5):G(5) | 5 | 60.6 | 1.31 | 266 | 0.27 | 32 | 3.0 |
| 86 | 68 | E(30):DAM(35):G(35) | 5 | 55.3 | 1.44 | 99 | 0.38 | 122 | 0.85 |
| 87 | 69 | E(10):A(60):D(30) | 5 | 61.4 | 1.38 | 545 | 1.72 | 126 | 0.13 |
| 88 | 70 | E(30):A(60):D(10) | 5 | 60.9 | 2.60 | 685 | 1.59 | 80 | 1.0 |
| 89 | 71 | E(20):A(60):D(20) | 5 | 58.1 | 2.54 | 673 | 1.59 | 81 | 3.0 |
| 90 | 72 | E(50):A(25):G(25) | 16 | 83.6 | 1.32 | 241 | 0.73 | 95 | 1.1 |
| Capto | Comp. | NA. Agarose based | NA | 88.3 | 2.22 | (143) | (3.75) | (2042) | None |
| Core | resin 1 | finished bead | | (d) | (d) | | | | |
| 700 (c) | | | | | | | | | |
| Oasis | Comp. | NA. D/P copolymer | NA | 26.8 | 1.86 | 806 (e) | 1.32 (e) | 83 (e) | None |
| HLB | resin 2 | | | | | | | | |
| Generik | Comp. | E/G copolymer | NA | 59.4 | 1.99 | 280 | 0.55 | 273 | None |
| MC | resin 3 | | | | | | | | |

E refers to EGDMA, A refers to AMA, G refers to GMA, D refers to DVB, and P refers to PVP, DAM refers to diallyl maleate in this table.
(a) Porous structure properties were measured using liquid N₂ adsorption and desorption method (BET and BJH). Values reported in parentheses were measured by mercury intrusion.
(b) Alkene content here is reported based on dry resin weight.
(c) Control sample, Capto Core 700 is a finished bead
(d) Measured by a light scattering method. All others were measured by a Beckman Coulter method.
(e) From resin COA data.

TABLE 3
Summary of properties of resins in Example 28-61 (Kinetic control method)

| | | | | Shell | | Chemistry |
|---|---|---|---|---|---|---|
| | | Mother | Shell | thickness | | procedure |
| Example | Resin | resin | chemistry | (μm) | Core chemistry | ref. |
| 28 | 20 | 4 | hydroxy | 1.7 | octylamine | GM1e |
| 29 | 21 | 4 | hydroxy | 1.7 | butylamine | GM1b |
| 30 | 22 | 5 | hydroxy | 1.7 | ethylamine | GM1a |
| 31 | 23 | 6 | hydroxy | 1.7 | MBA | GM1f |
| 32 | 24 | 12 | hydroxy | 3.4 | octylamine | GM1e |
| 33 | 25 | 12 | hydroxy | 3.4 | butylamine | GM1b |
| 34 | 26 | 13 | hydroxy | 3.4 | octylamine | GM1e |
| 35 | 27 | 13 | hydroxy | 3.4 | butylamine | GM1b |
| 36 | 28 | 14 | hydroxy | 3.4 | butylamine | GM1b |
| 37 | 29 | 14 | hydroxy | 3.4 | butylamine | GM1d |
| 38 | 30 | 14 | hydroxy | 3.4 | ethylamine | GM1a |
| 39 | 31 | 14 | hydroxy | 3.4 | butylamine | GM1c |
| 40 | 32 | 14 | hydroxy | 3.4 | TMA | GM1g |
| 41 | 33 | 15 | hydroxy | 3.4 | butylamine | GM1b |
| 42 | 34 | 1 | hydroxy | 1.7 | butylamine | GM1b |
| 43 | 35 | 18 | hydroxy | 1.7 | butylamine | GM1b |
| 44 | 36 | 20 | hydroxy | 2.2 | butylamine | GM1b |
| 45 | 37 | 13 | hydroxy | 3.4 | TMA | GM1g |
| 46 | 38 | 13 | hydroxy | 3.4 | sulfonate | GM1l |
| 47 | 39 | 13 | hydroxy | 3.4 | 1-hexanethiol | GM1m |
| 48 | 40 | 13 | hydroxy | 3.4 | 2-phenylethanethiol | GM1n |
| 49 | 41 | 13 | hydroxy | 3.4 | BMEA | GM1j |
| 50 | 42 | 13 | hydroxy | 3.4 | DMBA | GM1i |
| 51 | 43 | 13 | hydroxy | 3.4 | BMBA | GM1k |
| 52 | 44 | 13 | hydroxy | 3.4 | iminodiacetic acid | GM1h |
| 53 | 45 | 13 | hydroxy | 3.4 | HS-C8-25dT | GM1o |
| 54 | 46 | 13 | hydroxy | 3.4 | EDANS | GM1p |
| 55 | 47 | 13 | hydroxy | 3.4 | Congo Red dye | GM1q |
| 56 | 48 | 13 | hydroxy | 3.4 | 3-aminophenylboronic acid | GM1r |
| 57 | 49 | 13 | hydroxy | 3.4 | rSPA | GM1s |
| 58 | 50 | 12 | TMA | 3.4 | hydroxy | GM2a |
| 59 | 51 | 12 | TMA | 3.4 | 1-hexanethiol | GM2b |
| 60 | 52 | 12 | sulfonate | 3.4 | hydroxy | GM2c |
| 61 | 53 | 12 | sulfonate | 3.4 | 1-hexanethiol | GM2d |
| 91 | 73 | 14 | EDANS | | hydroxy (core), Congo red | GM1t |
| | | | (outer layer) | | (intermediate layer) | |
| 92 | 74 | 63 | hydroxy | 3.3 | butylamine | GM1b |
| 93 | 75 | 64 | hydroxy | 1.7 | Butylamine | GM1b |
| 94 | 76 | 65 | hydroxy | 3.0 | Butylamine | GM1b |
| 95 | 77 | 66 | hydroxy | 3.4 | Butylamine | GM1b |
| 96 | 78 | 67 | hydroxy | 3.4 | Butylamine | GM1b |
| 97 | 79 | 68 | hydroxy | 3.1 | Butylamine | GM1b |
| 98 | 80 | 69 | hydroxy | 3.4 | Butylamine | GM1b |
| 99 | 81 | 70 | hydroxy | 3.4 | Butylamine | GM1b |
| 100 | 82 | 71 | hydroxy | 3.3 | Butylamine | GM1b |
| 101 | 83 | 72 | hydroxy | 8.6 | Butylamine | GM1b |
| Capto | Comp. | | Dextran | 5.0 | Octylamine | |
| Core 700 | resin 1 | | (MW | | | |
| | | | 70,000 or | | | |
| | | | 500,000) | | | |
| Oasis | Comp. | | MBA | None | MBA | |
| MAX | resin 4 | | | | | |

TABLE 4
Summary of properties of resins in Example
62-67 (masking-unmasking method)

| Example | Resin | Mother resin | Shell chemistry | Core chemistry |
|---|---|---|---|---|
| 62 | 54 | 9 | hydroxy | diethylamine |
| 63 | 55 | 9 | sulfonate | TMA |
| 64 | 56 | 8 | hydroxy | TMA |
| 65 | 57 | 2 | hydroxy | sulfonate |
| 66 | 58 | 16 | hydroxy | TMA |
| 67 | 59 | 19 | hydroxy | sulfonate |
| 102 | 84 | 61 | hydroxy | EDANS |

TABLE 5
A summary of acronyms appeared in the present invention

| | |
|---|---|
| AIBN | azobisisobutyronitrile |
| AMA | allyl methacrylate |
| BA | butyl acrylate |
| BMA | benzyl methacrylate |
| CIP | clean in place |
| CLSM | confocal laser scanning microscopy |
| DAM | diallyl maleate |
| DDM | n-dodecyl-β-D-maltoside |
| DSP | downstream processing |
| DVB | divinylbenzene |
| EGDMA | ethylene glycol dimethacrylate |
| ELSD | evaporative light scattering detector |
| FPLC | fast protein liquid chromatography |
| GMA | glycidyl methacrylate |
| HCP | host cell proteins |
| HIC | hydrophobic Interaction chromatography |
| HILIC | hydrophilic interaction chromatography |
| HPLC | high-performance liquid chromatography |
| IDA | iminodiacetic acid |
| IEC | ion exchange capacity |
| IPA | isopropyl alcohol |
| LC | liquid chromatography |
| LNP | lipid nanoparticles |
| MW | molecular weight |
| NSB | nonspecific bonding |
| NTA | nitrilotriacetic acid |
| ODM | n-octyl-β-D-maltoside |
| PBA | phenyl boronic acid |
| PBMA | poly(benzyl methacrylate) |
| PEG | polyethylene glycol |
| PPG | polypropylene glycol |
| PS | polystyrene |
| PVP | polyvinylpyrrolidone |
| QC | quality control |
| RP | reverse phase |
| rt | room temperature |
| SAX | strong anion exchange |
| SCX | strong cation exchange |
| SDBS | sodium dodecylbenzene sulfonate |
| SDS | sodium dodecyl sulfate |
| SEC | size exclusion chromatography |
| SEM | scanning electron microscope |
| SPDS | solid phase DNA synthesis |
| SPOS | solid phase organic synthesis |
| SPPS | solid phase peptide synthesis |
| SSC | solid supported catalysts |
| TED | tri(carboxymethyl)ethylene diamine |
| Tg | glass transition temperature |
| VLP | virus-like particles |
| WAX | weak anion exchange |
| WCX | weak cation exchange |
| MBA | N, N-dimethylbutylamine |
| TMA | N, N, N-trimethylamine |
| DMBA | N, N-dimethylbenzylamine |
| BMEA | N-benzyl-N-methylethanolamine |
| BMBA | 2-benzamido-4-mercaptobutanoic acid |
| HS-C8-25dT | 8-thio-octyl-oligo(dihydrothymine) with 25 nt |

### Discussion

As known for preparative purification, conventional resins with broad size distribution have the following disadvantages: 1) Common request for removing small beads (fines), which results in inefficient manufacturing, waste generation and its high disposition cost. 2) High column back pressure, which results in limitations of flow rate, long purification process time, low purification throughput, and high manufacturing cost. 3) Difficulties in packing or repacking columns with high performance or great consistence. 4) Unstable column bed, which is subject to change under high flow or highly pressured operation conditions, so constant column maintenance or repacking may be required. 5) Large elution volume, which produces diluted purification fractions. Thus, there is a demand in making chromatography media with narrow size distribution consistently lot-to-lot.

However, synthesis of polymeric porous resins with narrow size distribution is hard to achieve and requires high synthetic skills. Seed polymerization process provides a solution to narrow-dispersed resin synthesis. It was first demonstrated by Ugelstad in 1970's, and was further developed (and redeveloped), and commercialized by many companies (Dynal, Polymer Laboratories Ltd, Tosoh) and academic groups (Mohamed El-Aasser, Jean Frechet) in 1980's and 1990's. However, said seed polymerization process can only provide resins with conventional porous structure, which are lack of functional groups on pore wall. Said drawbacks limit their broad applications as LC media.

In the present invention, synthesis of mother resin with narrow size distribution was realized through sequential seed polymerization processes via a shape template, using either low MW polymeric (or oligomeric) seeds or oil droplets, which are water insoluble, and swellable with monomer(s) used in later seed polymerization process. Said low MW polymeric (oligomeric) seeds made in a suspended solution can be used in-situ for the next stage seeding process. Alternatively, a primary seed if made into a high Tg (glass transition temperature) polymer (well above rt, like ≥40° C.) can be separated and collected, stored, and redispersed to make a seed solution and used thereafter.

In the present invention, the size of said seeds can be controlled through the following parameters, such as type and concentration of initiator, choice of water insoluble monomer(s), type and concentration of surfactant, stirring speed, size/shape/number/location of impeller, reactor geometry, seeds used in the immediate prior stage, as well as a swelling ratio (weight of monomers in the later seed polymerization vs. weight of said polymeric (or oligomeric) seeds).

In the present invention, through unremitting optimizations of said conditions, average particle diameter of said porous resins can be made with a desired size selected in a range of 1 μm to 1000 μm, it is more preferred from 1 μm to 500 μm, most preferred from 2 μm to 200 μm.

Meanwhile, particle size distribution (D₉₀/D₁₀), which was used to assess resin quality in terms of particle uniformity, for a medium made from conventional emulsion polymerization can be controlled to be ≤2.2, preferred ≤2.0, while for a medium made from seeding process can be controlled to be ≤1.6, preferred ≤1.5, more preferred ≤1.2.

Capto® Core multimodal chromatography resins with core-shell hierarchical structure were developed and commercialized by GE Healthcare (as shown in Table 2), which is designed for purifying intermediate bio-products and polishing of viruses and other large biomolecules. These chromatographic adsorbent particles have an inert size-selective shell and an adsorptive core, which can simplify the purification processes through combining adsorptive and size-exclusion mechanisms. The porous structure is distinctly different between exterior surface and shell layer (tight pores) and core of the medium (open/large pores). Many successful purification processes of biomolecules, such as proteins, viruses, etc., were achieved by using these resins.

However, agarose-based beads are generally soft and easily crushed, so they are limited to be used under gravity-flow, or low-pressure conditions. Column bed is not stable and is subject to change under high flow or highly pressured operation conditions, so constant column maintenance or repacking may be required. Even through the strength of the resins can be improved by increased cross-linking, such change may also result in a lower binding capacity in some separation processes.

Capto™ Core resins, agarose-based beads with ˜85 μm averaged diameter, could be deformed under pressured conditions. Said resins are polydispersed in a range of 50-130 μm, and have a broad pore size distribution according to the SEM studies, even though their shell can be further modified with Dextran, the uniformly chemical modification is hardly achieved. Furthermore, harsh CIP (clean in space) conditions, such as 1 M NaOH in 30% IPA (isopropyl alcohol) aqueous solution, are required to remove some substances with similar size stuck in the pores, this limitation results in high economy and time cost, as well as short lifetime of said resins.

Said Capto™ core resins also have some other limitations. For example, 1) octylamine is the only ligand used in commercialized products currently, which may not meet all the biomolecule separation needs; 2) the shell chemistry is also only limited to hydroxyl groups (—OH) and Dextran; 3) the pore size is only limited to two choices, wherein Core 700 and Core 400 have MW exclusion limit up to 700 KDa, and 400 KDa, respectively, and is still too large to some molecules. This limits its applications in separating molecules under 400 KDa; 4) the control of functional group density on both shell and core has not yet been demonstrated. Said drawbacks greatly limit the applications of said Capto™ core resins.

In the present invention, it provides a novel hierarchical structural LC medium (or chromatography medium) made from a mother medium, which is formed through copolymerization of multiple monomers, wherein the chemical or physical property of said mother resin is tunable through choosing variety of monomers carrying desired functional group(s), and adjusting the ratio of monomers, such as mechanical strength, hydrophilicity, hydrophobicity, hydrogen bonding, affinity, π-π interaction, electrostatic force, and Van der Waals force, etc.

In particular, the present invention provides the following technical solutions (TS):
TS1. a synthetic polymeric porous medium (1) with hierarchical multiple layer structure and essentially homogeneous porous structure from inside to outside of the medium for LC applications, which is made from a mother medium (2), with robust chemical and physical stability, and promising physicochemical properties, copolymerized from the following monomers: at least one crosslinking monomer (3), at least one monomer (4) with designed functional group further used for hierarchical structure construction, and optional monomer(s) (5) carrying special functional group(s) for tuning its properties, said mother medium is further chemically modified (6) to a finished medium with a distinct core-shell(s) structure (7).
wherein the synthetic polymeric porous mother medium has
(a) specific pore volume in a range of 0.05 to 3.0 mL/g,
(b) specific surface area in a range of 40-1200 m²/g,
(c) average pore size in a range of 70-5000 Å, and preferably, the average pore size is essentially homogeneous from inside to outside of the porous mother medium,
(d) volume average particle diameter (D₅₀) in a range of 1-1000 μm,
(e) particle size distribution (D₉₀/D₁₀) in a range of 1.0-2.2,
(f) alkene content of the mother medium in a range of 0.5-6.0 mmol/g.
TS2. The chromatography medium of TS 1, wherein the preferred shape and form of said LC medium is a substantially flat particulate or monolithic rod or disk, the most preferred shape of a particulate is spherical or pseudo-spherical and wherein the porous structure from inside to outside of the porous medium is essentially homogeneous and.
TS3. The chromatography medium of TS 1 (2), wherein the physicochemical properties of said mother medium are tunable through choosing different types of monomers carrying desired functional group(s), and adjusting the ratio of monomers, such as hydrophilicity, hydrophobicity, hydrogen bonding, affinity, π-π interaction, electrostatic force, and Van der Waals force, etc.,
TS4. Said crosslinking monomer of TS 1(3) accounts for 1-99% wt of total monomers used in copolymerization process. In one preferred embodiment, said crosslinking monomers are (meth)acrylic, styrenic, and other vinylic monomers, such as divinylbenzene (DVB), ethylene glycol dimethacrylate, pentaerythritol dimethacrylate, pentaerythritol triacrylate, pentaerythritol tetraacrylate, sorbitol dimethacrylate, poly(ethylene glycol) diacrylate, poly(propylene glycol) diacrylate, trimethylolpropane triacrylate, bis2-(methacryloyloxy)ethyl phosphate, N, N′-methylenebisacrylamide, 3-(acryloyloxy)-2-hydroxypropyl methacrylate, glycerol 1,3-diglycerolate diacrylate, 1,5-hexadiene, allyl ether, diallyl diglycol carbonate, di(ethylene glycol) bis(allyl carbonate), ethylene glycol bis(allyl carbonate), triethylene glycol bis(allyl carbonate), tetraethylene glycol bis(allyl carbonate), glycerol tris (allyl carbonate), ethylene glycol bis(methallyl carbonate), diallyl phthalate, triallyl isocyanurate, diallyl isophthalate, diallyl terephthalate, diallyl itaconate, diallyl 2,6-naphthalene dicarboxylate, diallyl chlorendate, triallyl trimellitate, triallyl citrate, 2,4,6-triallyloxy-1,3,5-triazine, 1,3,5-triacryloylhexahydro-1,3,5-triazine, glyoxal bis(diallyl acetal), N,N-diallyldimethyl ammonium salts, ethylene glycol diallyl ether, etc.
TS5. Said monomer of TS 1(4) accounts for 1-99% wt of total monomers used in copolymerization process, and can be any one or more of the following, including (meth)acrylate, acrylamide, ethylene terephthalate, ethylene, propylene, styrene, vinyl acetate, vinyl acrylate, vinyl chloride, vinyl pyrrolidone, DVB, 1,3,5-trivinylbenzene, and their derivatives, etc.
TS6. Said monomer of TS 5 should contain at least one inactive, less active, or protected functional group(s) which can survive during polymerization process, and then further be used in hierarchical modification directly or indirectly. Said functional group(s) can be amino, sulfenyl, benzyl, phenyl, alkyl, alkynyl, hydroxyl, carboxyl, aldehyde, halo, thiol groups, etc. In the preferred embodiment, the reactive group is alkene like allyl, vinyl, and other groups with carbon-carbon double bonds, said alkenyl group can be from (meth)acrylic, styrenic, and other vinylic monomers agents, such as allyl acrylate, vinyl acrylate, diallyl maleate, DVB, 1,3,5-trivinylbenzene, etc. Said alkenyl group can also be selected from monomers in TS 4. The most preferred monomers are allyl methacrylate and diallyl maleate.
TS7. Said monomer of TS 1(5) accounts for 1-99% wt of total monomers used in copolymerization process, which can render desired property to the mother medium, can be any one or more of the following, such as (meth)acrylic, styrenic, and other vinylic monomers compounds, glycidyl methacrylate, 2-hydroxyethyl methacrylate, 2-carboxyethyl acrylate, 2-hydroxyethyl acrylate, 2-hydroxyethyl methacrylate, methyl methacrylate, methacrylic acid, hydroxypropyl methacrylate, 2-(methacryloyloxy)ethyl acetoacetate, mono-2-(methacryloyloxy)ethyl maleate, benzyl acrylate, butyl acrylate, styrene, DVB, N-vinylpyrrolidone, etc.
TS8. Distinct core-shell(s) structure of TS 1(7) has at least two layers, where the core refers to the most inner layer, while the shell(s) refer(s) to the outer layer(s) from the core. Core and each shell layer are spatially defined with distinct functional groups and relative spatial layout between each layer. One of the preferred said hierarchical structures is core-shell (two-layer) structure with chemically distinct functional groups or same functional group with different density in each layer.
TS9. Said each distinct layer of TS 8 can have any suitable functional group(s), which could render such layer LC separation mechanism chosen from SEC, SAX, WAX, SCX, WCX, HIC, affinity, or mixed-mode.
TS10. Distinct core-shell(s) structure of TS 8 can be experimentally visualized by using a tag such as a fluorescent dye, or a tracer such as a fluorescent tagged globular protein through covalently binding or physicochemical interaction, such as hydrophobic, IEX, affinity and others.
TS11. The core-shell structure of TS 8, wherein said LC medium can be modified to a hydrophilic shell and cationic ligand(s) activated core with or without a linker. Said cationic ligand(s) is preferred to be ammonium, sulfonium, phosphonium, or other groups, such as primary amines, preferred ethylamine, butylamine, hexylamine, octylamine, or secondary amines, preferred dimethylamine, diethylamine, or tertiary amines, preferred trimethylamine, N,N-dimethylbutylamine, etc.
TS12. The core-shell structure of TS 8, wherein said LC medium can be modified to a hydrophilic shell and anionic ligand(s) activated core with or without a linker. Said anionic ligand(s) can be any suitable sulfonate, phosphate, carboxylate, and their derivatives.
TS13. The core-shell structure of TS 8, wherein said LC medium can be modified with a hydrophilic shell and hydrophobic ligand(s) activated core with or without a linker. Said hydrophobic ligand(s) can be any suitable hydrophobic group(s) linked to the backbone through oxygen atom (O), nitrogen atom (N), sulfur atom (S), ether, ester, or amide groups, such as linear or branched alkyl chain (C1-C18), oligo(ethylene oxide), phenyl, benzyl groups, and their derivatives.
TS14. The core-shell structure of TS 8, wherein said LC medium can be modified to a hydrophilic shell and affinity ligand(s) activated core with or without a linker. Said affinity ligand(s) can be any ligand, or any suitable one with a strength of binding interaction to its binding partner, such as Protein A, 3-aminophenylboronic acid, and sense/antisense oligonucleotide, etc. Said ligand can also be iminodiacetic acid (IDA), tri(carboxymethyl)ethylene diamine (TED), nitrilotriacetic acid (NTA), and other metal chelating ligands.
TS15. The core-shell structure of TS 8, wherein said LC medium can be modified with a hydrophilic shell and mixed-mode ligand(s) activated core with or without a linker. Said mixed-mode ligand, a stationary phase ligand composed of at least one hydrophobic moiety at peripheral position or branch position, and at least one ionic or ionizable groups at peripheral position or branch position, or embedded in the hydrophobic moiety, can be any suitable mixed mode ligand, such as alkylamine (like hexylamine, octylamine, etc.), N, N-Dimethylbutylamine, N-Benzyl-N-methylethanolamine, N, N-Dimethylbenzylamine, and 2-benzamido-4-mercaptobutanoic acid, etc.
TS16. The medium with core-shell structure of TS 8 and its corresponding LC applications, wherein said medium has a cationic shell, which can be modified with any suitable reagent leading to positively charged ligands, and a hydrophobic ligand(s) activated core, which can carry any hydrophobic ligands.
TS17. The medium with core-shell structure of TS 8 and its corresponding LC applications, wherein said medium has an anionic shell, which can be modified with any suitable reagent leading to negatively charged ligands, and a hydrophobic ligand(s) activated core, which can carry any hydrophobic ligands.
TS18. The LC medium with core-shell structure of TS 8 and its corresponding LC applications, wherein said medium has an ionic or ionizable shell, which can be modified with any suitable reagent leading to said ionic or ionizable ligands, and a hydrophilic core.
TS19. The LC medium of TS 8, wherein said medium can be modified with the same ligand(s) in both the core layer and the shell layer, but with a different functional group(s) density. Wherein said ligand(s) can be any mentioned in TS 11-15.
TS20. The LC medium of TS 11-15 and 18, wherein the hydrophilicity in each shell of said medium can be tuned and enhanced through chemical modification with 2-hydroxyethanethiol, 3-sulfanylpropane-1,2-diol, Dextran, any linear or branched multifunctional epoxide, or any other agents with hydrophilic functional groups.
TS21. Specific pore volume of TS 1 (a) ranges from 0.05 to 3.0 mL/g, preferably from 0.2 to 2.5 mL/g, most preferably from 0.4 to 2.0 mL/g.
TS22. Specific surface area of TS 1 (b) ranges from 40 to 1200 m²/g, preferably from 60 to 1000 m²/g, most preferably from 80 to 800 m²/g.
TS23. Average pore size of TS 1(c) ranges from 30 Å to 5000 Å, more preferably from 50 Å to 3000 Å, most preferably from 100 Å to 2000 Å; and preferably, the average pore size is essentially homogeneous from inside to outside of the porous mother medium.
TS24. Volume average particle diameter (D₅₀) of TS 1(d) ranges 1 μm to 1000 μm, more preferably from 1 μm to 500 μm, most preferably from 2 μm to 200 μm.
TS25. Particle size distribution (D₉₀/D₁₀) of TS 1(e) for a medium made from conventional emulsion polymerization is ≤2.2, prefer ≤2.0, for a medium made from seed polymerization process is ≤1.6, prefer ≤1.5.
TS26. Alkene content from the mother medium of TS 1(f) ranges from 0.5 to 6.0 mmol/g, preferably from 0.7 to 5.5 mmol/g, most preferably from 0.9 to 5.2 mmol/g.
TS27. The porous structure of TS 1 such as pore size, surface area, pore volume, pore connectivity, surface pore morphology (smooth vs. rough) is controlled by the choice(s) of porogens, and weight ratio of total amount of porogens to total amount of monomers, and weight ratio of a specific porogen.
TS28. Suitable porogens of TS 27 can be chosen from: 1) a conventional solvent such as hexanes, pentanes, octanes, pentanols, hexanols, heptanols, octanols, methyl isobutyl carbinol, cyclohexanol, toluene and xylenes, ethyl acetate, diethyl phthalate, and dibutyl phthalate; 2) oligomer such as polypropylene glycol (PPG), and polyethylene glycol (PEG); 3) a swellable polymer/oligomer seeds, a oligomer made from (meth)acrylic, styrenic, and other vinylic monomers such as oligostyrene, oligoacrylates, oligo-BMA, oligo-BA, vinyl acetate, or any of their mixtures.
TS29. The weight ratio of total amount of porogens chosen from TS 27 to total amount of monomers is 10%-400%, preferred 20-350%, more preferred 30-300%, most preferred 50-250%. If a mixture of porogens is chosen, the weight ratio of one single porogen to total weight of porogen is 0.1%-99.9%, preferred 1%-99%, more preferred 3%-97%, most preferred 5%-95%.
TS30. The chromatography medium of TS 1, wherein monosized bead size distribution is controlled and achieved through sequential seed polymerization process using a swellable polymeric (oligomeric) seeds which are essentially water insoluble and swellable with monomer(s), porogen(s) and solvent used in later seeding process. (a) Said low MW polymer (oligomer) made in a suspended solution can be used in-situ for the next stage seeding process; (b) alternatively, a primary seed if made into a high Tg polymer (well above rt, like 40° C.) can be separated and collected, stored, and redispersed to make a seed solution and used thereafter; (c) the size of seeds is controlled by the polymerization parameters (initiator type and concentration, choice of water insoluble monomer(s), surfactant type and concentration, stirring speed, size/shape/number/location of impeller, and reactor geometry) for primary seeds in (a-b) and weight ratio of the monomers in the later seed polymerization to early polymeric (oligomeric) seeds.
TS31. In TS 30, water insoluble monomer(s) can be one or more monomers that are miscible and can form a later stage seed without inducing macrophase separation. Choice of monomers can be water insoluble acrylate or vinyl monomer with up to 2 wt % crosslinker like diacrylate or divinyl monomer. It is preferred that no crosslinker is used in every stage of seeds preparation. The preferred monomers are (meth)acrylic, styrenic, and other vinylic monomers compounds, such as benzyl methacrylate, butyl acrylate, styrene and their binary and tertiary mixture.
TS32. In TS 30, the seeds have a low MW and are swellable by monomers and porogens used in later stage seeding process. MW is less than 70,000 g/mol for primary seeds and 10,000 g/mol for later stage seeds. More preferred MW is less than 30,000 g/mol for primary seeds and 5,000 g/mol for later stage seeds. Seeds can be pre-swelled with treating a desired solvent, which is soluble with seeds but less soluble or insoluble with water, to enhance swellability.
TS33. In TS 30, excessive initiator (inorganic peroxide, organic peroxide, azo type initiators), excessive chain transfer reagent (thiol containing molecules) or their combination is used to keep seeds MW low and enable seeds to acquire high swellability. Preferred to have >0.5 wt % initiator and/or >1 wt % chain transfer reagent with respect to weight of polymerizable monomer(s) is used. More preferred to have >1.0 wt % initiator and/or >3 wt % chain transfer reagent.
TS34. Seeding TS 30, the swelling ratio in each seeding process is preferred to be 2-300, more preferred 5-200, even more preferred 10-100, most preferred 20-80.
TS35. The hierarchical structured medium of TS 1 (6) can be constructed through chemical kinetic/diffusion control of alkene bromination.
TS36. The shell thickness of the LC medium of TS 35 is tunable with the amount of bromine used in partial bromination step.
TS37. The functional group density on both the shell layer and the core layer of said medium of TS 35 is tunable based upon the needs of a specific LC separation.
TS38. The LC medium of TS 1(6), wherein said hierarchical structured medium can be made through a masking-unmasking (protection-deprotection) process with inert fillings.
TS39. The inert fillings of TS 38, wherein said inert fillings can be in liquid, gel/semi-solid or solid, regardless of their molecular weight and sizes. More preferably, the inert filling in gel/semi-solid or solid form. Most preferably, the inert filling is in solid form which will remain inside the pores throughout chemical transformations at the selective layers.
TS40. The inert fillings of TS 38, wherein said inert fillings used will be 1-300% weight of porous resin, preferably 3-200%, most preferably 5-150%, depending on the pore wall surface area needs to be masked.
TS41. The inert fillings of TS 38, wherein solid inert filling will not melt up to 200° C., preferably, the inert filling will remain solid at 20° C.-150° C.
TS42. The LC medium of TS 1 can be physically converted/transformed into LC columns or other confined devices for molecular separations and purifications. Specifically, LC columns or devices can be analytical columns, guard columns, preparative columns, semi-prep columns, HPLC columns, UPLC columns, UHPLC columns, FPLC columns, flash columns, gravity columns, capillary columns, spin columns, disposable columns, monolithic columns, extraction cartridges and plates, etc.
TS43. The LC columns or devices of TS 42: a) can be applied in batch mode or continuous mode such as counter current chromatography; b) has ID from 0.1 millimeter to 2 meters and any length from 1 millimeter to 2 meters. Said column or disc housing material can be stainless, PEEK, glass or borosilicate glass, or other synthetic polymeric materials such HDPE (high density polyethylene); 3) can be used as single column or multi-column format in continuous or non-continuous (conventional) chromatography.
TS44. A LC medium of TS 1 with core-shell two-layer structure and designed pore size is used for analytical and preparative separations. Said medium combines size exclusion separation and various binding chemistry, wherein larger molecules are excluded from the no-binding shell and analyzed or collected as flow-through, while smaller molecules penetrate through the pore, and are temporarily trapped/bound into the functionalized core of the separation medium, which can be analyzed or collected in a bind-elute mode later. Here the separation sample comprising at least two substances with distinct molecular weight. Molecular weight ratio M1/M2 ≥2; preferably M1/M2≥5, most preferably M1/M2≥10, where M1 refers to the largest substance, and M2 refers to the smallest substance in the separation mixture. Such a LC medium can be packed into a LC column for LC applications.
TS45. The LC medium and column of TS 11 and 44, wherein said medium combines anionic exchange adsorptive and size exclusion mechanisms, where the large substance, such as large protein, virus, large DNA, can be analyzed or collected in flow-through mode, while the small substances are temporarily trapped/bound in the core, and then eluted for analysis or collection.
TS46. The LC medium and column of TS 12 and 44, wherein said medium combines cationic exchange adsorptive and size exclusion mechanisms, where the large substance, such as large protein, virus, large DNA, can be analyzed or collected in flow-through mode, while the small substances are temporarily trapped/bound in the core, and then eluted for analysis or collection.
TS47. The LC medium and column of TS 13 and 44, wherein said medium combines hydrophobic adsorptive and size exclusion mechanisms, where the large substance, such as large protein, virus, large DNA, can be analyzed or collected in flow-through mode, while the small substances are temporarily trapped/bound in the core, and then eluted for analysis or collection.
TS48. The LC medium and column of TS 14 and 44, wherein said medium combines affinity adsorptive and size exclusion mechanisms, where the large substance, such as large protein, virus, large DNA, can be analyzed or collected in flow-through mode, while the small substances are temporarily trapped/bound in the core, and then eluted for analysis or collection.
TS49. The LC medium and column of TS 15 and 44, wherein said medium combines mixed-mode adsorptive and size exclusion mechanisms, where the large substance, such as large protein, virus, large DNA, can be analyzed or collected in flow-through mode, while the small substances are temporarily trapped/bound in the core, and then eluted for analysis or collection.
TS50. The LC medium and column of TS 44, wherein said medium can be applied to separate biomolecules from the surfactants used in stabilizing biotherapeutic formulation. Said preferred biomolecules can be therapeutic proteins with molecular weight range from 10 KD to 3 MD. Said surfactants can be polysorbates including Tween 20, 40, 60, and 80, polyethyleneoxide, poly(propylene oxide), sorbitan esters, ethoxylates, PEG, Poloxamer 188, Trion X-100, Miglyol, and maltosides including n-dodecyl-β-D-maltoside (DDM), n-octyl-β-D-maltoside (ODM).
TS51. The LC medium and column of TS 44, wherein said medium be applied to separate mixtures of large or super biomolecule assemblies natural or artificially-made such as eukaryotic and prokaryotic cells, VLPs, vaccines, viral vectors, viruses, or liposome or LNPs (lipid nano-particles) from small molecules or assemblies via the interactions at inner core different from that at outer layer.
TS52. In TS 51, said large or super biomolecule assemblies such as eukaryotic and prokaryotic cells, VLPs, vaccines, viruses, viral vectors, or liposomes are in the in size >10 nm. Said Virus can be active or inactivated, enveloped or nonenveloped. Such VLPs, vaccines, viruses, viral vectors, or liposome or LNPs (lipid nano-particles) can encapsulate genetic materials such as ssDNA, dsDNA, ssRNA, dsRNA. Such Liposome and lipid nanoparticle (LNP) can carry positive charge or negative charge or no charge, preferred entity carries positive charge.
TS53. Said small molecule or assemblies of TS 51 includes but not limited to DNA fragment, RNA, plasmids, HCP, protein fragments, capsid proteins, endotoxins, detergents, benzonase, excessive components (unencapsulated components), with a size <10 nm.
TS54. The LC medium and column of TS 44 bearing an affinity ligand Protein A attached an inner core, can be applied to separate a mixture of Fc containing proteins.
TS55. The LC medium and column of TS 44 bearing an affinity ligand Protein L attached an inner core, can be applied to separate a mixture of Fab or kappa light chain containing proteins.
TS56. The LC medium and column of TS 44 bearing an affinity ligand Protein G attached an inner core, can be applied to separate a mixture of Fc and Fab containing proteins.
TS57. The LC medium and LC column of TS 44 bearing an affinity ligand such as oligonucleotide dTs, with a length ranging from 5 to 50, more preferred 10-40, most preferred 20-30, can be applied to separate a mixture of oligonucleotide with polyA tag, such as vitro transcribed mRNA bearing polyA. The length of said mRNA is of 30-4000 nt, preferable, 100-2000 nt.
TS58. The LC medium and column of TS 44, enables a selective separation of lipid, proteins, and LNP encapsulated therapeutic biologic excluded from the shell, free therapeutic biologic is captured and released through its interaction with the core functional groups through an affinity, IEX or HIC mechanism. Biologic encapsulation yield, a ratio of encapsulated therapeutic biologic to the total of therapeutic biologic (encapsulated and free) in a mixture, and thus can be obtained.
TS59. In a special case of TS 58, mRNA encapsulation yield is calculated as a ratio of mRNA encapsulated in LNP to the total of mRNA encapsulated and free mRNA. LNP encapsulated mRNA is excluded from the medium shell (with hydroxy groups), and free mRNA is captured and then released by its affinity interaction with the dT functional groups in core.
TS60. The hierarchical structured resin of TS 10, said resin can be applied to make fluorescently labeled microparticles with hierarchical structure, wherein a fluorescent dye can label any selective layer of said microparticle.
TS61. The hierarchical structured resin of TS 10, wherein said resin can be applied to solid support with hierarchical structure used for solid phase synthesis, such as solid phase peptide synthesis (SPPS), solid phase DNA synthesis (SPDS), solid phase organic synthesis (SPOS).
TS62. The hierarchical structured resin of TS 10, wherein said resin can be applied to solid support with hierarchical structure used for development of solid supported catalysts (SSC), including organic SSC, inorganic SSC, and enzyme SSC.
TS63. In the copolymerization process, monosized bead size distribution is controlled and achieved through sequential seed polymerization process using a swellable polymeric (oligomeric) seeds which are essentially water insoluble and swellable with monomer(s), porogen(s) and solvent used in later seeding process. (a) Said low MW polymer (oligomer) made in a suspended solution can be used in-situ for the next stage seeding process; (b) alternatively, a primary seed if made into a high Tg polymer (well above rt, like ≥40° C.) can be separated and collected, stored, and redispersed to make a seed solution and used thereafter; (c) the size of seeds is controlled by the polymerization parameters (initiator type and concentration, choice of water insoluble monomer(s), surfactant type and concentration, stirring speed, size/shape/number/location of impeller, and reactor geometry) for primary seeds in (a-b) and weight ratio of the monomers in the later seed polymerization to early polymeric (oligomeric) seeds.
TS64. The method of TS63, wherein the water insoluble monomer is selected from the group consisting of one or more monomers that are miscible and can form a later stage seed without inducing macrophase separation. Choice of monomers can be water insoluble acrylate or vinyl monomer with up to 2 wt % crosslinker like diacrylate or divinyl monomer. It is preferred that no crosslinker is used in every stage of seeds preparation. The preferred monomers are such as (meth)acrylic, styrenic, and other vinylic monomers compounds, such as benzyl methacrylate, butyl acrylate, styrene and their binary and tertiary mixture.
TS65. The seeds of TS63 have a low MW and are swellable by monomers and porogens used in later stage seeding process. MW is less than 70,000 g/mol for primary seeds and 10,000 g/mol for later stage seeds. More preferred MW is less than 30,000 g/mol for primary seeds and 5,000 g/mol for later stage seeds. Seeds can be pre-swelled with treating a desired solvent, which is soluble with seeds but less soluble or insoluble with water, to enhance swellability.
TS66. Excessive initiator (inorganic peroxide, organic peroxide, azo type initiators), excessive chain transfer reagent (thiol containing molecules) or their combination is used to keep seeds of TS63 MW low and enable seeds to acquire high swellability. Preferred to have >0.5 wt % initiator and/or >1 wt % chain transfer reagent with respect to weight of polymerizable monomer(s) is used. More preferred to have >1.0 wt % initiator and/or >3 wt % chain transfer reagent.
TS67. the swelling ratio in each seeding process is 2-300, preferably 5-200, more preferably 10-100, most preferably 20-80.

Compared with the conventional LC medium, the synthetic polymeric porous chromatography medium of the present invention has achieved significantly increased separation properties at a relatively low cost which can efficiently promote the development of biological products.

The present invention also provides successful LC applications in biomolecule separation.

An object of the present invention is to provide an application of a synthetic medium with essentially homogeneous porous structure from inside to outside of the medium, which can be further modified to a core-shell structured medium for the purification and separation of viruses, virus vectors, and virus-like particles.

In the first aspect of the present invention, it provides a liquid chromatography method for purifying and separating viral antigens, and the method comprises the following steps:
1) providing a chromatography medium, viral antigen to be separated, first buffer, second buffer, and cleaning in place (CIP) solution;
wherein the chromatography medium is a synthetic hydrophilic polymer, has a porous structure, and has a 2-5 layered structures;
2) packing the liquid chromatography column with the chromatography medium, and the liquid chromatography column using the above method is obtained;
3) rinsing the liquid chromatography column with the first buffer;
4) loading the viral antigen to be separated into the liquid chromatography column obtained in step 3);
5) rinsing the liquid chromatography column obtained in step 4) with the second buffer, collecting the separated product to obtain the separated viral antigen;
6) rinsing the liquid chromatography column obtained in step 5) with the CIP solution, collecting the separated product, and removing the process-related impurities in the viral antigen.

In another preferred embodiment, the polymerizable monomer for the synthetic hydrophilic polymer is selected from the group consisting of (meth) acrylic monomers, styrene monomers, vinyl monomers, or combinations thereof.

In another preferred embodiment, the porous structure is used for size exclusion separation; and
at least one inner layer and at least one outer layer of the chromatography medium have different types of binding functional groups, or at least one inner layer and at least one outer layer of the chromatography medium have the same type of binding functional groups with different binding densities, such that at least one inner layer and at least one outer layer of the chromatography medium have different chromatographic properties.

In another preferred embodiment, the binding functional group is selected from the group consisting of hydrophilic group, hydrophobic group, ionic group, affinity group, mixed mode functional group.

In another preferred embodiment, the hydrophilic group is selected from the group consisting of hydroxyl groups, or groups converted by chemical modification of 2-hydroxyethanethiol, 3-sulfanylpropane-1-2-diol, dextran, any linear or branched polyfunctional epoxide.

In another preferred embodiment, the hydrophobic group is selected from the group consisting of linear or branched C1-C18 alkyl, oligo (ethylene oxide), phenyl, benzyl and derivatives thereof; preferably, the hydrophobic group is connected to the layer structure by an oxygen atom (O), a nitrogen atom (N), a sulfur atom (S), an ether, an ester or an amide group.

In another preferred embodiment, the ionic group is selected from the cationic group consisting of primary aminos, secondary aminos, tertiary aminos, or combinations thereof.

In another preferred embodiment, the primary amino is a linear or branched C1-C18 alkylamino; more preferably, the primary amino is selected from the group consisting of ethylamino, butylamino, hexylamino, octylamino, or a combination thereof.

In another preferred embodiment, the secondary amino is selected from the group consisting of dimethylamino, diethylamino, or a combination thereof.

In another preferred embodiment, the tertiary amino is selected from the group consisting of trimethylamino, N,N-dimethylbutylamino, or a combination thereof.

In another preferred embodiment, the ionic group is selected from the anionic group consisting of sulfonic groups, phosphate groups, carboxylic groups and derivatives containing related groups.

In another preferred embodiment, the affinity group is selected from the group consisting of protein A, protein L, protein G, 3-aminophenylboronic acid, positive-sense/anti-sense oligonucleotides, iminodiacetic acid (IDA), tris (carboxymethyl) ethylenediamino (TED), nitrilotriacetic acid (NTA) and other metal chelating ligands.

In another preferred embodiment, the mixed mode functional group is a secondary and tertiary amino containing at least one linear C2-C10 alkyl, N,N-dimethylbutylamino, N-benzyl-N-methylethanolamino, N,N-dimethylbenzylamino and 2-benzoylamino-4-mercaptobutyric acid.

In another preferred embodiment, the chromatography medium has a core-shell structure.

In another preferred embodiment, the chromatography medium has one or more features selected from the following group:
1) the specific pore volume of the chromatography medium is 0.05 mL/g-3.0 mL/g;
2) the specific surface area of the chromatography medium is 40 m²/g-1200 m²/g;
3) the pore size of the chromatography medium is 30 Å-5000 Å; and preferably, the average pore size is essentially homogeneous from inside to outside of the porous mother medium;
4) the volume average particle size of the chromatography medium is 1 μm-1000 μm;
5) the particle size distribution (D₉₀/D₁₀) of the chromatography medium is 1.0-2.2.

In another preferred embodiment, the shell thickness of the chromatography medium is between 0.5%-30% of the equivalent radius of the chromatography medium.

In another preferred embodiment, the shell thickness of the chromatography medium is 0.5 μm-10 μm;

In another preferred embodiment, when the functional group of the core layer is the same as the functional group of the shell layer, the functional group density of the core layer is D1, the functional group density of the shell layer is D2, and the chromatography medium has one of the following characteristics:
1) D1/D2 is greater than 1.05, preferably 1.1, more preferably 1.5, most preferably 2.0;
2) D2/D1 is greater than 1.05, preferably 1.1, more preferably 1.5, most preferably 2.0.

In another preferred embodiment, the chromatography medium is spherical or pseudo-spherical.

In another preferred embodiment, the liquid chromatography column has one or more features selected from the following group:
1) the ion exchange equivalent of the liquid chromatography column chromatography medium in the core layer is 100-500 μmol/mL;
2) the linear flow rate of the liquid chromatography column is 10-1000 cm/h;
3) the operating pressure of the liquid chromatography column is ≤100 bar.

In another preferred embodiment, the liquid chromatography column has an ion exchange equivalent in the core layer of 100-300 μmol/mL chromatography medium.

In another preferred embodiment, the linear flow rate of the liquid chromatography column is in a range of 20-900 cm/h; preferably 50-800 cm/h; more preferably 100-700 cm/h; most preferably 150-500 cm/h.

In another preferred embodiment, the operating pressure of the liquid chromatography column is ≤50 bar, preferably ≤10; preferably ≤5 bar; most preferably ≤3 bar.

In another preferred embodiment, the viral antigen to be separated is selected from the group consisting of a virus, a viral vector, a vaccine, a virus-like particle, or a combination thereof.

In another preferred embodiment, the viral antigen to be separated contains at least the following two substances:
1) a substance having a larger molecular weight in the separated sample, the molecular weight of the substance having a larger molecular weight is M1; and
2) a substance having a smaller molecular weight in the separated sample, and the molecular weight of the substance having a smaller molecular weight is M2;
M1/M2≥2.

In another preferred embodiment, the substance having a larger molecular weight is the target separation product.

In another preferred embodiment, the substance having a lower molecular weight is a process-related impurity.

In another preferred embodiment, M1/M2≥5, or M1/M2≥10.

In another preferred embodiment, the particle size of the separated viral antigen is 14-750 nm; preferably 16-300 nm; more preferably 18-200 nm; most preferably 20-120 nm.

In another preferred embodiment, the first buffer and the second buffer are the same or different, independently selected from the group consisting of a Tris buffer salt solution, a phosphate buffer salt solution, a NaCl salt solution, or a combination thereof.

In another preferred embodiment, the CIP solution is not particularly limited, such as NaOH aqueous solution, NaOH in ethanol/water mixed solution, NaOH in isopropanol/water mixed solution, preferably NaOH aqueous solution.

In another preferred embodiment, in step 4), the loading amount of the viral antigen to be separated is 1-2 column volumes.

In another preferred embodiment, in step 5), the flow rate of the rinsing is 10-1000 cm/h.

In another preferred embodiment, in step 5), the flow rate of the rinsing is in a range of 20-900 cm/h; preferably 50-800 cm/h; more preferably 100-700 cm/h; most preferably 150-500 cm/h.

In another preferred embodiment, in step 5), the operating pressure of the rinsing is ≤10 bar.

In another preferred embodiment, in step 5), the operating pressure of the rinsing is ≤5 bar; preferably ≤3 bar.

In another preferred embodiment, in the liquid chromatography method, the recovery rate of the viral antigen to be separated is ≥75%; preferably ≥80%; preferably ≥85%; more preferably ≥90%; most preferably ≥95%.

In another preferred embodiment, in the liquid chromatography method, the purity of the separated viral antigen is ≥80%; preferably ≥85%; more preferably ≥90%; most preferably ≥95%.

In the second aspect of the present invention, it provides a use of a chromatography medium in liquid chromatography for purifying and separating viral antigens;
the chromatography medium is a synthetic hydrophilic polymer, and has an essentially homogeneous porous structure from inside to outside of the medium and has a multilayer structure.

In another preferred embodiment, the chromatography medium is a synthetic hydrophilic polymer, has a porous structure, and has a core-shell two-layer structure;
the chromatography medium has amino groups on the pore surface in the core layer, and the shell layer has hydrophilic functional groups on the pore surface.

The chromatographic column packed with the chromatography medium of the present invention can have a variety of physical forms, and is consisting of a combination of the necessary components of different chromatographic columns.

Column packing method: constant flow/variable flow/constant pressure/multi-stage pressure adjustment/DAC.

According to the medium particle size, pore size and other properties and chromatography column specifications, different column packing methods are selected.

Constant flow method or constant pressure method can be used to pack columns if the medium particle size is smaller than 15 μm; constant flow method or variable flow method can be used to pack columns according to the intrinsic pressure resistance of chromatography medium and the targeted column dimensions if the medium particle size is larger than 15 μm.

Slurry preparation: water, salt water or water containing organic phase (20-80% v: v) can be selected. The appropriate slurry mobile phase and slurry volume should be selected according to the column specifications and chromatography medium performance.

In some embodiments, the chromatography column may be used in the range of pH 1-14; preferably 2-13; more preferably 4-10.

In some embodiments, the chromatography column may be used in a pressure range of <100 bar; preferably <50 bar; more preferably <10 bar; more preferably <5 bar; most preferably <3 bar.

Chromatography column rinsing conditions: the chromatographic column may adsorb some impurities that are difficult to clean after long-term use. These impurities will affect chromatographic performance, and these impurities need to be cleaned regularly. Different impurities have different cleaning methods. Generally, impurities are cleaned with 0.5 M HCl or 0.5-1.0 M NaOH. Impurities combined with strong hydrophobicity can be cleaned with 0.1-1% Tween and Triton X-100 or organic solvent additives.

Storage method of chromatographic column: The chromatographic column is stored in aqueous solution containing 20% ethanol at room temperature. The chromatography medium is stored in aqueous solution containing 20% ethanol at 2-8° C.

The chromatographic column or device can be combined with batch chromatography mode and continuous chromatography mode (such as counter flow chromatography). The chromatographic column can be used as a single column or multi-column form in continuous or non-continuous (conventional) chromatography. The column can be used for flow-through mode chromatography or binding-elution mode chromatography in analytical or industrial purification processes.

Another advantageous application of the medium is the separation of a mixture of biomolecules, the shell of which will exclude larger-sized cells, VLPs, vaccines, viral vectors or viruses, or liposomes, and prevent their interaction with functional groups on the pore surface in the core layer, such as ion exchange, affinity, hydrophobic or mixed ion-hydrophobic modes. Smaller size impurities, such as DNAs, RNAs, oligonucleotides, endotoxins, other small proteins and peptides, have adsorption effects on the functional groups on the pore surface in the core layer, and then they can be eluted with high salt or cleaning in place (CIP) reagents (such as 0.5-1.0 M NaOH aqueous based solution).

For example, Monomix Core 60 (part number: 290160990, further developed based on Resin 31 in the present invention) from SEPAX TECHNOLOGIES, INC. has been successfully applied to purify and separate viruses, virus vectors and virus-like particles (VLP). During the purification process, large-size viruses, viral vectors and virus-like particles (VLPs) are excluded from the shell layer of Monomix Core 60 and collected in flow-through mode, while most process-related impurities are temporarily absorbed by the mixed mode groups (amino groups) in the inner core layer and then removed from the medium through the CIP step. The chromatography medium aims to intermediately purify and finally polish biological macromolecular products. One-step chromatography purification can basically replace the two-step purification (size exclusion chromatography and anion exchange chromatography) in the traditional chromatography process. When the chromatography medium is used in the actual purification application of virus-like particles, the sample loading amount can reach at least 1 column volume (Example 73) in the flow through mode, far more than about 4% column volume of the conventional size exclusion chromatography medium in the adsorption-elution separation mode. The particle size and pore size of the chromatography medium are precisely controlled and can be adjusted according to application requirements. The surface chemical functional groups of the shell and core layer can be selected according to needs, and the density of the group can be adjusted and precisely controlled, and the high density and uniformity of the functional groups of the shell and core layer are ensured. The thickness of the shell and core layer can be adjusted and its uniformity is good.

The invention describes a liquid chromatography application for the purification and separation of viruses, viral vectors and vaccines. Viruses and viral vectors are widely used in gene therapy and vaccines.

The types of viruses include double-stranded DNA virus, single-stranded DNA virus, double-stranded RNA virus, positive-sense single-stranded RNA virus, anti-sense single-stranded RNA virus, single-stranded RNA retrovirus, and double-stranded DNA retrovirus.

Commonly used viruses and viral vectors include adenovirus, adeno-associated virus (AAV), lentivirus, human papillomavirus (HPV), herpes virus (HSV), bacteriophage and the corresponding viral vectors of the above viruses.

For the size of the virus, virus vector and virus-like particle, it is 14 nm-750 nm, preferably 16 nm-300 nm, further preferably 18 nm-200 nm, more preferably 20 nm-120 nm.

Vaccines can generally be divided into inactivated vaccines, live attenuated vaccines, replicating viral vector vaccines, non-replicating viral vector vaccines, virus-like particles, protein vaccines, DNA vaccines and RNA vaccines.

Virus-like particles (VLP) are virus empty protein shells, composed of virus shell proteins, and do not contain virus genetic material. Virus vector vaccine is preferably adenovirus vector vaccine. Adenovirus vectors can be human, animal or chimeric viruses, including recombinant human adenovirus vector type 5 (Ad5) and recombinant chimpanzee adenovirus vector type 26 (Ad26). It should be stated that inactivated vaccines, live attenuated vaccines, replicating viral vector vaccines, non-replicating viral vector vaccines, and virus-like particles are used in the development and commercialization of new corona vaccines for treating Covid-19.

Gene therapy integrates new therapeutic genomes into viral vectors, such as adenovirus, adeno-associated virus (AAV), lentivirus and other viruses. Therapeutic genes through viral vectors can be effectively delivered into cells to achieve genetic modification and diseased genes elimination. In the process of integrating the therapeutic genome into the viral vector, the novel mixed-mode chromatography medium in the present invention can be used to separate the free therapeutic gene and process-related impurities, and viral vector encapsulated with therapeutic gene. The viral vector encapsulated with therapeutic gene is size excluded by the shell layer because of its large size. Free therapeutic genes and process-related impurities can bind to the amino functional groups in the inner core layer to achieve separation.

The viruses commonly used in gene therapy are adenovirus, adeno-associated virus (AAV), lentivirus and viral vectors based on the above viruses.

It was reported that the recommended CIP conditions of Capto™ Core 700 was 1 M NaOH in 30% isopropanol aqueous solution, and CIP was required after each purification. This CIP process was inconvenient and affected production efficiency, because flammable and explosive organic solvents were used in the purification process, which were potential safety hazards. The optimized resin used in the present invention, Monomix Core 60 (part number: 290160990, further developed based on Resin 31 in the present invention) from SEPAX TECHNOLOGIES, INC., can be regenerated under mild CIP conditions: organic solvents (e.g. ethanol and isopropanol) may not be needed, and it may not be necessary to do CIP cleaning after each sample purification cycle.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A synthetic polymeric porous chromatography medium, wherein the chromatography medium has a hierarchical multiple layer structure, wherein the hierarchical multiple layer structure is made of synthetic polymer, has pores for size exclusion separation, and has an essentially homogeneous porous structure from inside to outside of the medium; and at least one inner layer and at least one outer layer in the hierarchical multiple layer structure have different binding functional groups (or liquid chromatography (LC) functional groups), or have same binding functional group with different density so that the chromatographic property of said at least one inner layer is different from that of said at least one outer layer.

2. The synthetic polymeric porous chromatography medium of claim 1, wherein the chromatography medium has core-shell(s) structure.

3. The synthetic polymeric porous chromatography medium of claim 1, wherein the hierarchical multiple layer structure has 2, 3, or 4 layers and the porous structure between different layers are essentially the same as supported by the average pore sizes between different layers.

4. The synthetic polymeric porous chromatography medium of claim 1, wherein the binding function group is selected from the group consisting of hydrophobic groups, hydrophilic groups, ionic or ionizable groups, affinity groups, mixed-mode groups and combinations of two or more thereof.

5. The synthetic polymeric porous chromatography medium of claim 1, wherein the chromatography medium has one or more of the following features:
(a) specific pore volume in a range of 0.05-3.0 mL/g;
(b) specific surface area in a range of 40-1200 m²/g;
(c) average pore size in a range of 30-5000 Å;
(d) volume average particle diameter (D₅₀) in a range of 1-1000 μm;
(e) particle size distribution (D₉₀/D₁₀) in a range of 1.0-2.2.

6. The synthetic polymeric porous chromatography medium of claim 1, wherein the chromatography medium is made from a mother medium.

7. The synthetic polymeric porous chromatography medium of claim 6, wherein the mother medium is copolymerized from a monomer mixture which comprises:
(M1) at least a first monomer which is a crosslinking monomer;
(M2) at least a second monomer which comprises a monomer with a convertible functional group for hierarchical structure construction, and
(M3) an optional third monomer which has a special functional group for tuning chromatographic properties.

8. The synthetic polymeric porous chromatography medium of claim 6, wherein the mother medium has one or more of the following features:
(a) specific pore volume in a range of 0.05-3.0 mL/g;
(b) specific surface area in a range of 40-1200 m²/g;
(c) average pore size in a range of 30-5000 Å;
(d) volume average particle diameter (D₅₀) in a range of 1-1000 μm;
(e) particle size distribution (D₉₀/D₁₀) in a range of 1.0-2.2;
(f) alkene content of the mother medium in a range of 0.5-6.0 mmol/g;
(g) the average pore size is essentially homogeneous from inside to outside of the porous mother medium.

9. The synthetic polymeric porous chromatography medium of claim 1, wherein the shape and/or form of the chromatography medium is a substantially flat particulate or monolithic rod or disk.

10. The synthetic polymeric porous chromatography medium of claim 7, wherein the mother medium has one or more of the following features:
(F1) the first monomer or crosslinking monomer accounts for 1-99% wt of all monomers used in copolymerization process;
said crosslinking monomer is selected from the group consisting of divinylbenzene (DVB), ethylene glycol dimethacrylate, pentaerythritol dimethacrylate, pentaerythritol triacrylate, pentaerythritol tetraacrylate, sorbitol dimethacrylate, poly(ethylene glycol) diacrylate, poly(propylene glycol) diacrylate, trimethylolpropane triacrylate, bis2-(methacryloyloxy)ethyl phosphate, N,N′-methylenebisacrylamide, 3-(acryloyloxy)-2-hydroxypropyl methacrylate, glycerol 1,3-diglycerolate diacrylate, 1,5-hexadiene, allyl ether, diallyl diglycol carbonate, di(ethylene glycol) bis(allyl carbonate), ethylene glycol bis(allyl carbonate), triethylene glycol bis(allyl carbonate), tetraethylene glycol bis(allyl carbonate), glycerol tris (allyl carbonate), ethylene glycol bis(methallyl carbonate), diallyl phthalate, triallyl isocyanurate, diallyl isophthalate, diallyl terephthalate, diallyl itaconate, diallyl 2,6-naphthalene dicarboxylate, diallyl chlorendate, triallyl trimellitate, triallyl citrate, 2,4,6-triallyloxy-1,3,5-triazine, 1,3,5-triacryloylhexahydro-1,3,5-triazine, glyoxal bis(diallyl acetal), N,N-diallyldimethyl ammonium salts, ethylene glycol diallyl ether and combinations of two or more thereof;
(F2) the second monomer accounts for 1-99% wt of all monomers used in copolymerization process;
the second monomer is selected from the group consisting of allyl acrylate, allyl methacrylate, vinyl acrylate, diallyl maleate, (meth)acrylate, acrylamide, ethylene terephthalate, ethylene, propylene, styrene, vinyl acetate, vinyl acrylate, vinyl chloride, vinyl pyrrolidone, DVB, 1,3,5-trivinylbenzene, and combinations of two or more thereof;
(F3) the third monomer accounts for 1-99% wt of all monomers used in copolymerization process;
said third monomer is selected from the group consisting of glycidyl methacrylate, 2-hydroxyethyl methacrylate, 2-carboxyethyl acrylate, 2-hydroxyethyl acrylate, 2-hydroxyethyl methacrylate, methyl methacrylate, methacrylic acid, hydroxypropyl methacrylate, 2-(methacryloyloxy)ethyl acetoacetate, mono-2-(methacryloyloxy)ethyl maleate, benzyl acrylate, butyl acrylate, styrene, DVB, N-Vinylpyrrolidone and combinations of two or more thereof.

11. The synthetic polymeric porous chromatography medium of claim 2, wherein the chromatography medium has one or more of the following features:
(T1) the chromatography medium with core-shell(s) structure has at least two layers, where the core refers to the most inner layer, while the shell(s) refer(s) to the outer layer(s) from the core;
(T2) the chromatography medium with core-shell structure is composed of a hydrophilic shell and cationic ligand(s) activated core with or without a linker;
(T3) the chromatography medium with core-shell structure is composed of a hydrophilic shell and anionic ligand(s) activated core with or without a linker;
(T4) the chromatography medium with core-shell structure is composed of a hydrophilic shell and hydrophobic ligand(s) activated core with or without a linker;
(T5) the chromatography medium with core-shell structure is composed of a hydrophilic shell and affinity ligand(s) activated core with or without a linker;
(T6) the chromatography medium with core-shell structure is composed of a hydrophilic shell and mixed-mode ligand(s) activated core with or without a linker;
(T7) the chromatography medium has a cationic shell, which is modified with any suitable reagent leading to positively charged ligands, and a hydrophobic ligand(s) activated core, which can carry any hydrophobic ligands;
(T8) the chromatography medium has an anionic shell, which is modified with any suitable reagent leading to negatively charged ligands, and a hydrophobic ligand(s) activated core, which can carry any hydrophobic ligands;
(T9) the chromatography medium has an ionic or ionizable shell, which is modified with any suitable reagent leading to said ionic or ionizable ligands, and a hydrophilic core;
(T10) the chromatography medium is modified with the same ligand(s) in both the core layer and the shell layer, but with a different functional group(s) density;
(T11) the hydrophilicity in each shell of the chromatography medium can be tuned and enhanced through chemical modification with 2-hydroxyethanethiol, 3-sulfanylpropane-1,2-diol, Dextran, any linear or branched multifunctional epoxide, or any other agents with hydrophilic functional groups;
(T12) the chromatography medium can be physically converted/transformed into LC columns or other confined devices for molecular separations and purifications;
(T13) the chromatography medium with core-shell two-layer structure and designed pore size is used for analytical and preparative separations;
(T14) the chromatography medium combines anionic exchange adsorptive and size exclusion mechanisms, where a large substance is analyzed or collected in flow-through mode, while a small substance is temporarily trapped/bound in the core, and then eluted for analysis or collection;
(T15) the chromatography medium combines cationic exchange adsorptive and size exclusion mechanisms, where a large substance, is analyzed or collected in flow-through mode, while a small substance is temporarily trapped/bound in the core, and then eluted for analysis or collection;
(T16) the chromatography medium combines hydrophobic adsorptive and size exclusion mechanisms, where a large substance, is analyzed or collected in flow-through mode, while a small substance is temporarily trapped/bound in the core, and then eluted for analysis or collection;
(T17) the chromatography medium combines affinity adsorptive and size exclusion mechanisms, where a large substance, is analyzed or collected in flow-through mode, while a small substance is temporarily trapped/bound in the core, and then eluted for analysis or collection;
(T18) the chromatography medium combines mixed-mode adsorptive and size exclusion mechanisms, where a large substance, is analyzed or collected in flow-through mode, while a small substance is temporarily trapped/bound in the core, and then eluted for analysis or collection;
(T19) the chromatography medium is applied to separate biomolecules from the surfactants used in stabilizing biotherapeutic formulation;
(T20) the chromatography medium is applied to separate mixtures of large or super biomolecule assemblies natural or artificially-made from small molecules or assemblies via the interactions at inner core different from that at outer layer.

12. The synthetic polymeric porous chromatography medium of claim 1, wherein the chromatography medium has an affinity ligand.

13. The synthetic polymeric porous chromatography medium of claim 12, wherein the chromatography medium is selected from the group consisting of:
(A1) a chromatography medium bearing an affinity ligand Protein A attached in its inner core;
(A2) a chromatography medium bearing an affinity ligand Protein L attached in the inner core;
(A3) a chromatography medium bearing an affinity ligand Protein G attached in the inner core;
(A4) a chromatography medium bearing an affinity ligand oligonucleotide.

14. The synthetic polymeric porous chromatography medium of claim 2, wherein the chromatography medium has a ratio of thickness of the shell layer to total thickness of the shell layer and the core layer of 0.5%-30%.

15. The synthetic polymeric porous chromatography medium of claim 2, wherein the chromatography medium has a thickness of the shell layer of 0.5-10 μm.

16. The synthetic polymeric porous chromatography medium of claim 2, wherein when the functional group of the core layer is the same to that of the shell layer, the functional group density of the core layer is D1, the functional group density of the shell layer is D2, and the chromatography medium has one of the following features:
1) D1/D2 is larger than 1.05;
2) D2/D1 is larger than 1.05.

17. A synthetic polymeric porous mother medium, wherein the mother medium is copolymerized from a monomer mixture which comprises:
(M1) at least a first monomer which is a crosslinking monomer;
(M2) at least a second monomer which comprises a monomer with a convertible functional group for hierarchical structure construction, and
(M3) an optional third monomer which has a special functional group for tuning chromatographic property.

18. The synthetic polymeric porous mother medium of claim 17, wherein the mother medium has one or more of the following features:
(a) specific pore volume in a range of 0.05-3.0 mL/g;
(b) specific surface area in a range of 40-1200 m²/g;
(c) average pore size in a range of 30-5000 Å;
(d) volume average particle diameter (D₅₀) in a range of 1-1000 μm;
(e) particle size distribution (D₉₀/D₁₀) in a range of 1.0-2.2;
(f) alkene content of the mother medium in a range of 0.5-6.0 mmol/g;
(g) the shape and/or form of the mother medium is a substantially flat particulate or monolithic rod or disk;
(h) the mother medium has the convertible functional group and/or the special functional group for tuning chromatographic property at the outside surface and inner portion thereof;
(i) the average pore size is essentially homogeneous from inside to outside of the porous mother medium.

19. A solid support, wherein the solid support comprises:
1) the synthetic polymeric porous chromatography medium of claim 1; and
2) a detectable label conjugated on the chromatography medium of claim 1.

20. The solid support of claim 19, wherein the detectable label is selected from the group consisting of: protein, enzyme, catalyst, dye, fluorescent group, luminescent group, and combinations of two or more thereof.

21. A method for preparing the synthetic polymeric porous chromatography medium of claim 1, which comprises:
(a) providing a synthetic polymeric porous mother medium, wherein the mother medium is copolymerized from a monomer mixture which comprises:
(M1) at least a first monomer which is a crosslinking monomer;
(M2) at least a second monomer which comprises a monomer with a convertible functional group for hierarchical structure construction, and
(M3) an optional third monomer which has a special functional group for tuning chromatographic property;
(b) modifying the convertible functional group and/or the special functional group, thereby obtaining the synthetic polymeric porous chromatography medium of claim 1.

22. The method of claim 21, which comprises the following steps:
(Z1) providing the synthetic polymeric porous mother medium;
(Z2) adding a modification reagent to modify the convertible functional group of the mother medium, thereby obtaining an intermediate medium with chemically distinct two-layer structure, wherein the thickness of the shell layer is controlled by adjusting the adding amount of the modification reagent;
(Z3) modifying the resulting group(s) obtained in step (Z2), to construct the shell layer of said medium with suitable binding functional group(s) depending on the separation needs;
(Z4) adding modification reagent(s) to modify the convertible functional group(s) in the core layer of said intermediate medium;
(Z5) modifying the resulting group(s) obtained in step (Z4) with suitable ligand(s) to construct the core of corresponding intermediate medium with suitable binding functional group(s), thereby obtaining a chromatography medium with different binding functional groups inside and outside the chromatography medium or with same binding functional groups having a different density inside and outside the chromatography medium.

23. The method of claim 21, which comprises the following steps:
(Y1) providing the synthetic polymeric porous mother medium;
(Y2) filling the inside of the mother medium with an inert filling;
(Y3) adding modification reagent(s) to modify the convertible functional group outside the mother medium to obtain an intermediate medium with chemically distinct two-layer structure;
(Y4) modifying the resulting group obtained in step (Y3), to construct the shell layer of said medium with suitable binding functional group(s) depending on the separation needs;
(Y5) removing the inert filling from inside of the mother medium;
(Y6) adding modification reagent(s) to modify the convertible functional group inside the mother medium;
(Y7) modifying the resulting group obtained in step (Y6) to obtain a second binding functional group inside the mother medium, thereby obtaining a chromatography medium with different binding functional groups inside and outside the chromatography medium or with same binding functional groups having a different density inside and outside the chromatography medium.

24. The method of claim 23, wherein the inert fillings are in liquid, gel/semi-solid or solid, regardless of their molecular weight and sizes.

25. A method for preparing the mother medium of claim 17, which comprises the steps of:
(S1) providing a monomer mixture which comprises:
(M1) at least a first monomer which is a crosslinking monomer;
(M2) at least a second monomer which comprises a monomer with a convertible functional group for hierarchical structure construction, and
(M3) an optional third monomer which has a special functional group for tuning chromatographic property; and
(S2) conducting a copolymerization process to obtain the mother medium of claim 17.

26. The method of claim 25, wherein a porogen is used during the copolymerization process, and the method has one or more of the following features:
B1) the porogen is selected from the group consisting of hexanes, pentanes, octanes, pentanols, hexanols, heptanols, octanols, methyl isobutyl carbinol, cyclohexanol, toluene and xylenes, ethyl acetate, diethyl phthalate, and dibutyl phthalate, poly(propylene glycol), and poly(ethylene glycol);
B2) the weight ratio of total amount of porogens to total amount of monomers is 10%-400%;
B3) the weight ratio of one single porogen to total weight of porogen is 0.1%-99.9%.

27. The method of claim 25, wherein a swellable polymer/oligomer seed is used during the copolymerization process, and the method has one or more of the following features:
C1) the swellable polymer/oligomer seed is selected from the group consisting of (meth)acrylic, styrenic, oligostyrene, oligoacrylates, oligo-BMA, oligo-BA, vinyl acetate, and combinations thereof;
C2) the seed has a MW less than 70,000 g/mol for primary seed and 10,000 g/mol for later stage seed;
C3) the swelling ratio in each seeding process is 2-300, preferably 5-200.

28. (canceled)

29. (canceled)

30. A liquid chromatography method for purifying and separating biologics, characterized in that the method comprises the following steps:
1) providing the chromatography medium of claim 1, biologics to be separated, first buffer, second buffer, and cleaning in place (CIP) solution;
wherein the chromatography medium is a synthetic polymer, has a porous structure, and has a 2-5 layered structures;
2) packing the liquid chromatography column with the chromatography medium, and the liquid chromatography column using the above method is obtained;
3) rinsing the liquid chromatography column with the first buffer;
4) loading the biologics to be separated into the liquid chromatography column obtained in step 3);
5) rinsing the liquid chromatography column obtained in step 4) with the second buffer, collecting the separated product to obtain the separated biologics;
6) rinsing the liquid chromatography column obtained in step 5) with the CIP solution, collecting the separated product, and removing the process-related impurities in the biologics.

31.-33. (canceled)

34. The liquid chromatography method of claim 30, wherein the liquid chromatography column has one or more characteristics selected from the group consisting of:
1) the ion exchange equivalent of the liquid chromatography column chromatography medium in the core layer is 100-500 μmol/mL;
2) the linear flow rate of the liquid chromatography column is 10 cm/h-1000 cm/h;
3) the operating pressure of the liquid chromatography column is ≤100 bar.

35. (canceled)

36. The liquid chromatography method of claim 30, wherein in step 4), the loading amount of the biologics to be separated is in a range of 0.001-20 column volumes.

37. The liquid chromatography method of claim 30, wherein in step 3-6), the flow rate of the liquid media is 10 cm/h-1000 cm/h.

38. The liquid chromatography method of claim 30, wherein in step 3-6), the operation pressure of the liquid media is ≤10 bar.

39. The liquid chromatography method of claim 30, wherein the biologics to be separated is selected from the group consisting of lipids, proteins, antibodies, plasmids, RNAs, DNAs, VLPs, antigens, vaccines, viral vectors, viruses, bacteria.
